(19) United States
(12) Patent Application Publication
(10) Pub. No.: US 2024/0423924 A1
(43) Pub. Date: Dec. 26, 2024

(54) PREVOTELLA EXTRACELLULAR VESICLE PREPARATIONS

(71) Applicant: Evelo Biosciences, Inc., Cambridge, MA (US)

(72) Inventors: Shannon ARGUETA, Cambridge, MA (US); Adam Cartwright, Cambridge, MA (US); Derek Corman, Billerica, MA (US); Tanmoy Ganguly, Cambridge, MA (US); Andrea Itano, Arlington, MA (US); Collin McKenna, Cambridge, MA (US); Divya Raghunathan, Cambridge, MA (US); Bill Wang, Billerica, MA (US)

(21) Appl. No.: 18/274,088

(22) PCT Filed: Jan. 25, 2022
(86) PCT No.: PCT/US2022/013716

§ 371 Date: Jul. 25, 2023

Related U.S. Application Data
(60) Provisional application No. 63/289,348, filed on Dec. 14, 2021.
(60) Provisional application No. 63/196,984, filed on Jun. 4, 2021.
(60) Provisional application No. 63/175,855, filed on Apr. 16, 2021.
(60) Provisional application No. 63/141,693, filed on Jan. 26, 2021.

(51) Int. Cl. A61K 9/50; A61K 9/00; A61K 9/48; A61K 35/74; A61P 37/06

(52) U.S. Cl. CPC A61K 9/5068; A61K 9/0053; A61K 9/4858; A61K 35/74; A61P 37/06

(57) ABSTRACT

Provided herein are Prevotella histicola extracellular vesicles (EVs), solutions, and dried forms (and therapeutic compositions thereof) of Prevotella histicola extracellular vesicles (EVs) that can be useful as therapeutic agents, and methods of use thereof.

### CROSS-REFERENCE TO RELATED APPLICATIONS

This application claims the benefit of the following U.S. Provisional application Nos.: 63/141,693, filed Jan. 26, 2021; 63/175,855, filed Apr. 16, 2021; 63/196,984, filed Jun. 4, 2021; and 63/289,348, filed on Dec. 14, 2021, the entire contents of each are incorporated herein by reference.

### SUMMARY

Therapeutic compositions comprising extracellular vesicles (EVs), such as EVs obtained from Prevotella histicola bacteria, have therapeutic effects and are useful for the treatment and/or prevention of disease and/or health disorders. As described herein, EVs from Prevotella histicola bacteria can be prepared as a biomass (e.g., isolated EVs can be resuspended in a buffer such as PBS). As described herein, EVs from Prevotella histicola bacteria can be prepared as solutions, dried forms and/or therapeutic compositions.

In some embodiments, dried forms having a moisture content below about 6% are better suited for downstream processing, In some embodiments, dried forms having a moisture content below about 6% have improved stability. In some embodiments, the solutions comprising the EVs from Prevotella histicola bacteria also comprise an excipient that contains a bulking agent, and optionally comprises one or more additional ingredients, such as a lyoprotectant. In some embodiments, the solutions comprising the EVs from Prevotella histicola bacteria also comprise an excipient that contains a lyoprotectant, and optionally comprises one or more additional ingredients, such as a bulking agent. In some embodiments, the dried forms comprising the EVs from Prevotella histicola bacteria also comprise an excipient that contains a bulking agent, and that optionally comprises one or more additional ingredients, such as a lyoprotectant. In some embodiments, the dried forms comprising the EVs from Prevotella histicola bacteria also comprise an excipient that contains a lyoprotectant, and optionally comprise one or more additional ingredients, such as a bulking agent.

In certain aspects, provided herein is a method of treating an immune disorder in a subject (e.g., a human subject) comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles. In some embodiments, the immune disorder comprises an autoimmune disease, an inflammatory disease, or an allergy. In some embodiments, the immune disorder comprises an inflammatory disease.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for treating an immune disorder in a subject (e.g., a human subject).

In certain aspects, provided herein is a dose (e.g., a therapeutically effective dose) extracellular vesicles (EVs) from a Prevotella histicola strain and/or a composition (e.g., a solution, dried form and/or therapeutic composition) comprising the extracellular vesicles for use in treating an immune disorder in a subject (e.g., a human subject).

In certain aspects, provided herein is a method of treating an inflammatory disease in a subject (e.g., a human subject) comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles. In some embodiments, the inflammatory disease comprises a Th1 mediated inflammatory disease. In some embodiments, the inflammatory disease comprises a Th2 mediated inflammatory disease (such as asthma or atopic dermatitis). In some embodiments, the inflammatory disease comprises a Th17 mediated inflammatory disease (such as psoriasis).

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for treating an inflammatory disease in a subject (e.g., a human subject).

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in treating an inflammatory disease in a subject (e.g., a human subject).

In certain aspects, provided herein is a method of activating TLR2 in a subject (e.g., a human subject) comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for the activation of TLR2 in a subject (e.g., a human subject).

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in the activation of TLR2 in a subject (e.g., a human subject).

In certain aspects, provided herein is a method of stimulating interleukin-10 receptor (IL-10R) in a subject (e.g., a human subject) comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for stimulating interleukin-10 receptor (IL-10R) in a subject (e.g., a human subject).

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in stimulating interleukin-10 receptor (IL-10R) in a subject (e.g., a human subject).

In certain aspects, provided herein is a method of activating an anti-inflammatory cytokine response in a subject (e.g., a human subject) comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles. In some embodiments, the anti-inflammatory cytokine response comprises interleukin-10 (IL-10) production. In some embodiments, the anti-inflammatory cytokine response comprises IL-27 production. In some embodiments, the anti-inflammatory cytokine response comprises IL-10 and IL-27 production.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for activating an anti-inflammatory cytokine response in a subject (e.g., a human subject).

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in activating an anti-inflammatory cytokine response in a subject (e.g., a human subject).

In certain aspects, provided herein is a method of increasing anti-inflammatory cytokine secretion in a subject (e.g., a human subject) (e.g., in PBMCs thereof) comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles. In some embodiments, the anti-inflammatory cytokine is IL-10. In some embodiments, the anti-inflammatory cytokine is IL-27. In some embodiments, the anti-inflammatory cytokines are IL-10 and IL-27.

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for increasing anti-inflammatory cytokine secretion in a subject (e.g., a human subject).

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in increasing anti-inflammatory cytokine secretion in a subject (e.g., a human subject).

In certain aspects, provided herein is a method of activating TLR1/2 and/or TLR2/6 heterodimers in a subject (e.g., a human subject) comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in activating TLR1/2 and/or TLR2/6 heterodimers in a subject (e.g., a human subject).

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for activating TLR1/2 and/or TLR2/6 heterodimers in a subject (e.g., a human subject).

In some embodiments, the Prevotella histicola strain activates TLR1/2 and/or TLR2/6 heterodimers, e.g., in an in vitro assay, e.g., as described herein.

In certain aspects, provided herein is a method of instructing T cells to be less inflammatory in a subject (e.g., a human subject) comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in instructing T cells to be less inflammatory in a subject (e.g., a human subject).

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for instructing T cells to be less inflammatory in a subject (e.g., a human subject).

In some embodiments, the T cells are instructed in mesenteric lymph nodes.

In some embodiments, the extracellular vesicles (EVs) from a Prevotella histicola strain and/or a composition (e.g., a solution, dried form and/or therapeutic composition) comprising the extracellular vesicles are orally administered (e.g., and travels to the small intestine), dendritic cells interact with the extracellular vesicles (EVs) from a Prevotella histicola strain and/or a composition (e.g., a solution, dried form and/or therapeutic composition) comprising the extracellular vesicles in the small intestine, the dendritic cells travel to the mesenteric lymph nodes, and T cells trafficking through the mesenteric lymph node encounter the dendritic cells.

In certain aspects, provided herein is a method of affecting T cells that traffic to mesenteric lymph nodes in a subject (e.g., a human subject) comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in affecting T cells that traffic to mesenteric lymph nodes in a subject (e.g., a human subject).

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for affecting T cells that traffic to mesenteric lymph nodes in a subject (e.g., a human subject).

In certain aspects, provided herein is a method of generating inflammation-resolving CD4+ T cells in a subject (e.g., a human subject) comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles. In some embodiments, as demonstrated in the examples provided herein, the extracellular vesicles generate a population of CD4+ T cells that can resolve inflammation.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in generating inflammation-resolving CD4+ T cells in a subject (e.g., a human subject).

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for generating inflammation-resolving CD4+ T cells in a subject (e.g., a human subject).

In certain aspects, provided herein is a method of resolving inflammation in a subject (e.g., a human subject) comprising administering (e.g., orally administering) to the subject a dose (e.g., a therapeutically effective dose) of a dose (e.g., a therapeutically effective dose) of extracellular vesicles (EVs) from a Prevotella histicola strain and/or a composition (e.g., a solution, dried form and/or therapeutic composition) comprising the extracellular vesicles. In some embodiments, as demonstrated in the examples provided herein, the extracellular vesicles dosed in the absence of inflammation do not inhibit the immune response, but instead, resolve an ongoing inflammatory response. In some embodiments, as described in the examples herein, an antigen-independent mechanism that can reduce antigen-specific inflammation.

In certain aspects, provided herein is a dose (e.g., a therapeutically effective dose) of extracellular vesicles (EVs) from a Prevotella histicola strain and/or a composition (e.g., a solution, dried form and/or therapeutic composition) comprising the extracellular vesicles for use in resolving inflammation in a subject (e.g., a human subject).

In certain aspects, provided herein is use of a dose (e.g., a therapeutically effective dose) of extracellular vesicles (EVs) from a Prevotella histicola strain and/or a composition (e.g., a solution, dried form and/or therapeutic composition) comprising the extracellular vesicles for the preparation of a medicament for resolving inflammation in a subject (e.g., a human subject).

In some embodiments, the subject (e.g., a human subject) has Th1 mediated inflammation.

In some embodiments, the subject (e.g., a human subject) has Th2 mediated inflammation.

In some embodiments, the subject (e.g., a human subject) has Th17 mediated inflammation.

In some embodiments of the methods provided herein, the dose (e.g., a therapeutically effective dose) of extracellular vesicles (EVs) from a Prevotella histicola strain and/or a composition (e.g., a solution, dried form and/or therapeutic composition) comprising the extracellular vesicles is administered in combination with an anti-TNF alpha antibody. In some embodiments of the methods provided herein, the dose (e.g., a therapeutically effective dose) of extracellular vesicles (EVs) from a Prevotella histicola strain and/or a composition (e.g., a solution, dried form and/or therapeutic composition) comprising the extracellular vesicles is administered in combination with an TNF alpha antagonist (e.g., TNF alpha antagonist or TNF alpha receptor antagonist), e.g., ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; Mab Thera®), ABATACEPT (Orencia®), TOCILIZUMAB (RoActemra/Actemra®),

In some embodiments, the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329). In some embodiments, the Prevotella histicola strain is the Prevotella histicola Strain B (NRRL accession number B 50329).

In some embodiments, the subject (e.g., a human subject) has an immune disorder. In some embodiments, the immune disorder is arthrosclerosis, arthritis, phlebitis, vasculitis, and lymphangitis. cholangitis, cholecystitis, enteritis, enterocolitis, gastritis, gastroenteritis, inflammatory bowel disease, ileitis, proctitis, Crohn's disease, ulcerative colitis, irritable bowel syndrome, microscopic colitis, lymphocytic-plasmocytic enteritis, coeliac disease, collagenous colitis, lymphocytic colitis, eosinophilic enterocolitis, indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, primary sclerosing cholangitis, cervicitis, chorioamnionitis, endometritis, epididymitis, omphalitis, oophoritis, orchitis, salpingitis, tubo-ovarian abscess, urethritis, vaginitis, vulvitis, vulvodynia, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, diabetes mellitus type 1, giant cell arteritis, good pasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, Ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, contact hypersensitivity, contact dermatitis (including that due to poison ivy), urticaria, skin allergies, respiratory allergies (hay fever, allergic rhinitis, house dust mite allergy), appendicitis, dermatitis, dermatomyositis, endocarditis, fibrositis, gingivitis, glossitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, stomatitis, transplant rejection, acute pancreatitis, chronic pancreatitis, acute respiratory distress syndrome, Sexary's syndrome, congenital adrenal hyperplasia, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenia purpura in adults, secondary thrombocytopenia in adults, acquired (autoimmune) haemolytic anemia, leukemia and lymphomas in adults, acute leukemia of childhood, regional enteritis, autoimmune vasculitis, chronic obstructive pulmonary disease, or sepsis.

In some embodiments, the subject (e.g., a human subject) has psoriasis.

In some embodiments, the subject (e.g., a human subject) has atopic dermatitis.

In some embodiments, the subject (e.g., a human subject) has a Th1 mediated inflammatory disease.

In some embodiments, the subject (e.g., a human subject) has a Th2 mediated inflammatory disease.

In some embodiments, the subject (e.g., a human subject) has a Th17 mediated inflammatory disease.

In some embodiments, the EVs are administered orally.

In certain aspects, the extracellular vesicles (EVs) from a Prevotella histicola strain and/or a composition (e.g., a solution, dried form and/or therapeutic composition) comprising the extracellular vesicles reduce inflammation in a DTH model of inflammation.

In certain aspects, the extracellular vesicles (EVs) from a Prevotella histicola strain and/or a composition (e.g., a solution, dried form and/or therapeutic composition) comprising the extracellular vesicles stimulate TLR2 in an in vitro cultured HEK293 reporter cell line assay.

In certain aspects, the extracellular vesicles (EVs) from a Prevotella histicola strain and/or a composition (e.g., a solution, dried form and/or therapeutic composition) comprising the extracellular vesicles stimulate IL-10 secretion from in vitro cultured U937 cells (e.g., that have been differentiated with PMA treatment).

In certain aspects, the extracellular vesicles (EVs) from a Prevotella histicola strain and/or a composition (e.g., a solution, dried form and/or therapeutic composition) comprising the extracellular vesicles stimulate IL-10 secretion from in vitro cultured human PBMCs.

In certain aspects, the extracellular vesicles (EVs) from a Prevotella histicola strain and/or a composition (e.g., a solution, dried form and/or therapeutic composition) comprising the extracellular vesicles stimulate IL-27 secretion from in vitro cultured human PBMCs.

In certain aspects, the extracellular vesicles (EVs) from a Prevotella histicola strain and/or a composition (e.g., a solution, dried form and/or therapeutic composition) comprising the extracellular vesicles induce IL-10, IL-27, IL-6, IP-10, and/or TNFa secretion from in vitro cultured human PBMCs.

In certain aspects, the extracellular vesicles (EVs) from a Prevotella histicola strain and/or a composition (e.g., a solution, dried form and/or therapeutic composition) comprising the extracellular vesicles induce IL-10, IL-27, IL-6, IP-10, and/or TNFa secretion from in vitro cultured human macrophages.

In certain aspects, the extracellular vesicles (EVs) from a Prevotella histicola strain and/or a composition (e.g., a solution, dried form and/or therapeutic composition) comprising the extracellular vesicles induce IL-10, IL-27, IL-6, IP-10, and/or TNFa secretion from in vitro cultured human dendritic cells. In certain aspects, the extracellular vesicles (EVs) from a Prevotella histicola strain and/or a composition (e.g., a solution, dried form and/or therapeutic composition) comprising the extracellular vesicles induce IL-10, IL-6, and/or TNFa secretion from in vitro cultured human dendritic cells.

In certain aspects, the extracellular vesicles (EVs) from a Prevotella histicola strain and/or a composition (e.g., a solution, dried form and/or therapeutic composition) comprising the extracellular vesicles induce IL-10, IL-27, IL-6, IP-10, and TNFa secretion from in vitro cultured U937 cells.

In some embodiments, the dose is in the form of one or more capsules, optionally comprising an enteric-coating (e.g., enteric-coated capsules). In some embodiments, the dose is in the form of one or more tablets, optionally comprising an enteric-coating (e.g., enteric-coated tablets). In some embodiments, the dose is in the form of one or more mini-tablets. In some embodiments, the mini-tablets are enteric-coated mini-tablets. In some embodiments, the dose is in the form of a non-enteric coated capsule comprising one or more enteric-coated mini-tablets.

As disclosed herein, Prevotella histicola extracellular vesicles (EVs) have therapeutic effects and are useful for the treatment and/or prevention of disease and/or health disorders. Therapeutic compositions of biomass, solutions and dried forms containing Prevotella histicola EVs can be prepared.

Bulking agents and/or lyoprotectants are used when preparing extracellular vesicles (EVs) for drying, such as freeze drying and spray drying. In some embodiments, bulking agents, including but not limited to sucrose, mannitol, polyethylene glycol (PEG, such as PEG 6000), cyclodextrin, maltodextrin, and dextran (such as dextran 40k), make dried forms (such as powders and/or lyophilates) easier to handle after drying. In some embodiments, bulking agents improve the properties of a dried form. In some embodiments, lyoprotectants, including but not limited to trehalose, sucrose, and lactose protect the EVs during drying, such as freeze-drying or spray drying. In some embodiments, the excipient functions to decrease drying cycle time. In some embodiments, the excipient functions to maintain therapeutic efficacy of the EVs.

In some aspects, the disclosure provides a dried form comprising extracellular vesicles (EVs) from Prevotella histicola bacteria, wherein the dried form has a moisture content (e.g., as determined by the Karl Fischer method) of below about 6%.

In some embodiments, the dried form provided herein has a moisture content (e.g., as determined by the Karl Fischer method) of below about 5%.

In some embodiments, the dried form provided herein has a moisture content (e.g., as determined by the Karl Fischer method) of below about 4%.

In some embodiments, the dried form provided herein has a moisture content (e.g., as determined by the Karl Fischer method) of between about 1% to about 4%.

In some embodiments, the dried form provided herein has a moisture content (e.g., as determined by the Karl Fischer method) of between about 2% to about 4%.

In some embodiments, the dried form provided herein has a moisture content (e.g., as determined by the Karl Fischer method) of between about 2% to about 3%.

In some aspects, the disclosure provides a lyophilate comprising extracellular vesicles (EVs) from Prevotella histicola bacteria, wherein the lyophilate has a moisture content (e.g., as determined by the Karl Fischer method) of below about 6%.

In some embodiments, the lyophilate has a moisture content (e.g., as determined by the Karl Fischer method) of below about 5%.

In some embodiments, the lyophilate has a moisture content (e.g., as determined by the Karl Fischer method) of below about 4%.

In some embodiments, the lyophilate has a moisture content (e.g., as determined by the Karl Fischer method) of between about 1% to about 4%.

In some embodiments, the lyophilate has a moisture content (e.g., as determined by the Karl Fischer method) of between about 2% to about 4%.

In some embodiments, the lyophilate has a moisture content (e.g., as determined by the Karl Fischer method) of between about 2% to about 3%.

In some aspects, the disclosure provides a powder comprising extracellular vesicles (EVs) from Prevotella histicola bacteria, wherein the powder has a moisture content (e.g., as determined by the Karl Fischer method) of below about 6%.

In some embodiments, the powder has a moisture content (e.g., as determined by the Karl Fischer method) of below about 5%.

In some embodiments, the powder has a moisture content (e.g., as determined by the Karl Fischer method) of below about 4%.

In some embodiments, the powder has a moisture content (e.g., as determined by the Karl Fischer method) of between about 1% to about 4%.

In some embodiments, the powder has a moisture content (e.g., as determined by the Karl Fischer method) of between about 2% to about 4%.

In some embodiments, the powder has a moisture content (e.g., as determined by the Karl Fischer method) of between about 2% to about 3%.

In some aspects, the disclosure provides a dried form comprising extracellular vesicles (EVs) from Prevotella histicola bacteria and an excipient, wherein the excipient comprises about 95% to about 99% of the total mass of the dried form.

In some aspects, the disclosure provides a dried form comprising extracellular vesicles (EVs) from Prevotella histicola bacteria and an excipient, wherein the EVs can make up about 2% to about 6% of the total mass of the dried form.

In some embodiments of the dried form provided herein, the dried form comprises a powder. In some embodiments, the powder comprises a lyophilized powder. In some embodiments the powder comprises a spray-dried powder.

In some embodiments of the dried form provided herein, the dried form comprises a lyophilate. In some embodiments, the lyophilate comprises a lyophilized powder. In some embodiments, the lyophilate comprises a lyophilized cake.

In some aspects, the disclosure provides extracellular vesicles (EVs) from Prevotella histicola bacteria.

In some aspects, the disclosure provides a therapeutic composition comprising the Prevotella histicola EVs, wherein the composition further comprises a pharmaceutically acceptable excipient.

In some aspects, the disclosure provides a solution comprising extracellular vesicles (EVs) from Prevotella histicola bacteria and an excipient that comprises a bulking agent.

In some aspects, the disclosure provides a solution consisting essentially of extracellular vesicles (EVs) from Prevotella histicola bacteria and an excipient that comprises a bulking agent.

In some aspects, the disclosure provides a solution comprising extracellular vesicles (EVs) from Prevotella histicola bacteria and an excipient that comprises a bulking agent and a lyoprotectant.

In some aspects, the disclosure provides a solution consisting essentially of extracellular vesicles (EVs) from Prevotella histicola bacteria and an excipient that comprises a bulking agent and a lyoprotectant.

In some aspects, the disclosure provides a solution comprising extracellular vesicles (EVs) from Prevotella histicola bacteria and an excipient that comprises a lyoprotectant.

In some aspects, the disclosure provides a solution consisting essentially of extracellular vesicles (EVs) from Prevotella histicola bacteria and an excipient that comprises a lyoprotectant.

In some aspects, the disclosure provides a therapeutic composition comprising the solution, wherein the composition further comprises a pharmaceutically acceptable excipient. In some embodiments, the pharmaceutically acceptable excipient comprises a glidant, lubricant, and/or diluent.

In some aspects, the disclosure provides a dried form comprising extracellular vesicles (EVs) from Prevotella histicola bacteria and an excipient that comprises a bulking agent.

In some aspects, the disclosure provides a dried form consisting essentially of extracellular vesicles (EVs) from Prevotella histicola bacteria and an excipient that comprises a bulking agent.

In some aspects, the disclosure provides a dried form comprising extracellular vesicles (EVs) from Prevotella histicola bacteria and an excipient that comprises a bulking agent and a lyoprotectant.

In some aspects, the disclosure provides a dried form consisting essentially of extracellular vesicles (EVs) from Prevotella histicola bacteria and an excipient that comprises a bulking agent and a lyoprotectant.

In some aspects, the disclosure provides a dried form comprising extracellular vesicles (EVs) from Prevotella histicola bacteria and an excipient that comprises a lyoprotectant.

In some aspects, the disclosure provides a dried form consisting essentially of extracellular vesicles (EVs) from Prevotella histicola bacteria and an excipient that comprises a lyoprotectant.

In some aspects, the disclosure provides a therapeutic composition comprising the dried form, wherein the composition further comprises a pharmaceutically acceptable excipient. In some embodiments, the pharmaceutically acceptable excipient comprises a glidant, lubricant, and/or diluent.

In some aspects, the disclosure provides a powder comprising extracellular vesicles (EVs) from Prevotella histicola bacteria and an excipient that comprises a bulking agent.

In some aspects, the disclosure provides a powder consisting essentially of extracellular vesicles (EVs) from Prevotella histicola bacteria and an excipient that comprises a bulking agent.

In some aspects, the disclosure provides a powder comprising extracellular vesicles (EVs) from Prevotella histicola bacteria and an excipient that comprises a bulking agent and a lyoprotectant.

In some aspects, the disclosure provides a powder consisting essentially of extracellular vesicles (EVs) from Prevotella histicola bacteria and an excipient that comprises a bulking agent and a lyoprotectant.

In some aspects, the disclosure provides a powder comprising extracellular vesicles (EVs) from Prevotella histicola bacteria and an excipient that comprises a lyoprotectant.

In some aspects, the disclosure provides a powder consisting essentially of extracellular vesicles (EVs) from Prevotella histicola bacteria and an excipient that comprises a lyoprotectant.

In some aspects, the disclosure provides a therapeutic composition comprising the powder, wherein the composition further comprises a pharmaceutically acceptable excipient. In some embodiments, the pharmaceutically acceptable excipient comprises a glidant, lubricant, and/or diluent.

In some aspects, the disclosure provides a spray-dried powder comprising extracellular vesicles (EVs) from Prevotella histicola bacteria and an excipient that comprises a bulking agent.

In some aspects, the disclosure provides a spray-dried powder consisting essentially of extracellular vesicles (EVs) from Prevotella histicola bacteria and an excipient that comprises a bulking agent.

In some aspects, the disclosure provides a spray-dried powder comprising extracellular vesicles (EVs) from Prevotella histicola bacteria and an excipient that comprises a bulking agent and a lyoprotectant.

In some aspects, the disclosure provides a spray-dried powder consisting essentially of extracellular vesicles (EVs) from Prevotella histicola bacteria and an excipient that comprises a bulking agent and a lyoprotectant.

In some aspects, the disclosure provides a spray-dried powder comprising extracellular vesicles (EVs) from Prevotella histicola bacteria and an excipient that comprises a lyoprotectant.

In some aspects, the disclosure provides a spray-dried powder consisting essentially of extracellular vesicles (EVs) from Prevotella histicola bacteria and an excipient that comprises a lyoprotectant.

In some aspects, the disclosure provides a therapeutic composition comprising the spray-dried powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilate comprising extracellular vesicles (EVs) from Prevotella histicola bacteria and an excipient that comprises a bulking agent.

In some aspects, the disclosure provides a lyophilate consisting essentially of extracellular vesicles (EVs) from Prevotella histicola bacteria and an excipient that comprises a bulking agent.

In some aspects, the disclosure provides a lyophilate comprising extracellular vesicles (EVs) from Prevotella histicola bacteria and an excipient that comprises a bulking agent and a lyoprotectant.

In some aspects, the disclosure provides a lyophilate consisting essentially of extracellular vesicles (EVs) from Prevotella histicola bacteria and an excipient that comprises a bulking agent and a lyoprotectant.

In some aspects, the disclosure provides a lyophilate comprising extracellular vesicles (EVs) from Prevotella histicola bacteria and an excipient that comprises a lyoprotectant.

In some aspects, the disclosure provides a lyophilate consisting essentially of extracellular vesicles (EVs) from Prevotella histicola bacteria and an excipient that comprises a lyoprotectant.

In some aspects, the disclosure provides a therapeutic composition comprising the lyophilate, wherein the composition further comprises a pharmaceutically acceptable excipient. In some embodiments, the pharmaceutically acceptable excipient comprises a glidant, lubricant, and/or diluent.

In some aspects, the disclosure provides a lyophilized powder comprising extracellular vesicles (EVs) from Prevotella histicola bacteria and an excipient that comprises a bulking agent.

In some aspects, the disclosure provides a lyophilized powder consisting essentially of extracellular vesicles (EVs) from Prevotella histicola bacteria and an excipient that comprises a bulking agent.

In some aspects, the disclosure provides a lyophilized powder comprising extracellular vesicles (EVs) from Prevotella histicola bacteria and an excipient that comprises a bulking agent and a lyoprotectant.

In some aspects, the disclosure provides a lyophilized powder consisting essentially of extracellular vesicles (EVs) from Prevotella histicola bacteria and from an excipient that comprises a bulking agent and a lyoprotectant.

In some aspects, the disclosure provides a lyophilized powder comprising extracellular vesicles (EVs) from Prevotella histicola bacteria and an excipient that comprises a lyoprotectant.

In some aspects, the disclosure provides a lyophilized powder consisting essentially of extracellular vesicles (EVs) from Prevotella histicola bacteria and from an excipient that comprises a lyoprotectant.

In some aspects, the disclosure provides a therapeutic composition comprising the lyophilized powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilized cake comprising extracellular vesicles (EVs) from Prevotella histicola bacteria and an excipient that comprises a bulking agent.

In some aspects, the disclosure provides a lyophilized cake consisting essentially of extracellular vesicles (EVs) from Prevotella histicola bacteria and an excipient that comprises a bulking agent.

In some aspects, the disclosure provides a lyophilized cake comprising extracellular vesicles (EVs) from Prevotella histicola bacteria and an excipient that comprises a bulking agent and a lyoprotectant.

In some aspects, the disclosure provides a lyophilized cake consisting essentially of extracellular vesicles (EVs) from Prevotella histicola bacteria and an excipient that comprises a bulking agent and a lyoprotectant.

In some aspects, the disclosure provides a lyophilized cake comprising extracellular vesicles (EVs) from Prevotella histicola bacteria and an excipient that comprises a lyoprotectant.

In some aspects, the disclosure provides a lyophilized cake consisting essentially of extracellular vesicles (EVs) from Prevotella histicola bacteria and an excipient that comprises a lyoprotectant.

In some aspects, the disclosure provides a therapeutic composition comprising extracellular vesicles (EVs) from Prevotella histicola bacteria and an excipient that comprises a bulking agent.

In some aspects, the disclosure provides a therapeutic composition consisting essentially of extracellular vesicles (EVs) from Prevotella histicola bacteria and an excipient that comprises a bulking agent.

In some aspects, the disclosure provides a therapeutic composition comprising extracellular vesicles (EVs) from Prevotella histicola bacteria and an excipient that comprises a bulking agent and a lyoprotectant.

In some aspects, the disclosure provides a therapeutic composition consisting essentially of extracellular vesicles (EVs) from Prevotella histicola bacteria and an excipient that comprises a bulking agent and a lyoprotectant.

In some aspects, the disclosure provides a therapeutic composition comprising extracellular vesicles (EVs) from Prevotella histicola bacteria and an excipient that comprises a lyoprotectant.

In some aspects, the disclosure provides a therapeutic composition consisting essentially of extracellular vesicles (EVs) from Prevotella histicola bacteria and an excipient that comprises a lyoprotectant.

In some aspects, the disclosure provides a solution comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a solution consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such solution, wherein the composition further comprises a pharmaceutically acceptable excipient. In some embodiments, the pharmaceutically acceptable excipient comprises a glidant, lubricant, and/or diluent.

In some aspects, the disclosure provides a dried form comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a dried form consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such dried form, wherein the composition further comprises a pharmaceutically acceptable excipient. In some embodiments, the pharmaceutically acceptable excipient comprises a glidant, lubricant, and/or diluent.

In some aspects, the disclosure provides a powder comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a powder consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such powder, wherein the composition further comprises a pharmaceutically acceptable excipient. In some embodiments, the pharmaceutically acceptable excipient comprises a glidant, lubricant, and/or diluent.

In some aspects, the disclosure provides a spray-dried powder comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a spray-dried powder consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such spray-dried powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilate comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a lyophilate consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such lyophilate, wherein the composition further comprises a pharmaceutically acceptable excipient. In some embodiments, the pharmaceutically acceptable excipient comprises a glidant, lubricant, and/or diluent.

In some aspects, the disclosure provides a lyophilized powder comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a lyophilized powder consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such lyophilized powder, wherein the composition further comprises a pharmaceutically acceptable excipient. In some embodiments, the pharmaceutically acceptable excipient comprises a glidant, lubricant, and/or diluent.

In some aspects, the disclosure provides a lyophilized cake comprising extracellular vesicles (EVs) from Prevotella histicola bacteria and excipients of a stock comprising one or more excipients, wherein the stock comprises a formula provided in Table A, B, C, D, K, or P.

In some aspects, the disclosure provides a lyophilized cake consisting essentially of extracellular vesicles (EVs) from Prevotella histicola bacteria and excipients of a stock comprising one or more excipients, wherein the stock comprises a formula provided in Table A, B, C, D, K, or P.

In some aspects, the disclosure provides a therapeutic composition comprising such lyophilized cake, wherein the composition further comprises a pharmaceutically acceptable excipient. In some embodiments, the pharmaceutically acceptable excipient comprises a glidant, lubricant, and/or diluent.

In some aspects, the disclosure provides a method of treating a subject (for example, human) (for example, a subject in need of treatment), the method comprising:
administering to the subject Prevotella histicola EVs or a solution, dried form, or therapeutic composition described herein.

In some embodiments, Prevotella histicola EVs or a solution, dried form, or therapeutic composition provided herein is for use in treating a subject (for example, human) (for example, a subject in need of treatment).

In some aspects, the disclosure provides use of Prevotella histicola EVs or a solution, dried form, or therapeutic composition provided herein for the preparation of a medicament for treating a subject (for example, human) (for example, a subject in need of treatment).

In some embodiments of the method, solution, dried form, therapeutic composition or use provided herein, the Prevotella histicola EVs or solution, dried form, or therapeutic composition is orally administered (for example, is for oral administration).

In some embodiments of the method, solution, dried form, therapeutic composition or use provided herein, the subject is in need of treatment (and/or prevention) of an immune disease.

In some embodiments of the method, solution, dried form, therapeutic composition or use provided herein, the subject is in need of treatment (and/or prevention) of an autoimmune disease.

In some embodiments of the method, solution, dried form, therapeutic composition or use provided herein, the subject is in need of treatment (and/or prevention) of an inflammatory disease.

In some embodiments of the method, solution, dried form, therapeutic composition or use provided herein, the subject is in need of treatment (and/or prevention) of a metabolic disease.

In some embodiments of the method, solution, dried form, therapeutic composition or use provided herein, the subject is in need of treatment (and/or prevention) of dysbiosis.

In some embodiments of the method, solution, dried form, therapeutic composition or use provided herein, the solution, dried form, or therapeutic composition is administered in combination with an additional therapeutic agent.

In some embodiments of the method, solution, dried form, therapeutic composition or use provided herein, the dried form is a powder. In some embodiments, the powder is a lyophilized powder. In some embodiments, the powder is a spray-dried powder.

In some embodiments of the method, solution, dried form, therapeutic composition or use provided herein, the dried form is a lyophilate. In some embodiments, the lyophilate is a lyophilized powder. In some embodiments, the lyophilate is a lyophilized cake.

In some aspects, the disclosure provides a method of preparing a solution that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent, thereby preparing the solution.

In some aspects, the disclosure provides a method of preparing a solution that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant, thereby preparing the solution.

In some aspects, the disclosure provides a method of preparing a solution that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant, thereby preparing the solution.

In some embodiments, the disclosure provides a solution prepared by a method described herein.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
drying the solution to prepare a cake, and milling (for example, grinding) the cake, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
drying the solution to prepare a cake, and milling (for example, grinding) the cake, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) lyoprotectant to prepare a solution;
drying the solution to prepare a cake, and milling (for example, grinding) the cake, thereby preparing the dried form.

In some embodiments of the methods of preparing a dried form provided herein, the drying comprises lyophilization.

In some embodiments of the methods of preparing a dried form provided herein, the drying comprises spray drying.

In some embodiments of the methods of preparing a dried form provided herein, the method further comprises combining the dried form with an additional ingredient. In some embodiments, the additional ingredient comprises an excipient, for example, a glidant, lubricant, and/or diluent.

In some embodiments, the disclosure provides a dried form prepared by a method described herein.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
drying the solution to prepare a cake, and milling (for example, grinding) the cake, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
drying the solution to prepare a cake, and milling (for example, grinding) the cake, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
drying the solution to prepare a cake, and milling (for example, grinding) the cake, thereby preparing the powder.

In some embodiments of the methods of preparing a powder provided herein, the drying comprises lyophilization.

In some embodiments of the methods of preparing a powder provided herein, the drying comprises spray drying.

In some embodiments of the methods of preparing a powder provided herein, the method further comprises combining the powder with an additional ingredient. In some embodiments, the additional ingredient comprises an excipient, for example, a glidant, lubricant, and/or diluent.

In some embodiments, the disclosure provides a powder prepared by a method described herein.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and spray drying the solution, thereby preparing the spray-dried powder.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and spray drying the solution, thereby preparing the spray-dried powder.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and spray drying the solution, thereby preparing the spray-dried powder.

In some embodiments of the methods of preparing a spray-dried powder provided herein, the method further comprises combining the spray-dried powder with an additional ingredient. In some embodiments, the additional ingredient comprises an excipient, for example, a glidant, lubricant, and/or diluent.

In some embodiments, the disclosure provides a spray-dried powder prepared by a method described herein.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
freeze drying (lyophilizing) the solution to prepare a cake, and milling (for example, grinding) the cake, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
freeze drying (lyophilizing) the solution to prepare a cake, and milling (for example, grinding) the cake, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
freeze drying (lyophilizing) the solution to prepare a cake, and milling (for example, grinding) the cake, thereby preparing the lyophilate.

In some embodiments of the methods of preparing a lyophilate provided herein, the method further comprises combining the lyophilate with an additional ingredient. In some embodiments, the additional ingredient comprises an excipient, for example, a glidant, lubricant, and/or diluent.

In some embodiments, the disclosure provides a lyophilate prepared by a method described herein.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
freeze drying (lyophilizing) the solution to prepare a cake, and milling (for example, grinding) the cake, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
freeze drying (lyophilizing) the solution to prepare a cake, and milling (for example, grinding) the cake, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
freeze drying (lyophilizing) the solution to prepare a cake, and
milling (for example, grinding) the cake, thereby preparing the lyophilized powder.

In some embodiments of the methods of preparing a lyophilized powder provided herein, the method further comprises combining the lyophilized powder with an additional ingredient. In some embodiments, the additional ingredient comprises an excipient, for example, a glidant, lubricant, and/or diluent.

In some embodiments, the disclosure provides a lyophilized powder prepared by a method described herein.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
freeze drying (lyophilizing) the solution, thereby preparing the lyophilized cake.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
freeze drying (lyophilizing) the solution, thereby preparing the lyophilized cake.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilized cake.

In some embodiments, the disclosure provides a lyophilized cake prepared by a method described herein.

In some aspects, the disclosure provides a method of preparing a solution that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution.

In some embodiments, the disclosure provides a solution prepared by a method described herein.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
drying the solution to prepare a cake, and milling (for example, grinding) the cake, thereby preparing the dried form.

In some embodiments of the methods of preparing a dried form provided herein, the drying comprises lyophilization.

In some embodiments of the methods of preparing a dried form provided herein, the drying comprises spray drying.

In some embodiments of the methods of preparing a dried form provided herein, the method further comprises combining the dried form with an additional ingredient. In some embodiments, the additional ingredient comprises an excipient, for example, a glidant, lubricant, and/or diluent.

In some embodiments, the disclosure provides a dried form prepared by a method described herein.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
drying the solution to prepare a cake, and milling (for example, grinding) the cake, thereby preparing the powder.

In some embodiments of the methods of preparing a powder provided herein, the drying comprises lyophilization.

In some embodiments of the methods of preparing a powder provided herein, the drying comprises spray drying.

In some embodiments of the methods of preparing a powder provided herein, the method further comprises combining the powder with an additional ingredient. In some embodiments, the additional ingredient comprises an excipient, for example, a glidant, lubricant, and/or diluent.

In some embodiments, the disclosure provides a powder prepared by a method described herein.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
spray drying the solution, thereby preparing the spray-dried powder.

In some embodiments of the methods of preparing a spray-dried powder provided herein, the method further comprises combining the spray-dried powder with an additional ingredient. In some embodiments, the additional ingredient comprises an excipient, for example, a glidant, lubricant, and/or diluent.

In some embodiments, the disclosure provides a spray-dried powder prepared by a method described herein.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
freeze drying (lyophilizing) the solution to prepare a cake, and
milling (for example, grinding) the cake, thereby preparing the lyophilate.

In some embodiments of the methods of preparing a lyophilate provided herein, the method further comprises combining the lyophilate with an additional ingredient. In some embodiments, the additional ingredient comprises an excipient, for example, a glidant, lubricant, and/or diluent.

In some embodiments, the disclosure provides a lyophilate prepared by a method described herein.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
freeze drying (lyophilizing) the solution to prepare a cake, and milling (for example, grinding) the cake, thereby preparing the lyophilized powder.

In some embodiments of the methods of preparing a lyophilized powder provided herein, the method further comprises combining the lyophilized powder with an additional ingredient. In some embodiments, the additional ingredient comprises an excipient, for example, a glidant, lubricant, and/or diluent.

In some embodiments, the disclosure provides a lyophilized powder prepared by a method described herein.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing a lyophilized cake.

In some embodiments, the disclosure provides a lyophilized cake prepared by a method described herein.

In some embodiments, the disclosure provides a lyophilized cake prepared by a method described herein.

In some embodiments of the methods that comprise a freeze drying step, the freeze drying comprises primary drying and secondary drying. In some embodiments, primary drying is performed at a temperature between about −35° C. to about −20° C. For example, primary drying is performed at a temperature of about −20° C., about −25° C., about −30° C. or about −35° C. In some embodiments, secondary drying is performed at a temperature between about +20° C. to about +30° C. For example, secondary drying is performed at a temperature of about +25° C.

In some embodiments of the solution, dried form, or therapeutic composition provided herein, the bulking agent comprises mannitol, sucrose, maltodextrin, dextran, Ficoll, polyethylene glycol (PEG, such as PEG 6000), cyclodextrin, or PVP-K30.

In some embodiments of the solution, dried form, or therapeutic composition provided herein, the bulking agent comprises mannitol.

In some embodiments of the solution, dried form, or therapeutic composition provided herein, the excipient comprises an additional ingredient.

In some embodiments of the solution, dried form, or therapeutic composition provided herein, the additional ingredient comprises trehalose, mannitol, sucrose, sorbitol, dextran, poloxamer 188, maltodextrin, PVP-K30, Ficoll, citrate, arginine, and/or hydroxypropyl-β-cyclodextrin.

In some embodiments of the solution, dried form, or therapeutic composition provided herein, the excipient comprises mannitol and trehalose.

In some embodiments of the solution, dried form, or therapeutic composition provided herein, the excipient consists essentially of mannitol and trehalose.

In some embodiments of the solution, dried form, or therapeutic composition provided herein, the excipient comprises mannitol, trehalose, and sorbitol.

In some embodiments of the solution, dried form, or therapeutic composition provided herein, the excipient consists essentially of mannitol, trehalose, and sorbitol.

In some embodiments of the solution, dried form, or therapeutic composition provided herein, the excipient comprises trehalose.

In some embodiments of the solution, dried form, or therapeutic composition provided herein, the excipient consists essentially of trehalose.

In some embodiments of the solution, dried form, or therapeutic composition provided herein, the excipient is from a stock comprising one or more excipients, wherein the stock comprises a formula provided in provided in Table A, B, C, D, K, or P.

In some embodiments of the solution, dried form, or therapeutic composition provided herein, the dried form is a powder. In some embodiments, the powder is a lyophilized powder. In some embodiments, the powder is a spray-dried powder.

In some embodiments of the solution, dried form, or therapeutic composition provided herein, the dried form is a lyophilate. In some embodiments, the lyophilate is a lyophilized powder. In some embodiments, the lyophilate is a lyophilized cake.

In some embodiments of the solution, dried form, or therapeutic composition provided herein, the excipient solution comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient solution comprises more mannitol than trehalose, for example, on a weight basis or weight percent basis. In some embodiments, the excipient solution comprises at least two-fold more mannitol than trehalose, for example, on a weight basis or weight percent basis. In some embodiments, the excipient solution comprises at least three-fold more mannitol than trehalose, for example, on a weight basis or weight percent basis. In some embodiments, the excipient of the solution or dried form comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient of the solution or dried form comprises more mannitol than trehalose, for example, on a weight basis or weight percent basis. In some embodiments, the excipient of the solution or dried form comprises at least two-fold more mannitol than trehalose, for example, on a weight basis or weight percent basis. In some embodiments, the excipient of the solution or dried form comprises at least three-fold more mannitol than trehalose, for example, on a weight basis or weight percent basis.

In some embodiments of the solution, dried form, or therapeutic composition provided herein, the excipient solution consists essentially of mannitol and trehalose. In some embodiments, the excipient solution consists essentially of mannitol and trehalose, wherein the mannitol and the trehalose are not present in equal amounts (for example, the mannitol and the trehalose are present in unequal amounts; for example, on a weight basis or a weight percent basis). In some embodiments, the excipient solution consists essentially of mannitol and trehalose, wherein the excipient contains more mannitol than trehalose, for example, on a weight basis or weight percent basis. In some embodiments, the excipient solution consists essentially of mannitol and trehalose, wherein the excipient solution contains at least two-fold more mannitol than trehalose, for example, on a weight basis or weight percent basis. In some embodiments, the excipient solution consists essentially of mannitol and trehalose, wherein the excipient solution contains at least three-fold more mannitol than trehalose, for example, on a weight basis or weight percent basis. In some embodiments, the excipient of the solution or dried form consists essentially of mannitol and trehalose, wherein the excipient of the solution or dried form contains more mannitol than trehalose, for example, on a weight basis or weight percent basis. In some embodiments, the excipient of the solution or dried form consists essentially of mannitol and trehalose, wherein the excipient of the solution or dried form contains at least two-fold more mannitol than trehalose, for example, on a weight basis or weight percent basis. In some embodiments, the excipient of the solution or dried form consists essentially of mannitol and trehalose, wherein the excipient of the solution or dried form contains at least three-fold more mannitol than trehalose, for example, on a weight basis or weight percent basis.

In some embodiments of the solution, dried form, or therapeutic composition provided herein, the excipient solution comprises, or consists essentially of, mannitol and trehalose, wherein neither the mannitol nor the trehalose is present in an amount of 5 mg/ml to 15 mg/ml. In some embodiments, the excipient solution comprises, or consists essentially of, mannitol and trehalose, wherein the mannitol is not present in an amount of 5 mg/ml to 15 mg/ml. In some embodiments, the excipient solution comprises, or consists essentially of, mannitol and trehalose, wherein the trehalose is not present in an amount of 5 mg/ml to 15 mg/ml.

In some embodiments of the solution, dried form, or therapeutic composition provided herein, the excipient solution comprises, or consists essentially of, mannitol and trehalose, wherein neither the mannitol nor the trehalose is present in an amount of 9 mg/ml. In some embodiments, the excipient solution comprises, or consists essentially of, mannitol and trehalose, wherein the mannitol is not present in an amount of 9 mg/ml. In some embodiments, the excipient solution comprises, or consists essentially of, mannitol and trehalose, wherein the trehalose is not present in an amount of 9 mg/ml.

In some embodiments of the solution, dried form, or therapeutic composition provided herein, the excipient comprises, or consists essentially of, mannitol and trehalose, and does not comprise methionine.

In some embodiments of the dried form or therapeutic composition provided herein, the dried form or therapeutic composition comprises, or consists essentially of, mannitol and trehalose, and the mannitol and the trehalose are not present in equal amounts (for example, the mannitol and the trehalose are present in unequal amounts, for example, on a weight basis or a weight percent basis) in the dried form or therapeutic composition.

In some embodiments of the solution, dried form, or therapeutic composition provided herein, at least about 10% (by weight) of the solution or dried form is excipient stock.

In some embodiments of the solution, dried form, or therapeutic composition provided herein, about 10% to about 80% (by weight) of the solution or dried form is excipient stock.

In some embodiments of the solution, dried form, or therapeutic composition provided herein, wherein about 20% to about 70% (by weight) of the solution or dried form is excipient stock.

In some embodiments of the solution, dried form, or therapeutic composition provided herein, about 30% to about 60% (by weight) of the solution or dried form is excipient stock.

In some embodiments of the dried form or therapeutic composition provided herein, the EVs from Prevotella histicola bacteria comprise at least about 1% of the total solids by weight of the dried form.

In some embodiments of the dried form or therapeutic composition provided herein, the EVs from Prevotella histicola bacteria comprise about 1% to about 99% of the total solids by weight of the dried form.

In some embodiments of the dried form or therapeutic composition provided herein, the EVs from Prevotella histicola bacteria comprise about 5% to about 90% of the total solids by weight of the dried form. In some embodiments of the dried form or therapeutic composition provided herein, the EVs from Prevotella histicola bacteria comprise about 1% to about 60% of the total solids by weight of the dried form. In some embodiments of the dried form or therapeutic composition provided herein, the EVs from Prevotella histicola bacteria comprise about 1% to about 20% of the total solids by weight of the powder or cake. In some embodiments of the dried form or therapeutic composition provided herein, the EVs from Prevotella histicola bacteria comprise about 2% to about 10% of the total solids by weight of the dried form. In some embodiments of the dried form or therapeutic composition provided herein, the EVs from Prevotella histicola bacteria comprise about 2% to about 6% of the total solids by weight of the dried form. In some embodiments of the dried form or therapeutic composition provided herein, the dried form comprises a moisture content below about 6% (for example, as determined by Karl Fischer titration).

In some embodiments of the dried form or therapeutic composition provided herein, the dried form comprises a moisture content below about 5% (for example, as determined by Karl Fischer titration).

In some embodiments of the dried form or therapeutic composition provided herein, the dried form comprises a moisture content about 0.5% to about 5% (for example, as determined by Karl Fischer titration).

In some embodiments of the dried form or therapeutic composition provided herein, the dried form comprises a moisture content about 1% to about 5% (for example, as determined by Karl Fischer titration).

In some embodiments of the dried form or therapeutic composition provided herein, the dried form comprises a moisture content about 1% to about 4% (for example, as determined by Karl Fischer titration).

In some embodiments of the dried form or therapeutic composition provided herein, the dried form comprises a moisture content about 2% to about 5% (for example, as determined by Karl Fischer titration).

In some embodiments of the dried form or therapeutic composition provided herein, the dried form comprises a moisture content about 2% to about 4% (for example, as determined by Karl Fischer titration).

In some embodiments of the dried form or therapeutic composition provided herein, the dried form comprises at least 1e10 particles per mg of the dried form (for example, as determined by particles per mg, such as by NTA).

In some embodiments of the dried form or therapeutic composition provided herein, the dried form comprises about 3e10 to about 6.5e10 particles per mg of the dried form (for example, as determined by particles per mg, such as by NTA).

In some embodiments of the dried form or therapeutic composition provided herein, the dried form comprises about 3e10 to about 8e10 particles per mg of the dried form (for example, as determined by particles per mg, such as by NTA).

In some embodiments of the dried form or therapeutic composition provided herein, the dried form comprises about 6e10 to about 8e10 particles per mg of the dried form (for example, as determined by particles per mg, such as by NTA).

In some embodiments of the dried form or therapeutic composition provided herein, the dried form comprises about 6.7e8 to about 2.55e10 particles/mg dried form (for example, as determined by particles per mg, such as by NTA).

In some embodiments of the dried form or therapeutic composition provided herein, the dried form comprises about 6.7e8 to about 2.89e10 particles/mg dried form.

In some embodiments, particle numeration is determined on a dried form by NTA. In some embodiments, particle numeration is determined on a dried form by NTA with use of a Zetaview camera.

In some embodiments, particle numeration is determined on dried form resuspended in water, by NTA and with use of a Zetaview camera.

In some embodiments of the dried form or therapeutic composition provided herein, the particles have a hydrodynamic diameter (Z average, Zₐᵥₑ) of about 100 nm to about 300 nm after resuspension from the dried form (for example, resuspension in deionized water) (for example, as determined by dynamic light scattering).

In some embodiments of the dried form or therapeutic composition provided herein, the particles have a hydrodynamic diameter (Z average, Zₐᵥₑ) of about 130 nm to about 250 nm after resuspension from the dried form (for example, resuspension in deionized water) (for example, as determined by dynamic light scattering).

In some embodiments of the dried form or therapeutic composition provided herein, the particles have a hydrodynamic diameter (Z average, Zₐᵥₑ) of about 200 nm after resuspension from the dried form (for example, resuspension in deionized water) (for example, as determined by dynamic light scattering).

In some embodiments, a solution, dried form, or therapeutic composition provided herein can contain EVs from one or more bacterial strain in addition to EVs from Prevotella histicola. In some embodiments, a solution, dried form, or therapeutic composition provided herein can contain EVs from one bacterial strain in addition to EVs from Prevotella histicola. The bacterial strain used as a source of EVs may be selected based on the properties of the bacteria (e.g., growth characteristics, yield, ability to modulate an immune response in an assay or a subject).

In some embodiments, Prevotella histicola EVs or a solution, dried form, or therapeutic composition provided herein comprising EVs from Prevotella histicola bacteria can be used for the treatment or prevention of a disease and/or a health disorder, e.g., in a subject (e.g., human).

In some embodiments, a dried form (or a therapeutic composition thereof) provided herein comprising EVs from Prevotella histicola bacteria can be prepared as a solid dose form, such as a tablet, a minitablet, a capsule, or a powder; or a combination of these forms (e.g., minitablets comprised in a capsule). The solid dose form can comprise a coating (e.g., enteric coating).

In certain embodiments, the therapeutic composition comprises a solid dose form. In some embodiments, the therapeutic composition comprises a blend of freeze-dried powder of EVs from Prevotella histicola bacteria and excipients (e.g., an encapsulated freeze-dried powder of the EVs from Prevotella histicola bacteria provided herein and excipients). In some embodiments, the therapeutic composition comprises freeze-dried (e.g., lyophilized) powder of EVs from Prevotella histicola bacteria in a capsule. In some embodiments, the capsule comprises gelatin or hydroxyl propyl methyl cellulose HPMC. In some embodiments, the capsule is enteric coated. In some embodiments, the excipients include one or more of mannitol, magnesium stearate and colloidal silicon dioxide. In some embodiments, the excipients include mannitol, magnesium stearate and colloidal silicon dioxide. In some embodiments, the therapeutic composition comprises freeze-dried (e.g., lyophilized) powder of EVs from Prevotella histicola bacteria in a tablet or mini-tablet. In some embodiments, the tablet or mini-tablet is enteric coated. In some embodiments, the excipients include one or more of silicified microcrystalline cellulose, crospovidone, magnesium stearate and colloidal silicon dioxide. In some embodiments, the excipients include silicified microcrystalline cellulose, crospovidone, magnesium stearate and colloidal silicon dioxide.

In some embodiments, a dried form (or a therapeutic composition thereof) provided herein comprising EVs from Prevotella histicola bacteria can be reconstituted. In some embodiments, a solution (or a therapeutic composition thereof) provided herein comprising EVs from Prevotella histicola bacteria can be used as suspension, e.g., diluted to a suspension or used in undiluted form.

In some embodiments, a therapeutic composition comprising Prevotella histicola EVs or a solution and/or dried form comprising EVs from Prevotella histicola bacteria can be prepared as provided herein. The therapeutic composition comprising a dried form can be formulated into a solid dose form, such as a tablet, a minitablet, a capsule, or a powder; or can be reconstituted in a suspension.

In some embodiments, Prevotella histicola EVs or a solution, dried form, or therapeutic composition provided herein can comprise gamma irradiated EVs from Prevotella histicola bacteria. The gamma irradiated EVs from Prevotella histicola bacteria can be formulated into a therapeutic composition. The gamma irradiated EVs from Prevotella histicola bacteria can be formulated into a solid dose form, such as a tablet, a minitablet, a capsule, or a powder; or can be reconstituted in a suspension.

In some embodiments, Prevotella histicola EVs or a solution, dried form, or therapeutic composition provided herein comprising EVs from Prevotella histicola bacteria can be orally administered.

In some embodiments, Prevotella histicola EVs or a solution, dried form, or therapeutic composition provided herein comprising EVs from Prevotella histicola bacteria can be administered intranasally.

In some embodiments, Prevotella histicola EVs or a solution, dried form, or therapeutic composition provided herein comprising EVs from Prevotella histicola bacteria can be administered by inhalation.

In some embodiments, Prevotella histicola EVs or a solution, dried form, or therapeutic composition provided herein comprising EVs from Prevotella histicola bacteria can be administered intravenously.

In some embodiments, Prevotella histicola EVs or a solution, dried form, or therapeutic composition provided herein comprising EVs from Prevotella histicola bacteria can be administered by injection.

In certain aspects, provided herein are therapeutic compositions comprising Prevotella histicola EVs and/or solutions and/or dried forms comprising EVs from Prevotella histicola bacteria useful for the treatment and/or prevention of a disease or a health disorder (e.g., adverse health disorders) (e.g., an immune disorder (e.g., an autoimmune disease, an inflammatory disease, an allergy), a dysbiosis, or a metabolic disease), as well as methods of making and/or identifying such Prevotella histicola EVs and/or solutions and/or dried forms and therapeutic compositions, and methods of using such Prevotella histicola EVs and/or solutions and/or dried forms, and therapeutic compositions thereof (e.g., for the treatment of an immune disorder (e.g., an autoimmune disease, an inflammatory disease, an allergy), a dysbiosis, or a metabolic disease, either alone or in combination with other therapeutics).

In some embodiments, the therapeutic compositions can comprise both EVs from Prevotella histicola bacteria and whole bacteria, e.g., Prevotella histicola bacteria from which the EVs were obtained, such as live bacteria, killed bacteria, attenuated bacteria. In some embodiments, the therapeutic compositions comprise EVs from Prevotella histicola bacteria in the absence of the bacteria from which they were obtained, such that over about 85%, over about 90%, or over about 95% (or over about 99%) of the bacteria-sourced content of the solutions and/or dried forms comprises Prevotella histicola EVs. The Prevotella histicola EVs can be isolated EVs, e.g., isolated by a method described herein.

In some embodiments, the Prevotella histicola EVs or solution, dried form, or therapeutic composition comprises isolated Prevotella histicola EVs (e.g., from one or more strains of bacteria (e.g., a therapeutically effective amount thereof). E.g., wherein at least 50%, at least 75%, at least 80%, at least 85%, at least 90%, at least 95%, or at least 99% of the content (e.g., of the content that does not exclude excipient) of the Prevotella histicola EVs and/or solution and/or dried form is isolated EVs from Prevotella histicola bacteria (e.g., bacteria of interest).

In some embodiments, the Prevotella histicola EVs or solution, dried form, or therapeutic composition comprises isolated Prevotella histicola EVs (e.g., from one strain of bacteria (e.g., bacteria of interest) (e.g., a therapeutically effective amount thereof). E.g., wherein at least 50%, at least 75%, at least 80%, at least 85%, at least 90%, at least 95%, or at least 99% of the content (e.g., of the content that does not exclude excipient) of the Prevotella histicola EVs and/or solution and/or dried form is isolated EV of Prevotella histicola bacteria (e.g., bacteria of interest, e.g., bacteria disclosed herein).

In some embodiments, the Prevotella histicola EVs or solution, dried form or therapeutic composition comprises EVs from Prevotella histicola bacteria.

In some embodiments, the solution, dried form, or therapeutic composition comprises EVs from more than one strain of bacteria (e.g., EVs from a strain in addition to the Prevotella histicola EVs).

In some embodiments, the Prevotella histicola EVs are lyophilized.

In some embodiments, the Prevotella histicola EVs are gamma irradiated.

In some embodiments, the Prevotella histicola EVs are UV irradiated.

In some embodiments, the Prevotella histicola EVs are heat inactivated (e.g., at 50° C. for two hours or at 90° C. for two hours).

In some embodiments, the Prevotella histicola EVs are acid treated.

In some embodiments, the Prevotella histicola EVs are oxygen sparged (e.g., at 0.1 vvm for two hours).

In some embodiments, the Prevotella histicola EVs are from a strain comprising at least 90% (or at least 97%) genomic, 16S and/or CRISPR sequence identity to the nucleotide sequence of the Prevotella Strain B 50329 (NRRL accession number B 50329). In some embodiments, the Prevotella histicola EVs are from a strain comprising at least 99% genomic, 16S and/or CRISPR sequence identity to the nucleotide sequence of the Prevotella Strain B 50329 (NRRL accession number B 50329). In some embodiments, the Prevotella histicola bacteria are from Prevotella Strain B 50329 (NRRL accession number B 50329).

In certain aspects, the Prevotella histicola EVs obtained from bacteria that have been selected based on certain desirable properties, such as reduced toxicity and adverse effects (e.g., by removing or deleting lipopolysaccharide (LPS)), enhanced oral delivery (e.g., by improving acid resistance, muco-adherence and/or penetration and/or resistance to bile acids, resistance to anti-bacterial peptides and/or antibody neutralization), target desired cell types (e.g., M-cells, goblet cells, enterocytes, dendritic cells, macrophages), improved bioavailability systemically or in an appropriate niche (e.g., mesenteric lymph nodes, Peyer's patches, Lamina propria, lymph nodes, and/or blood), enhanced immunomodulatory and/or therapeutic effect (e.g., either alone or in combination with another therapeutic agent), enhanced immune activation, and/or manufacturing attributes (e.g., growth characteristics, yield, greater stability, improved freeze-thaw tolerance, shorter generation times).

In certain aspects, the Prevotella histicola EVs are from engineered bacteria that are modified to enhance certain desirable properties. In some embodiments, the engineered bacteria are modified so that EVs produced therefrom will have reduced toxicity and adverse effects (e.g., by removing or deleting lipopolysaccharide (LPS)), enhanced oral delivery (e.g., by improving acid resistance, muco-adherence and/or penetration and/or resistance to bile acids, resistance to anti-microbial peptides and/or antibody neutralization), target desired cell types (e.g., M-cells, goblet cells, enterocytes, dendritic cells, macrophages), improved bioavailability systemically or in an appropriate niche (e.g., mesenteric lymph nodes, Peyer's patches, Lamina propria, lymph nodes, and/or blood), enhanced immunomodulatory and/or therapeutic effect (e.g., either alone or in combination with another therapeutic agent), enhanced immune activation, and/or improved manufacturing attributes (e.g., growth characteristics, yield, greater stability, improved freeze-thaw tolerance, shorter generation times). In some embodiments, provided herein are methods of making such EVs.

In certain aspects, provided herein are Prevotella histicola EVs and/or solutions and/or dried forms (or therapeutic compositions thereof) comprising EVs from Prevotella histicola bacteria useful for the treatment and/or prevention of a disease or a health disorder (e.g., an immune disorder (e.g., an autoimmune disease, an inflammatory disease, an allergy), a dysbiosis, or a metabolic disease), as well as methods of making and/or identifying such solutions and/or dried forms (or therapeutic compositions thereof), and methods of using such solutions and/or dried forms (e.g., for the treatment of an immune disorder (e.g., an autoimmune disease, an inflammatory disease, an allergy), a dysbiosis, or a metabolic disease), either alone or in combination with one or more other therapeutics.

Therapeutic compositions containing Prevotella histicola EVs and/or a solution and/or dried form can provide potency comparable to or greater than therapeutic compositions that contain the whole Prevotella histicola bacteria from which the EVs were obtained. For example, at the same dose of EVs (e.g., based on particle count or protein content), a therapeutic composition containing solutions and/or dried form can provide potency comparable to or greater than a comparable therapeutic composition that contains whole bacteria of the same Prevotella histicola bacterial strain from which the EVs were obtained. Such EV—and/or solution—and/or dried form-containing therapeutic compositions can allow the administration of higher doses and elicit a comparable or greater (e.g., more effective) response than observed with a comparable therapeutic composition that contains whole bacteria of the same Prevotella histicola bacterial strain from which the EVs were obtained.

As a further example, at the same dose (e.g., based on particle count or protein content), a therapeutic composition containing Prevotella histicola EVs and/or a solution and/or dried form can contain less microbially-derived material (based on particle count or protein content), as compared to a therapeutic composition that contains the whole Prevotella histicola bacteria of the same bacterial strain from which the EVs were obtained, while providing an equivalent or greater therapeutic benefit to the subject receiving such therapeutic composition.

As a further example, EVs from Prevotella histicola bacteria can be administered at doses e.g., of about 1×10⁷ to about 1×10¹⁵ particles, e.g., as measured by NTA. In some embodiments, the dose of EVs is about 1×10⁵ to about 7×10¹³ particles (e.g., wherein particle count is determined by NTA (nanoparticle tracking analysis)). In some embodiments, the dose of EVs from Prevotella histicola bacteria is about 1×10¹⁰ to about 7×10¹³ particles (e.g., wherein particle count is determined by NTA (nanoparticle tracking analysis)). NTA can be performed with Zetaview.

As another example, EVs from Prevotella histicola bacteria can be administered at doses e.g., of about 5 mg to about 900 mg total protein, e.g., as measured by Bradford assay. As another example, EVs from Prevotella histicola bacteria can be administered at doses e.g., of about 5 mg to about 900 mg total protein, e.g., as measured by BCA assay.

In certain embodiments, provided herein are methods of treating a subject who has an immune disorder (e.g., an autoimmune disease, an inflammatory disease, an allergy) comprising administering to the subject a therapeutic composition or Prevotella histicola EVs and/or a solution and/or dried form described herein. In certain embodiments, provided herein are methods of treating a subject who has a metabolic disease comprising administering to the subject a therapeutic composition or Prevotella histicola EVs and/or a solution and/or dried form described herein. In certain embodiments, provided herein are methods of treating a subject who has a dysbiosis comprising administering to the subject a therapeutic composition or Prevotella histicola EVs and/or a solution and/or dried form described herein. In certain embodiments, provided herein are methods of treating a subject who has a neurologic disease comprising administering to the subject a therapeutic composition or Prevotella histicola EVs and/or a solution and/or dried form described herein.

In some embodiments, the method further comprises administering to the subject an antibiotic. In some embodiments, the method further comprises the administration of an immune suppressant and/or an anti-inflammatory agent. In some embodiments, the therapeutic composition or Prevotella histicola EVs and/or a solution, dried form, and/or lyophilate can be for use in combination with one or more other immune effect modulators. In some embodiments, the method further comprises the administration of a metabolic disease therapeutic agent.

In certain aspects, provided herein is a therapeutic composition or Prevotella histicola EVs and/or a solution and/or dried form for use in the treatment and/or prevention of a disease (e.g., an immune disorder (e.g., an autoimmune disease, an inflammatory disease, an allergy), a dysbiosis, or a metabolic disease) or a health disorder, either alone or in combination with one or more other (e.g., additional) therapeutic agent.

In certain embodiments, provided herein is a therapeutic composition or Prevotella histicola EVs and/or a solution and/or dried form for use in treating and/or preventing an immune disorder (e.g., an autoimmune disease, an inflammatory disease, an allergy) in a subject (e.g., human). The therapeutic composition or Prevotella histicola EVs and/or a solution and/or dried form can be used either alone or in combination with one or more other therapeutic agent for the treatment of the immune disorder. In certain embodiments, provided herein is a therapeutic composition or Prevotella histicola EVs and/or a solution and/or dried form for use in treating and/or preventing a dysbiosis in a subject (e.g., human). The therapeutic composition or Prevotella histicola EVs and/or a solution and/or dried form can be used either alone or in combination with therapeutic agent for the treatment of the dysbiosis. In certain embodiments, provided herein is a therapeutic composition or Prevotella histicola EVs and/or a solution and/or dried form for use in treating and/or preventing a metabolic disease in a subject (e.g., human). The therapeutic composition or Prevotella histicola EVs and/or a solution and/or dried form can be used either alone or in combination with therapeutic agent for the treatment of the metabolic disease. In certain embodiments, provided herein is a therapeutic composition or Prevotella histicola EVs and/or a solution and/or dried form for use in treating and/or preventing a dysbiosis in a subject (e.g., human). The therapeutic composition or Prevotella histicola EVs and/or a solution and/or dried form can be used either alone or in combination with therapeutic agent for the treatment of the dysbiosis. In certain embodiments, provided herein is a therapeutic composition or Prevotella histicola EVs and/or a solution and/or dried form for use in treating and/or preventing a neurologic disease in a subject (e.g., human). The therapeutic composition or Prevotella histicola EVs and/or a solution and/or dried form can be used either alone or in combination with one or more other therapeutic agent for treatment of the neurologic disorder.

In some embodiments, the therapeutic composition or Prevotella histicola EVs and/or a solution and/or dried form can be for use in combination with an antibiotic. In some embodiments, the therapeutic composition or Prevotella histicola EVs and/or a solution and/or dried form can be for use in combination with another therapeutic bacterium and/or EVs obtained from one or more other bacterial strains (e.g., therapeutic bacterium). In some embodiments, the therapeutic composition or Prevotella histicola EVs and/or a solution and/or dried form can be for use in combination with one or more immune suppressant(s) and/or an anti-inflammatory agent(s). In some embodiments, the therapeutic composition or Prevotella histicola EVs and/or a solution and/or dried form can be for use in combination with one or more other metabolic disease therapeutic agents.

In certain aspects, provided herein is use of a therapeutic composition or Prevotella histicola EVs and/or a solution and/or dried form for the preparation of a medicament for the treatment and/or prevention of a disease (e.g., an immune disorder (e.g., an autoimmune disease, an inflammatory disease, an allergy), a dysbiosis, or a metabolic disease), either alone or in combination with another therapeutic agent. In some embodiments, the use is in combination with another therapeutic bacterium and/or EVs obtained from one or more other bacterial strains (e.g., therapeutic bacterium).

In certain embodiments, provided herein is use of a therapeutic composition or Prevotella histicola EVs and/or a solution and/or dried form (for the preparation of a medicament for treating and/or preventing an immune disorder (e.g., an autoimmune disease, an inflammatory disease, an allergy) in a subject (e.g., human). The therapeutic composition or Prevotella histicola EVs and/or a solution and/or dried form can be for use either alone or in combination with another therapeutic agent for the immune disorder. In certain embodiments, provided herein is use of a therapeutic composition or Prevotella histicola EVs and/or a solution and/or dried form for the preparation of a medicament for treating and/or preventing a dysbiosis in a subject (e.g., human). The therapeutic composition or Prevotella histicola EVs and/or a solution and/or dried form can be for use either alone or in combination with another therapeutic agent for the dysbiosis. In certain embodiments, provided herein is use of a therapeutic composition or Prevotella histicola EVs and/or a solution and/or dried form for the preparation of a medicament for treating and/or preventing a metabolic disease in a subject (e.g., human). The therapeutic composition or Prevotella histicola EVs and/or a solution and/or dried form can be for use either alone or in combination with another therapeutic agent for the metabolic disease. In certain embodiments, provided herein is use of a therapeutic composition or Prevotella histicola EVs and/or a solution and/or dried form for the preparation of a medicament for treating and/or preventing a dysbiosis in a subject (e.g., human). The therapeutic composition or Prevotella histicola EVs and/or a solution and/or dried form can be for use either alone or in combination with another therapeutic agent for the dysbiosis. In certain embodiments, provided herein is use of a therapeutic composition or Prevotella histicola EVs and/or a solution and/or dried form for the preparation of a medicament for treating and or preventing a neurologic disease in a subject (e.g., human). The therapeutic composition or Prevotella histicola EVs and/or a solution and/or dried form can be for use either alone or in combination with another therapeutic agent for the neurologic disorder.

In some embodiments, the therapeutic composition or Prevotella histicola EVs and/or a solution and/or dried form can be for use in combination with an antibiotic. In some embodiments, the therapeutic composition or Prevotella histicola EVs and/or a solution and/or dried form can be for use in combination with another therapeutic bacterium and/or EVs obtained from one or more other bacterial strains (e.g., therapeutic bacterium). In some embodiments, the therapeutic composition or Prevotella histicola EVs and/or a solution and/or dried form can be for use in combination with one or more other immune suppressant(s) and/or an anti-inflammatory agent(s). In some embodiments, the therapeutic composition or Prevotella histicola EVs and/or a solution and/or dried form can be for use in combination with one or more other metabolic disease therapeutic agent(s).

A therapeutic composition or Prevotella histicola EVs and/or a solution and/or dried form, e.g., as described herein, comprising EVs from Prevotella histicola bacteria can provide a therapeutically effective amount of Prevotella histicola EVs to a subject, e.g., a human.

A therapeutic composition or Prevotella histicola EVs and/or a solution and/or dried form, e.g., as described herein, comprising EVs from Prevotella histicola bacteria can provide a non-natural amount of the therapeutically effective components (e.g., present in the Prevotella histicola EVs to a subject, e.g., a human.

A therapeutic composition or Prevotella histicola EVs and/or a solution and/or dried form, e.g., as described herein, comprising EVs from Prevotella histicola bacteria can provide unnatural quantity of the therapeutically effective components (e.g., present in the EVs to a subject, e.g., a human.

A therapeutic composition or Prevotella histicola EVs and/or a solution and/or dried form, e.g., as described herein, comprising EVs from Prevotella histicola bacteria can bring about one or more changes to a subject, e.g., human, e.g., to treat or prevent a disease or a health disorder.

A therapeutic composition or Prevotella histicola EVs and/or a solution and/or dried form, e.g., as described herein, comprising EVs from Prevotella histicola bacteria has potential for significant utility, e.g., to affect a subject, e.g., a human, e.g., to treat or prevent a disease or a health disorder.

In certain aspects, provided herein is a stock comprising one or more excipients, wherein the stock comprises a bulking agent, wherein the stock is for use in combination with extracellular vesicles (EVs) from Prevotella histicola bacteria (for example, a liquid preparation thereof).

In certain aspects, provided herein is a stock comprising one or more excipients, wherein the stock comprises a bulking agent and a lyoprotectant, wherein the stock is for use in combination with extracellular vesicles (EVs) from Prevotella histicola bacteria (for example, a liquid preparation thereof).

In certain aspects, provided herein is a stock comprising one or more excipients, wherein the stock comprises a lyoprotectant, wherein the stock is for use in combination with extracellular vesicles (EVs) from Prevotella histicola bacteria (for example, a liquid preparation thereof).

In some embodiments, the bulking agent comprises mannitol, sucrose, maltodextrin, dextran, Ficoll, or PVP-K30.

In some embodiments, the bulking agent comprises mannitol.

In some embodiments, the excipient solution comprises an additional ingredient.

In some embodiments, the additional ingredient comprises trehalose, mannitol, sucrose, sorbitol, dextran, poloxamer 188, maltodextrin, PVP-K30, Ficoll, citrate, arginine, and/or hydroxypropyl-B-cyclodextrin.

In some embodiments, the excipient solution comprises mannitol and trehalose.

In some embodiments, the excipient solution consists essentially of mannitol and trehalose.

In some embodiments, the excipient solution comprises mannitol, trehalose, and sorbitol.

In some embodiments, the excipient solution consists essentially of mannitol, trehalose, and sorbitol.

In some embodiments, the excipient solution comprises trehalose.

In some embodiments, the excipient solution consists essentially of trehalose.

In some embodiments, the excipient solution comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient solution comprises more mannitol than trehalose, for example, on a weight basis or weight percent basis. In some embodiments, the excipient solution comprises at least two-fold more mannitol than trehalose, for example, on a weight basis or weight percent basis. In some embodiments, the excipient solution comprises at least three-fold more mannitol than trehalose, for example, on a weight basis or weight percent basis. In some embodiments, the excipient of the solution or dried form comprises mannitol and trehalose, wherein the mannitol and the trehalose are not present in equal amounts (for example, the mannitol and the trehalose are present in unequal amounts; for example, on a weight basis or a weight percent basis). In some embodiments, the excipient of the solution or dried form comprises more mannitol than trehalose, for example, on a weight basis or weight percent basis. In some embodiments, the excipient of the solution or dried form comprises at least two-fold more mannitol than trehalose, for example, on a weight basis or weight percent basis. In some embodiments, the excipient of the solution or dried form comprises at least three-fold more mannitol than trehalose, for example, on a weight basis or weight percent basis.

In some embodiments, the excipient solution consists essentially of mannitol and trehalose. In some embodiments, the excipient solution consists essentially of mannitol and trehalose, wherein the mannitol and the trehalose are not present in equal amounts (for example, the mannitol and the trehalose are present in unequal amounts; for example, on a weight basis or a weight percent basis). In some embodiments, the excipient solution consists essentially of mannitol and trehalose, wherein the excipient solution contains more mannitol than trehalose, for example, on a weight basis or weight percent basis. In some embodiments, the excipient solution consists essentially of mannitol and trehalose, wherein the excipient solution contains at least two-fold more mannitol than trehalose, for example, on a weight basis or weight percent basis. In some embodiments, the excipient solution consists essentially of mannitol and trehalose, wherein the excipient solution contains at least three-fold more mannitol than trehalose, for example, on a weight basis or weight percent basis. In some embodiments, the excipient of the solution or dried form consists essentially of mannitol and trehalose, wherein the excipient of the solution or dried form contains more mannitol than trehalose, for example, on a weight basis or weight percent basis. In some embodiments, the excipient of the solution or dried form consists essentially of mannitol and trehalose, wherein the excipient of the solution or dried form contains at least two-fold more mannitol than trehalose, for example, on a weight basis or weight percent basis. In some embodiments, the excipient of the solution or dried form consists essentially of mannitol and trehalose, wherein the excipient of the solution or dried form contains at least three-fold more mannitol than trehalose, for example, on a weight basis or weight percent basis.

In some embodiments, the excipient solution comprises, or consists essentially of, mannitol and trehalose, wherein neither the mannitol nor the trehalose is present in an amount of 5 mg/ml to 15 mg/ml. In some embodiments, the excipient solution comprises, or consists essentially of, mannitol and trehalose, wherein the mannitol is not present in an amount of 5 mg/ml to 15 mg/ml. In some embodiments, the excipient solution comprises, or consists essentially of, mannitol and trehalose, wherein the trehalose is not present in an amount of 5 mg/ml to 15 mg/ml.

In some embodiments, the excipient solution comprises, or consists essentially of, mannitol and trehalose, wherein neither the mannitol nor the trehalose is present in an amount of 9 mg/ml. In some embodiments, the excipient solution comprises, or consists essentially of, mannitol and trehalose, wherein the mannitol is not present in an amount of 9 mg/ml. In some embodiments, the excipient solution comprises, or consists essentially of, mannitol and trehalose, wherein the trehalose is not present in an amount of 9 mg/ml.

In some embodiments, the excipient solution comprises, or consists essentially of, mannitol and trehalose, and does not comprise methionine.

In certain aspects, provided herein is a stock comprising one or more excipients, wherein the stock comprises a formula provided in Table A, B, C, D, K, or P.

In certain aspects, provided herein is a stock comprising one or more excipients, wherein the stock comprises a formula provided in Table A, B, C, D, K, or P wherein the stock is for use in combination with extracellular vesicles (EVs) from Prevotella histicola bacteria (for example, a liquid preparation thereof).

In some embodiments of the solutions and dried forms and methods described herein, a liquid preparation comprises a cell culture supernatant, such as a bacterial cell culture supernatant, for example, as described herein. In some embodiments of the solution and dried forms and methods described herein, the liquid preparation comprises a retentate, such as a concentrated retentate, for example, as described herein.

In some embodiments of the methods provided herein, excipients are present in (for example, provided in) an excipient solution. Examples of an excipient solution include the stocks comprising one or more excipients provided in Tables A, B, C, D, K, or P. For example, the dried forms provided herein contain excipients from the excipient solution (such as a stock) once the moisture has been removed, such as by drying. For example, a liquid preparation that comprises EVs from Prevotella histicola bacteria is combined with the stock of formula 7a (which comprises the excipients mannitol and trehalose) from Table A to prepare a solution. The solution is dried to prepare a dried form. The dried form comprises EVs from Prevotella histicola bacteria, mannitol, and trehalose. As used herein, a “stock” refers to a solution comprising one or more excipients but no active ingredient (such as an extracellular vesicle). In some embodiments, a stock is used to introduce one or more excipients into a preparation (such as a liquid preparation) comprising EVs. In some embodiments, the stock is a concentrated solution comprising a known amount of one or more excipients. In some embodiments, the stock is combined with a preparation (such as a liquid preparation) that comprises EVs to prepare a solution or dried form provided herein.

### BRIEF DESCRIPTION OF THE FIGURES

FIGS. 1A and 1B are graphs showing that orally-administered Prevotella EVs require multiple pathways for anti-inflammatory effects in a delayed type hypersensitivity (DTH) model of inflammation. Inflammation is assessed as change in ear thickness (mm). The effects of antibodies against TLR2 (anti-TLR2) or IL-10 receptor (IL-10R) (anti-IL-10R) (FIG. 1A), and of antibodies against CD62 and LPAM-1 (anti-CD62/LPAM-1) (FIG. 1B) on the ability of Prevotella EVs to reduce inflammation were assessed. Dexamethasone was used as a positive control for inflammation reduction.

FIG. 2 is a graph showing Prevotella EVs have potent human TLR2 agonist activity in a HEK293 reporter cell assay. TLR1/2/6, TLR1/2 or TLR2/6 activation at various concentrations of Prevotella EVs was determined as a normalized response (OD630 nm).

FIG. 3 is a graph showing TLR1/2 signaling is required to induce IL-10 release from U937 cells in response Prevotella EVs. The effects of antibodies against TLR2 (anti-TLR2), TLR1 (anti-TLR-1), and TLR6 (anti-TLR6) on the ability of Prevotella EVs to cause IL-10 cytokine release (pg/ml) from U937 cells was measured.

FIGS. 4A and 4B are graphs showing Prevotella EVs induce IL-10 (FIG. 4A) and IL-27 (FIG. 4B) concentration-dependent production from human PBMCs. IL-10 and IL-27 levels were measured as pg/ml at various concentrations of Prevotella EVs.

FIG. 5 is a graph showing the effects of orally-administered Prevotella EVs powder prepared in formula 7a in a delayed type hypersensitivity (DTH) model of inflammation. Inflammation is assessed as change in ear thickness (mm).

FIG. 6 is a graph showing the effects of orally-administered Prevotella smEVs (EVs) and intraperitoneally-administered anti-TNFa antibody in a delayed type hypersensitivity (DTH) model of inflammation. Inflammation is assessed as change in ear thickness (mm).

FIG. 7 is a graph showing changes in ear thickness 24 hours after KLH ear challenge following dosing with vehicle (PBS), dexamethasone, or Prevotella smEVs (EVs) or smEVs (EVs) from another bacterial strain (other strain EVs) either before (pre) or after (post) immunization with KLH-CFA.

FIG. 8 is a graph showing changes in ear thickness 24 hours after KLH ear challenge following immunization with either IFA-PBS or CFA-PBS, dosing with vehicle (PBS), dexamethasone, or Prevotella smEVs (EVs), then immunization with KLH-CFA.

FIG. 9 is a graph showing changes in ear thickness 24 hours after KLH ear challenge in recipient mice following transfer of CD4+ T cells from KLH-CFA immunized donor mice dosed with vehicle or Prevotella smEVs (EVs) or smEVs (EVs) from another bacterial strain (other strain EVs).

FIG. 10 is a graph showing changes in ear thickness 24 hours after KLH ear challenge in recipient mice following transfer of CD4+ T cells from isotype control-or anti-TLR2-treated KLH-CFA immunized mice dosed with vehicle or Prevotella smEVs (EVs) into KLH-CFA immunized recipient mice.

FIG. 11 is a graph showing changes in ear thickness 24 hours after KLH ear challenge in recipient mice following transfer of CD8+ T cells from KLH-CFA immunized donor mice dosed with vehicle or Prevotella smEVs (EV) or smEVs (EVs) from another bacterial strain (other strain EVs).

### DETAILED DESCRIPTION

The disclosure provides Prevotella histicola EVs, and solutions, dried forms and therapeutic compositions that contain extracellular vesicles (EVs) from Prevotella histicola bacteria, and methods for preparing and using the same.

The small intestinal axis is a network of anatomical and functional connections linking the small intestine with the rest of the body. It senses external signals in the gut lumen and translates them into systemic immune effects. We have previously shown that an oral microbial drug candidate induces anti-inflammatory activity in preclinical models of inflammation by acting directly on host cells without colonization of the gut or modulation of the microbiome. We now extend these observations to Prevotella histicola EVs, that have potent anti-inflammatory activity in preclinical models. EVs are non-replicating bacterial membrane vesicles with approximately 1/1000th the volume of the parent cell. Prevotella histicola EVs were orally delivered and gut-restricted in distribution, they act by modulation of innate and adaptive immunity within the small intestine to attenuate systemic inflammatory responses.

The disclosure provides solutions and dried forms that contain extracellular vesicles (EVs) from Prevotella histicola bacteria, and methods for preparing and using the same. The disclosure also provides therapeutic compositions that contain the solutions and/or dried forms. In some embodiments, EVs are secreted (for example, produced) by bacterial cells in culture. Such secreted extracellular vesicles may be referred to as secreted microbial extracellular vesicles (smEVs). In some embodiments, EVs are prepared (for example, artificially prepared) by processing bacterial cells, for example, by methods that disrupt the bacterial membrane, such as sonication. Such artificially prepared may be referred to as processed microbial extracellular vesicles (pmEVs).

As used herein, a “dried form” that contains extracellular vesicles (EVs) (for example, from Prevotella histicola bacteria) refers to the product resulting from drying a solution that contains EVs. In some embodiments, the drying is performed, for example, by freeze drying (lyophilization) or spray drying. In some embodiments, the dried form is a powder. As used herein, a powder refers to a type of dried form and includes a lyophilized powder and a spray-dried powder, obtained by a method such as spray drying.

When freeze drying (lyophilization) is performed, the resulting dried form is a lyophilate. In some embodiments, the dried form is a lyophilate. For example, in some embodiments, a lyophilate is a lyophilized powder or a lyophilized cake. In some embodiments, the lyophilized cake is milled to produce a lyophilized powder.

In some embodiments, the solutions and dried forms that contain EVs from Prevotella histicola bacteria also comprise one or more excipients, such as a bulking agent, and/or a lyoprotectant.

In some embodiments, bulking agents and lyoprotectants are used when preparing extracellular vesicles (EVs) for freeze drying. In some embodiments, bulking agents, including but not limited to sucrose, mannitol, polyethylene glycol (PEG, such as PEG 6000), cyclodextrin, maltodextrin, and dextran (such as dextran 40k), are added (for example, as a stock containing the same) to a liquid preparation of EVs (for example, obtained by isolating EVs from a bacterial culture) to prepare a dried form such as a lyophilate, making it easier to handle (and optionally, further formulate, for example, into a therapeutic composition) after drying. In some embodiments, lyoprotectants, including but not limited to trehalose, sucrose, and lactose, are added (for example, as a stock containing the same) to a liquid preparation of EVs (for example, obtained by isolating EVs from a bacterial culture) to protect the EVs while lyophilizing or spray drying. In some embodiments, a bulking agent and/or lyoprotectant is included from an excipient stock that is added to EVs (for example, purified and/or concentrated EVs) to produce a solution, and/or to produce a dried form upon subsequent drying, for example, of the solution. In some embodiments, a dried form such as a lyophilate contains between about 5% and about 100% EV solids by weight. In some embodiments, prior to drying (such as by lyophilization), the total solids, including EVs and excipients, are between about 2% and about 20% by weight.

As described herein, in some embodiments, in a lyophilate containing Prevotella histicola EVs, the excipients make up about 95% to about 99% of the total mass of the powder or cake.

As described herein, in some embodiments, in a lyophilate containing Prevotella histicola EVs, the EVs make up about 2% to about 6% (for example, about 2% to about 5%, about 2% to about 3%, or about 3% to about 5%) of the total mass of the lyophilate.

In some embodiments, the excipient functions to maintain EV efficacy and/or decrease drying (for example, lyophilization) cycle time. In some embodiments, lyoprotectants protect EVs (for example, protein components thereof) during the freeze-drying process. In some embodiments, bulking agents improve the lyophilate properties, for example, for further downstream processing (such as milling, blending, and/or preparing therapeutic compositions).

The length of the lyophilization cycle is important for cost considerations. Critical temperature modifiers such as bulking agents and/or lyoprotectants can significantly reduce drying time. In some embodiments, an excipient stock containing one or more excipients (for example, that contain a bulking agent and/or lyoprotectant) is added to concentrated EVs (for example, a liquid preparation thereof) to bring the total solids to between about 2% to about 20%. In some embodiments, the EVs are concentrated to 5 to 100 times or volume concentration factors (VCF). Examples provided herein targeted about 10% total solids with actual dissolved solids ranging from about 6% to about 8%. In some embodiments, an excipient stock containing one or more excipients (for example, that contain a bulking agent and/or lyoprotectant) (for example, a stock comprising excipients of a formula provided in one of Tables A, B, C, D, K, or P) is prepared as a stock solution in deionized water and sterile filtered with a 0.2 mm filter prior to use. In some embodiments, the stock solution is added to the concentrated EVs, for example, based on weight up to 80%. In some embodiments, the percentage to add is based on the estimated solids contribution of EVs plus the dissolved solids of the excipient stock to achieve the desired total solids content prior to lyophilization.

After freeze drying Prevotella histicola EVs (for example, with an excipient that comprises a bulking agent, for example, as described herein), in some embodiments, the resulting lyophilate (for example, lyophilized cake) has a uniform appearance, and is a white to off-white. In some embodiments, the resulting lyophilate (for example, lyophilized cake) obtained after freeze-drying is a white to off-white, fine and smooth granular powder (for example, after milling (for example, grinding) the lyophilized cake). In some embodiments, dynamic light scattering (DLS) is used to obtain the hydrodynamic diameter (Z average, Zₐᵥₑ) of particles present after the lyophilate (for example, lyophilized powder) is resuspended in deionized water or in a buffer such as PBS (for example, 0.1×PBS). In some embodiments, the Zₐᵥₑ is used to quantify the effectiveness of the stabilizer. For example, if the idealized Zₐᵥₑ particle size is 200 nm; therefore, the resuspended EVs with the lowest Zₐᵥₑ closest to this particle size is considered to be sufficiently stabilized. In some embodiments, the particle size ranges, for example, from 130 nm to 300 nm. In some embodiments, dynamic light scattering (DLS) is used to obtain the mean size of the most dominant DLS integrated peak of particles present after the lyophilate (for example, lyophilized powder) is resuspended in deionized water or in a buffer such as PBS (for example, 0.1×PBS). Notably, the mean size of the particles, whether measured by Z average or by the mean size of the most dominant DLS integrated peak, is not necessarily identical to the mean size of the EVs prior to lyophilization. For example, in some embodiments, the mean size of the particles after lyophilization (for example, after the lyophilate is resuspended in deionized water or in a buffer such as PBS (for example, 0.1×PBS)) is larger or smaller than the mean EV size prior to lyophilization, or the mean size after EV isolation or preparation from a bacterial culture (for example, the mean size after gradient purification of EVs from a bacterial culture). Particles in a lyophilate (after a solution containing EVs is lyophilized) contain Prevotella histicola EVs, and may also include other components from the culture media, such as cell debris, LPS, and/or proteins.

A lyophilate obtained after freeze-drying with the excipients and/or conditions provided herein does not have a porous sponge shape. In some embodiments, after milling, the lyophilate obtained after freeze-drying with the excipients and/or conditions provided herein is a white to off-white, fine and smooth granular lyophilate powder.

Also as described herein, use of the excipients provided herein allows a solution comprising Prevotella histicola EVs to be freeze dried at higher temperatures and shorter drying times. For example, the excipients and methods provided herein allow for EVs to be freeze dried in less than 4000 minutes, for example, freeze dried in about 2800 to about 3200 minutes. As another example, in some embodiments, the freezing step is performed in less than 225 minutes, as opposed to 10 to 15 hours (600 to 900 minutes). As another example, in some embodiments, using the excipients and methods provided herein, primary drying is performed at a temperature between about −35° C. to about −20° C., for example, about −20° C., about −25° C., about −30° C. or about −35° C., as opposed to, for example, −50° C. As another example, in some embodiments, using the excipients and methods provided herein, primary drying is performed for about 42 hours or less (for example, 2500 minutes or less), as opposed to, for example, 50-60 hours (3000 to 3600 minutes). In some embodiments, using the excipients and methods provided herein, total dry times are, for example, about 72 hours or less, for example, about 48 to about 72 hours, for example, less than about 48 hours. In some embodiments, using the excipients and methods provided herein, primary drying is performed for about 65 hours or less (for example, about 60 hours or less). In some embodiments, using the excipients and methods provided herein, secondary drying is performed for about 12 hours or less (for example, about 10 to about 12 hours, about 5 to about 10 hours, about 10 hours or less, or about 5 hours or less). As another example, in some embodiments, using the excipients and methods provided herein, secondary drying is performed at a temperature between about +20° C. to about +30° C., for example, room temperature, for example, about +25° C., as opposed to, for example, −20° C. In some embodiments, use of shorter drying times and/or higher drying temperatures makes the lyophilization process for EVs more commercially feasible.

In some embodiments, the lyophilates containing Prevotella histicola EVs described herein (for example, prepared using the excipients and/or methods described herein) are prepared to have a moisture content (for example, as determined by the Karl Fischer method) of below about 10% (for example, below about 9%, below about 8%, below about 7%, below about 6%, below about 5% or below about 4%, for example, about 1% to about 4%, about 1.5% to about 4%, about 2% to about 4%, about 2% to about 3%) upon completion of freeze drying. In some embodiments, by preparing lyophilates to have a moisture content below about 6%, the lyophilate are better suited for downstream processing, for example, for use in a therapeutic composition. In some embodiments, by preparing lyophilates to have a moisture content below about 6%, the lyophilate has improved stability, e.g., upon storage.

As described in the examples provided herein, the moisture content (determined by Karl Fischer) of lyophilates containing Prevotella histicola EVs had moisture contents of between about 1.8% to about 3.8%. Components of the excipient can be selected to obtain the desired moisture content. The drying conditions can be selected to obtain the desired moisture content.

In some embodiments, the lyophilates containing Prevotella histicola EVs described herein (for example, prepared using the excipients and/or methods described herein) are prepared to have a particle numeration of about 3.25e10 to about 7.77e10 particles/mg lyophilate. In some embodiments, particle numeration is determined, for example, by NTA, on lyophilate resuspended in water and with use of a Zetaview camera. In some embodiments, the lyophilates containing Prevotella histicola EVs described herein (for example, prepared using the excipients and/or methods described herein) are prepared to have a particle numeration of about 3.25e10 to about 6.45e10 particles/mg lyophilate. In some embodiments, particle numeration is determined, for example, by NTA, on lyophilate resuspended in water and with use of a Zetaview camera. Components of the excipient can be selected to obtain the desired particle numeration. The drying conditions can be selected to obtain the desired particle numeration.

In some embodiments, the particles in the lyophilates (for example, lyophilized powders) described herein (for example, prepared using the excipients and/or methods described herein) are prepared to have a hydrodynamic diameter (Z average, Zₐᵥₑ) of about 137.4 nm to about 226.1 nm. In some embodiments, the particles in the lyophilates (for example, lyophilized powders) described herein (for example, prepared using the excipients and/or methods described herein) are prepared to have a hydrodynamic diameter (Z average, Zₐᵥₑ) of about 137.4 nm to about 212.8 nm. In some embodiments, dynamic light scattering (DLS) is used to obtain the hydrodynamic diameter (Z average, Zₐᵥₑ) of particles present after the lyophilate is resuspended in deionized water or in a buffer such as PBS (for example, 0.1×PBS). Components of the excipient can be selected to obtain the desired Zₐᵥₑ. The drying conditions can be selected to obtain the desired Zₐᵥₑ.

In some embodiments, the spray-dried powders containing EVs described herein (for example, prepared using the excipients and/or methods described herein) are prepared to have a moisture content (for example, as determined by the Karl Fischer method) of below about 10% (for example, below about 9%, below about 8%, below about 7%, below about 6%, below about 5% or below about 4%, for example, about 1% to about 4%, about 1.5% to about 4%, about 2% to about 3%) upon completion of spray drying. In some embodiments, by preparing spray-dried powders to have a moisture content below about 6%, the spray-dried powders are better suited for downstream processing, for example, for use in a therapeutic composition. In some embodiments, by preparing spray-dried powders to have a moisture content below about 6%, the spray-dried powder has improved stability, e.g., upon storage.

As described in the examples provided herein, the moisture content (determined by Karl Fischer) of spray-dried powders containing Prevotella histicola EVs had moisture contents of between about 2.54% to about 8.38%. Components of the excipient can be selected to obtain the desired moisture content. The drying conditions can be selected to obtain the desired moisture content.

In some embodiments, the spray-dried powders containing EVs described herein (for example, prepared using the excipients and/or methods described herein) are prepared to have a particle numeration of about 6.7e8 to about 2.55e10 particles/mg spray-dried powder. In some embodiments, particle numeration is determined, for example, by NTA using a Zetaview camera.

As described in the examples provided herein, spray-dried powders containing Prevotella histicola EVs had particle numerations of about 8.05e9 to about 2.e10 particles/mg spray-dried powder. Components of the excipient can be selected to obtain the desired particle numeration. The drying conditions can be selected to obtain the desired particle numeration.

### Definitions

Unless specifically stated or obvious from context, as used herein, the term “or” is understood to be inclusive. Unless specifically stated or obvious from context, as used herein, the terms “a,” “an,” and “the” are understood to be singular or plural.

“Adjuvant” or “Adjuvant therapy” broadly refers to an agent that affects an immunological or physiological response in a patient or subject (e.g., human). For example, an adjuvant might increase the presence of an antigen over time or to an area of interest like a tumor, help absorb an antigen presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent.

“Administration” broadly refers to a route of administration of a composition (e.g., a therapeutic composition) to a subject. Examples of routes of administration include oral administration, rectal administration, topical administration, inhalation (nasal) or injection. Administration by injection includes intravenous (IV), intramuscular (IM), and subcutaneous (SC) administration. A therapeutic composition described herein can be administered in any form by any effective route, including but not limited to oral, parenteral, enteral, intravenous, intraperitoneal, topical, transdermal (e.g., using any standard patch), intradermal, ophthalmic, (intra) nasally, local, non-oral, such as aerosol, inhalation, subcutaneous, intramuscular, buccal, sublingual, (trans) rectal, vaginal, intra-arterial, and intrathecal, transmucosal (e.g., sublingual, lingual, (trans) buccal, (trans) urethral, vaginal (e.g., trans- and perivaginally), implanted, intravesical, intrapulmonary, intraduodenal, intragastrical, and intrabronchial. In preferred embodiments, a therapeutic composition described herein is administered orally, rectally, topically, intravesically, by injection into or adjacent to a draining lymph node, intravenously, by inhalation or aerosol, or subcutaneously. In another preferred embodiment, a therapeutic composition described herein is administered orally or intravenously.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen binding fragment” and “antigen-binding portion” of an antibody, as used herein, refer to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES R), isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

A “carbohydrate” refers to a sugar or polymer of sugars. The terms “saccharide,” “polysaccharide,” “carbohydrate,” and “oligosaccharide” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replaced with a fluorine, or N-acetylglucosamine, a nitrogen-containing form of glucose (e.g., 2′-fluororibose, deoxyribose, and hexose). Carbohydrates may exist in many different forms, for example, conformers, cyclic forms, acyclic forms, stereoisomers, tautomers, anomers, and isomers.

“Cellular augmentation” broadly refers to the influx of cells or expansion of cells in an environment that are not substantially present in the environment prior to administration of a composition and not present in the composition itself. Cells that augment the environment include immune cells, stromal cells, bacterial and fungal cells.

“Clade” refers to the OTUs or members of a phylogenetic tree that are downstream of a statistically valid node in a phylogenetic tree. The clade comprises a set of terminal leaves in the phylogenetic tree that is a distinct monophyletic evolutionary unit and that share some extent of sequence similarity.

A “combination” can refer to EVs from one source strain with another agent, e.g., another EV (e.g., from another strain), with bacteria (e.g., of the same or different strain that the EV was obtained from), or with another therapeutic agent. The combination can be in physical co-existence, either in the same material or product or in physically connected products, as well as the temporal co-administration or co-localization of the EVs and other agent.

As used herein, the term “consists essentially of” (or “consisting essentially of”) means limited to the recited elements and/or steps and those that do not materially affect the basic and novel characteristics of the claimed invention.

“Dysbiosis” refers to a state of the microbiota or microbiome of the gut or other body area, including, e.g., mucosal or skin surfaces (or any other microbiome niche) in which the normal diversity and/or function of the host gut microbiome ecological networks (“microbiome”) are disrupted. A state of dysbiosis may result in a diseased state, or it may be unhealthy under only certain conditions or only if present for a prolonged period. Dysbiosis may be due to a variety of factors, including, environmental factors, infectious agents, host genotype, host diet and/or stress. A dysbiosis may result in: a change (e.g., increase or decrease) in the prevalence of one or more bacteria types (e.g., anaerobic), species and/or strains, change (e.g., increase or decrease) in diversity of the host microbiome population composition: a change (e.g., increase or reduction) of one or more populations of symbiont organisms resulting in a reduction or loss of one or more beneficial effects: overgrowth of one or more populations of pathogens (e.g., pathogenic bacteria); and/or the presence of, and/or overgrowth of, symbiotic organisms that cause disease only when certain conditions are present.

The term “decrease” or “deplete” means a change, such that the difference is, depending on circumstances, at least 10%, 20%, 30%, 40%, 50%, 60%, 70%, 80%, 90%, 1/100, 1/1000, 1/10,000, 1/100,000, 1/1,000,000 or undetectable after treatment when compared to a pre-treatment state. Properties that may be decreased include the number of immune cells, bacterial cells, stromal cells, myeloid derived suppressor cells, fibroblasts, metabolites: the level of a cytokine; or another physical parameter (such as ear thickness (e.g., in a DTH animal model) or tumor size (e.g., in an animal tumor model)).

The term “effective dose” is the amount of the therapeutic composition that is effective to achieve the desired therapeutic response for a particular subject, composition, and mode of administration, with the least toxicity to the subject.

As used herein, “engineered bacteria” are any bacteria that have been genetically altered from their natural state by human activities, and the progeny of any such bacteria. Engineered bacteria include, for example, the products of targeted genetic modification, the products of random mutagenesis screens and the products of directed evolution.

The term “epitope” means a protein determinant capable of specific binding to an antibody or T cell receptor. Epitopes usually consist of chemically active surface groupings of molecules such as amino acids or sugar side chains. Certain epitopes can be defined by a particular sequence of amino acids to which an antibody is capable of binding.

“Extracellular vesicles” (EVs) may be naturally-produced vesicles derived from bacteria, such as smEVs. EVs are comprised of bacterial lipids and/or bacterial proteins and/or bacterial nucleic acids and/or bacterial carbohydrate moieties, and are isolated from culture supernatant. The natural production of these vesicles can be artificially enhanced (for example, increased) or decreased through manipulation of the environment in which the bacterial cells are being cultured (for example, by media or temperature alterations). Further, EV compositions may be modified to reduce, increase, add, or remove bacterial components or foreign substances to alter efficacy, immune stimulation, stability, immune stimulatory capacity, stability, organ targeting (for example, lymph node), absorption (for example, gastrointestinal), and/or yield (for example, thereby altering the efficacy). As used herein, the term “purified EV composition” or “EV composition” refers to a preparation of EVs that have been separated from at least one associated substance found in a source material (for example, separated from at least one other bacterial component) or any material associated with the EVs in any process used to produce the preparation. It can also refer to a composition that has been significantly enriched for specific components. Extracellular vesicles may also be obtained from mammalian cells and from can be obtained from microbes such as archaea, fungi, microscopic algae, protozoans, and parasites. Extracellular vesicles from any of these sources can be prepared into a solution and/or dried form as described herein. Extracellular vesicles may be artificially-produced vesicles prepared from bacteria, such as pmEVs, for example, obtained by chemically disrupting (for example, by lysozyme and/or lysostaphin) and/or physically disrupting (for example, by mechanical force) bacterial cells and separating the bacterial membrane components from the intracellular components through centrifugation and/or ultracentrifugation, or other methods, can also be prepared into a solution and/or dried form as described herein.

The term “gene” is used broadly to refer to any nucleic acid associated with a biological function. The term “gene” applies to a specific genomic sequence, as well as to a cDNA or an mRNA encoded by that genomic sequence.

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12 (I): 387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

As used herein, the term “immune disorder” refers to any disease, disorder or disease symptom caused by an activity of the immune system, including autoimmune diseases, inflammatory diseases and allergies. Immune disorders include, but are not limited to, autoimmune diseases (e.g., psoriasis, atopic dermatitis, lupus, scleroderma, hemolytic anemia, vasculitis, type one diabetes, Grave's disease, rheumatoid arthritis, multiple sclerosis, Goodpasture's syndrome, pernicious anemia and/or myopathy), inflammatory diseases (e.g., acne vulgaris, asthma, celiac disease, chronic prostatitis, glomerulonephritis, inflammatory bowel disease, pelvic inflammatory disease, reperfusion injury, rheumatoid arthritis, sarcoidosis, transplant rejection, vasculitis and/or interstitial cystitis), and/or an allergies (e.g., food allergies, drug allergies and/or environmental allergies).

“Immunotherapy” is treatment that uses a subject's immune system to treat disease (e.g., immune disease, inflammatory disease, metabolic disease, cancer) and includes, for example, checkpoint inhibitors, cancer vaccines, cytokines, cell therapy, CAR-T cells, and dendritic cell therapy.

The term “increase” means a change, such that the difference is, depending on circumstances, at least 10%, 20%, 30%, 40%, 50%, 60%, 70%, 80%, 90%, 2-fold, 4-fold, 10-fold, 100-fold, 10\hat{ }3 fold, 10\hat{ }4 fold, 10\hat{ }5 fold, 10\hat{ }6 fold, and/or 10\hat{ }7 fold greater after treatment when compared to a pre-treatment state. Properties that may be increased include the number of immune cells, bacterial cells, stromal cells, myeloid derived suppressor cells, fibroblasts, metabolites: the level of a cytokine; or another physical parameter (such as ear thickness (e.g., in a DTH animal model) or tumor size (e.g., in an animal tumor model).

“Innate immune agonists” or “immuno-adjuvants” are small molecules, proteins, or other agents that specifically target innate immune receptors including Toll-Like Receptors (TLR), NOD receptors, RLRs, C-type lectin receptors, STING-cGAS Pathway components, inflammasome complexes. For example, LPS is a TLR-4 agonist that is bacterially derived or synthesized and aluminum can be used as an immune stimulating adjuvant. immuno-adjuvants are a specific class of broader adjuvant or adjuvant therapy. Examples of STING agonists include, but are not limited to, 2′3′-cGAMP, 3′3′-cGAMP, c-di-AMP, c-di-GMP, 2′2′-cGAMP, and 2′3′-cGAM (PS) 2 (Rp/Sp) (Rp, Sp-isomers of the bis-phosphorothioate analog of 2′3′-cGAMP). Examples of TLR agonists include, but are not limited to, TLR1, TLR2, TLR3, TLR4, TLR5, TLR6, TLR7, TLR8, TLR9, TLR10 and TLR11. Examples of NOD agonists include, but are not limited to, N-acetylmuramyl-L-alanyl-D-isoglutamine (muramyldipeptide (MDP)), gamma-D-glutamyl-meso-diaminopimelic acid (iE-DAP), and desmuramylpeptides (DMP).

The “internal transcribed spacer” or “ITS” is a piece of non-functional RNA located between structural ribosomal RNAs (rRNA) on a common precursor transcript often used for identification of eukaryotic species in particular fungi. The rRNA of fungi that forms the core of the ribosome is transcribed as a signal gene and consists of the 8S, 5.8S and 28S regions with ITS4 and 5 between the 8S and 5.8S and 5.8S and 28S regions, respectively. These two intercistronic segments between the 18S and 5.8S and 5.8S and 28S regions are removed by splicing and contain significant variation between species for barcoding purposes as previously described (Schoch et al Nuclear ribosomal internal transcribed spacer (ITS) region as a universal DNA barcode marker for Fungi. PNAS 109:6241-6246. 2012). 18S rDNA is traditionally used for phylogenetic reconstruction however the ITS can serve this function as it is generally highly conserved but contains hypervariable regions that harbor sufficient nucleotide diversity to differentiate genera and species of most fungus.

The term “isolated” or “enriched” encompasses a microbe, an EV (such as a bacterial EV) or other entity or substance that has been (1) separated from at least some of the components with which it was associated when initially produced (whether in nature or in an experimental setting), and/or (2) produced, prepared, purified, and/or manufactured by the hand of man. Isolated bacteria or EVs may be separated from at least about 10%, about 20%, about 30%, about 40%, about 50%, about 60%, about 70%, about 80%, about 90%, or more of the other components with which they were initially associated. In some embodiments, isolated bacteria or EVs are more than about 80%, about 85%, about 90%, about 91%, about 92%, about 93%, about 94%, about 95%, about 96%, about 97%, about 98%, about 99%, or more than about 99% pure, e.g., substantially free of other components.

As used herein a “lipid” includes fats, oils, triglycerides, cholesterol, phospholipids, fatty acids in any form including free fatty acids. Fats, oils and fatty acids can be saturated, unsaturated (cis or trans) or partially unsaturated (cis or trans).

“Metabolite” as used herein refers to any and all molecular compounds, compositions, molecules, ions, co-factors, catalysts or nutrients used as substrates in any cellular or bacterial metabolic reaction or resulting as product compounds, compositions, molecules, ions, co-factors, catalysts or nutrients from any cellular or bacterial metabolic reaction.

“Microbiome” broadly refers to the microbes residing on or in body site of a subject or patient. Microbes in a microbiome may include bacteria, viruses, eukaryotic microorganisms, and/or viruses. Individual microbes in a microbiome may be metabolically active, dormant, latent, or exist as spores, may exist planktonically or in biofilms, or may be present in the microbiome in sustainable or transient manner. The microbiome may be a commensal or healthy-state microbiome or a disease-state or dysbiotic microbiome. The microbiome may be native to the subject or patient, or components of the microbiome may be modulated, introduced, or depleted due to changes in health state or treatment conditions (e.g., antibiotic treatment, exposure to different microbes). In some aspects, the microbiome occurs at a mucosal surface. In some aspects, the microbiome is a gut microbiome.

A “microbiome profile” or a “microbiome signature” of a tissue or sample refers to an at least partial characterization of the bacterial makeup of a microbiome. In some embodiments, a microbiome profile indicates whether at least 2, 3, 4, 5, 6, 7, 8, 9, 10, 15, 20, 25, 30, 35, 40, 45, 50, 55, 60, 65, 70, 75, 80, 85, 90, 95, 100 or more bacterial strains are present or absent in a microbiome.

“Modified” in reference to a bacteria broadly refers to a bacteria that has undergone a change from its wild-type form. Bacterial modification can result from engineering bacteria. Examples of bacterial modifications include genetic modification, gene expression modification, phenotype modification, formulation modification, chemical modification, and dose or concentration. Examples of improved properties are described throughout this specification and include, e.g., attenuation, auxotrophy, homing, or antigenicity. Phenotype modification might include, by way of example, bacteria growth in media that modify the phenotype of a bacterium such that it increases or decreases virulence.

“Operational taxonomic units” and “OTU(s)” refer to a terminal leaf in a phylogenetic tree and is defined by a nucleic acid sequence, e.g., the entire genome, or a specific genetic sequence, and all sequences that share sequence identity to this nucleic acid sequence at the level of species. In some embodiments the specific genetic sequence may be the 16S sequence or a portion of the 16S sequence. In other embodiments, the entire genomes of two entities are sequenced and compared. In another embodiment, select regions such as multilocus sequence tags (MLST), specific genes, or sets of genes may be genetically compared. For 16S, OTUs that share ≥97% average nucleotide identity across the entire 16S or some variable region of the 16S are considered the same OTU. See e.g., Claesson M J, Wang Q, O'Sullivan O, Greene-Diniz R, Cole J R, Ross R P, and O'Toole P W. 2010. Comparison of two next-generation sequencing technologies for resolving highly complex microbiota composition using tandem variable 16S rRNA gene regions. Nucleic Acids Res 38: e200. Konstantinidis K T, Ramette A, and Tiedje J M. 2006. The bacterial species definition in the genomic era. Philos Trans R Soc Lond B Biol Sci 361:1929-1940. For complete genomes, MLSTs, specific genes, other than 16S, or sets of genes OTUs that share ≥95% average nucleotide identity are considered the same OTU. See e.g., Achtman M, and Wagner M. 2008. Microbial diversity and the genetic nature of microbial species. Nat. Rev. Microbiol. 6:431-440. Konstantinidis K T, Ramette A, and Tiedje J M. 2006. The bacterial species definition in the genomic era. Philos Trans R Soc Lond B Biol Sci 361:1929-1940. OTUs are frequently defined by comparing sequences between organisms. Generally, sequences with less than 95% sequence identity are not considered to form part of the same OTU. OTUs may also be characterized by any combination of nucleotide markers or genes, in particular highly conserved genes (e.g., “house-keeping” genes), or a combination thereof. Operational Taxonomic Units (OTUs) with taxonomic assignments made to, e.g., genus, species, and phylogenetic clade are provided herein.

As used herein, a gene is “overexpressed” in a bacteria if it is expressed at a higher level in an engineered bacteria under at least some conditions than it is expressed by a wild-type bacteria of the same species under the same conditions. Similarly, a gene is “underexpressed” in a bacteria if it is expressed at a lower level in an engineered bacteria under at least some conditions than it is expressed by a wild-type bacteria of the same species under the same conditions.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), micro RNA (miRNA), silencing RNA (siRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a substance is “pure” if it is substantially free of other components. The terms “purify,” “purifying” and “purified” refer to an EV (such as an EV from bacteria) preparation or other material that has been separated from at least some of the components with which it was associated either when initially produced or generated (e.g., whether in nature or in an experimental setting), or during any time after its initial production. An EV preparation or compositions may be considered purified if it is isolated at or after production, such as from one or more other bacterial components, and a purified microbe or bacterial population may contain other materials up to about 10%, about 20%, about 30%, about 40%, about 50%, about 60%, about 70%, about 80%, about 90%, or above about 90% and still be considered “purified.” In some embodiments, purified EVs are more than about 80%, about 85%, about 90%, about 91%, about 92%, about 93%, about 94%, about 95%, about 96%, about 97%, about 98%, about 99%, or more than about 99% pure. EV compositions (or preparations) are, e.g., purified from residual habitat products.

As used herein, the term “purified EV composition” or “EV composition” refers to a preparation that includes EVs from bacteria that have been separated from at least one associated substance found in a source material (e.g., separated from at least one other bacterial component) or any material associated with the EVs in any process used to produce the preparation. It also refers to a composition that has been significantly enriched or concentrated. In some embodiments, the EVs are concentrated by 2 fold, 3-fold, 4-fold, 5-fold, 10-fold, 100-fold, 1000-fold, 10,000-fold or more than 10,000 fold.

“Residual habitat products” refers to material derived from the habitat for microbiota within or on a subject. For example, fermentation cultures of microbes can contain contaminants, e.g., other microbe strains or forms (e.g., bacteria, virus, mycoplasm, and/or fungus). For example, microbes live in feces in the gastrointestinal tract, on the skin itself, in saliva, mucus of the respiratory tract, or secretions of the genitourinary tract (i.e., biological matter associated with the microbial community). Substantially free of residual habitat products means that the microbial composition no longer contains the biological matter associated with the microbial environment on or in the culture or human or animal subject and is 100% free, 99% free, 98% free, 97% free, 96% free, or 95% free of any contaminating biological matter associated with the microbial community. Residual habitat products can include abiotic materials (including undigested food) or it can include unwanted microorganisms. Substantially free of residual habitat products may also mean that the microbial composition contains no detectable cells from a culture contaminant or a human or animal and that only microbial cells are detectable. In one embodiment, substantially free of residual habitat products may also mean that the microbial composition contains no detectable viral (including bacteria, viruses (e.g., phage)), fungal, mycoplasmal contaminants. In another embodiment, it means that fewer than 1×10⁻²%, 1×10⁻³%, 1×10⁻⁴%, 1×10⁻⁵%, 1×10⁻⁶%, 1×10⁻⁷%, 1×10⁻⁸% of the viable cells in the microbial composition are human or animal, as compared to microbial cells. There are multiple ways to accomplish this degree of purity, none of which are limiting. Thus, contamination may be reduced by isolating desired constituents through multiple steps of streaking to single colonies on solid media until replicate (such as, but not limited to, two) streaks from serial single colonies have shown only a single colony morphology. Alternatively, reduction of contamination can be accomplished by multiple rounds of serial dilutions to single desired cells (e.g., a dilution of 10⁻⁸ or 10⁻⁹), such as through multiple 10-fold serial dilutions. This can further be confirmed by showing that multiple isolated colonies have similar cell shapes and Gram staining behavior. Other methods for confirming adequate purity include genetic analysis (e.g., PCR, DNA sequencing), serology and antigen analysis, enzymatic and metabolic analysis, and methods using instrumentation such as flow cytometry with reagents that distinguish desired constituents from contaminants.

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1,000 fold less than its affinity for binding to a non-specific and unrelated antigen/binding partner (e.g., BSA, casein). Alternatively, specific binding applies more broadly to a two component system where one component is a protein, lipid, or carbohydrate or combination thereof and engages with the second component which is a protein, lipid, carbohydrate or combination thereof in a specific way.

“Strain” refers to a member of a bacterial species with a genetic signature such that it may be differentiated from closely-related members of the same bacterial species. The genetic signature may be the absence of all or part of at least one gene, the absence of all or part of at least on regulatory region (e.g., a promoter, a terminator, a riboswitch, a ribosome binding site), the absence (“curing”) of at least one native plasmid, the presence of at least one recombinant gene, the presence of at least one mutated gene, the presence of at least one foreign gene (a gene derived from another species), the presence at least one mutated regulatory region (e.g., a promoter, a terminator, a riboswitch, a ribosome binding site), the presence of at least one non-native plasmid, the presence of at least one antibiotic resistance cassette, or a combination thereof. Genetic signatures between different strains may be identified by PCR amplification optionally followed by DNA sequencing of the genomic region(s) of interest or of the whole genome. In the case in which one strain (compared with another of the same species) has gained or lost antibiotic resistance or gained or lost a biosynthetic capability (such as an auxotrophic strain), strains may be differentiated by selection or counter-selection using an antibiotic or nutrient/metabolite, respectively.

The terms “subject” or “patient” refers to any mammal. A subject or a patient described as “in need thereof” refers to one in need of a treatment (or prevention) for a disease. Mammals (i.e., mammalian animals) include humans, laboratory animals (e.g., primates, rats, mice), livestock (e.g., cows, sheep, goats, pigs), and household pets (e.g., dogs, cats, rodents). The subject may be a human. The subject may be a non-human mammal including but not limited to of a dog, a cat, a cow, a horse, a pig, a donkey, a goat, a camel, a mouse, a rat, a guinea pig, a sheep, a llama, a monkey, a gorilla or a chimpanzee. The subject may be healthy, or may be suffering from a disease or disorder at any developmental stage.

As used herein, the term “therapeutic agent” refers to an agent for therapeutic use. In some embodiments, a therapeutic agent is a composition comprising EVs (“an EV composition”) that can be used to treat and/or prevent a disease and/or condition. In some embodiments, the therapeutic agent is a pharmaceutical agent. In some embodiments, a medicinal product, medical food, a food product, or a dietary supplement comprises a therapeutic agent. In some embodiments, the therapeutic agent is in a solution, and in other embodiments, a dried form. The dried form embodiments may be produced, for example, by lyophilization or spray drying. In some embodiments, the dried form of the therapeutic agent is a lyophilized cake or powder. In some embodiments, the dried form of the therapeutic agent is a spray-dried powder.

As used herein, the term “therapeutic composition” or “pharmaceutical composition” refers to a composition that comprises a therapeutically effective amount of a therapeutic agent (for example an EV composition described herein). In some embodiments, the therapeutic composition is (or is present in) a medicinal product, medical food, a food product, or a dietary supplement.

As used herein, the term “treating” a disease in a subject or “treating” a subject having or suspected of having a disease refers to administering to the subject to a pharmaceutical treatment, e.g., the administration of one or more agents, such that at least one symptom of the disease is decreased or prevented from worsening. Thus, in one embodiment, “treating” refers inter alia to delaying progression, expediting remission, inducing remission, augmenting remission, speeding recovery, increasing efficacy of or decreasing resistance to alternative therapeutics, or a combination thereof. As used herein, the term “preventing” a disease in a subject refers to administering to the subject to a pharmaceutical treatment, e.g., the administration of one or more agents, such that onset of at least one symptom of the disease is delayed or prevented.

### Bacteria

In certain aspects, provided herein are Prevotella histicola extracellular vesicles (EVs), and solutions and/or dried forms, and therapeutic compositions, that comprise Prevotella histicola extracellular vesicles (EVs). In certain aspects, provided herein are solutions and/or dried forms, and therapeutic compositions, that comprise EVs obtained from Prevotella histicola bacteria.

In some embodiments, Prevotella histicola bacteria from which EVs are obtained are lyophilized.

In some embodiments, Prevotella histicola bacteria from which EVs are obtained are gamma irradiated (e.g., at 17.5 or 25 kGy).

In some embodiments, Prevotella histicola bacteria from which EVs are obtained are UV irradiated.

In some embodiments, Prevotella histicola bacteria from which EVs are obtained are heat inactivated (e.g., at 50° C. for two hours or at 90° C. for two hours).

In some embodiments, Prevotella histicola bacteria from which EVs are obtained are acid treated.

In some embodiments, Prevotella histicola bacteria from which EVs are obtained are oxygen sparged (e.g., at 0.1 vvm for two hours).

In some embodiments, the Prevotella histicola EVs are lyophilized.

In some embodiments, the Prevotella histicola EVs are spray dried.

In some embodiments, the Prevotella histicola EVs are gamma irradiated (e.g., at 17.5 or 25 kGy).

In some embodiments, the Prevotella histicola EVs are UV irradiated.

In some embodiments, the Prevotella histicola EVs are heat inactivated (e.g., at 50° C. for two hours or at 90° C. for two hours).

In some embodiments, the Prevotella histicola EVs are acid treated.

In some embodiments, the Prevotella histicola EVs are oxygen sparged (e.g., at 0.1 vvm for two hours).

The phase of growth can affect the amount or properties of bacteria and/or EVs produced by Prevotella histicola bacteria. For example, in the methods of EVs preparation provided herein, EVs can be isolated, e.g., from a culture, at the start of the log phase of growth, midway through the log phase, and/or once stationary phase growth has been reached.

In some embodiments, the Prevotella histicola EVs are from one strain of bacteria, e.g., a strain provided herein.

In some embodiments, the Prevotella histicola EVs are from one strain of bacteria (e.g., a strain provided herein) or from more than one strain.

In some embodiments, the EVs are from Prevotella histicola bacteria, e.g., from a strain comprising at least 90% or at least 99% genomic, 16S and/or CRISPR sequence identity to the nucleotide sequence of the Prevotella Strain B 50329 (NRRL accession number B 50329). In some embodiments, the EVs are from Prevotella histicola bacteria, e.g., from Prevotella Strain B 50329 (NRRL accession number B 50329).

In some embodiments, the Prevotella histicola bacteria from which the EVs are obtained are modified (e.g., engineered) to reduce toxicity or other adverse effects, to enhance delivery) (e.g., oral delivery) of the EVs (e.g., by improving acid resistance, muco-adherence and/or penetration and/or resistance to bile acids, digestive enzymes, resistance to anti-microbial peptides and/or antibody neutralization), to target desired cell types (e.g., M-cells, goblet cells, enterocytes, dendritic cells, macrophages), to enhance their immunomodulatory and/or therapeutic effect of the EVs (e.g., either alone or in combination with another therapeutic agent), and/or to enhance immune activation or suppression by the EVs (e.g., through modified production of polysaccharides, pili, fimbriae, adhesins). In some embodiments, the engineered bacteria described herein are modified to improve EV manufacturing (e.g., higher oxygen tolerance, stability, improved freeze-thaw tolerance, shorter generation times). For example, in some embodiments, the engineered bacteria described include bacteria harboring one or more genetic changes, such change being an insertion, deletion, translocation, or substitution, or any combination thereof, of one or more nucleotides contained on the bacterial chromosome or endogenous plasmid and/or one or more foreign plasmids, wherein the genetic change may results in the overexpression and/or underexpression of one or more genes. The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, or any combination thereof.

### Modified EVs

In some aspects, the Prevotella histicola EVs described herein are modified such that they comprise, are linked to, and/or are bound by a therapeutic moiety.

In some embodiments, the Prevotella histicola EVs described herein are engineered such that they comprise, are linked to, and/or are bound by a magnetic and/or paramagnetic moiety (e.g., a magnetic bead). In some embodiments, the magnetic and/or paramagnetic moiety is comprised by and/or directly linked to the bacteria. In some embodiments, the magnetic and/or paramagnetic moiety is linked to and/or a part of an EV-binding moiety that that binds to the EV. In some embodiments, the EV-binding moiety is a fragment of or a full-length peptidoglycan recognition protein, such as PGRP.

In some embodiments the EV-binding moiety has binding specificity for the EV (e.g., by having binding specificity for a bacterial antigen). In some embodiments, the EV-binding moiety comprises an antibody or antigen binding fragment thereof. In some embodiments, the EV-binding moiety comprises a T cell receptor or a chimeric antigen receptor (CAR).

### Production of Bacterial Extracellular Vesicles (EVs)

Secreted EVs. In certain aspects, the EVs (such as secreted EVs (smEVs) from bacteria described herein) are prepared using any method known in the art.

In some embodiments, the smEVs are prepared without an smEV purification step. For example, in some embodiments, bacteria described herein are killed using a method that leaves the smEVs intact and the resulting bacterial components, including the smEVs, are used in the methods and compositions described herein. In some embodiments, the bacteria are killed using an antibiotic (for example, using an antibiotic described herein). In some embodiments, the bacteria are killed using UV irradiation. In some embodiments, the bacteria are heat-killed.

In some embodiments, the smEVs described herein are purified from one or more other bacterial components. Methods for purifying smEVs from bacteria are known in the art. In some embodiments, smEVs are prepared from bacterial cultures using methods described in S. Bin Park, et al. PLoS ONE. 6 (3): e17629 (2011) or G. Norheim, et al. PLoS ONE. 10 (9): e0134353 (2015) or Jeppesen, et al. Cell 177:428 (2019), each of which is hereby incorporated by reference in its entirety. In some embodiments, the bacteria are cultured to high optical density and then centrifuged to pellet bacteria (for example, at 10,000×g for 30 min at 4° C., at 15,500×g for 15 min at 4° C.). In some embodiments, the culture supernatants are then passed through filters to exclude intact bacterial cells (for example, a 0.22 μm filter). In some embodiments, the supernatants are then subjected to tangential flow filtration, during which the supernatant is concentrated, species smaller than 100 kDa are removed, and the media is partially exchanged with PBS. In some embodiments, filtered supernatants are centrifuged to pellet bacterial smEVs (for example, at 100,000-150,000×g for 1-3 hours at 4° C., at 200,000×g for 1-3 hours at 4° C.). In some embodiments, the smEVs are further purified by resuspending the resulting smEV pellets (for example, in PBS), and applying the resuspended smEVs to an Optiprep (iodixanol) gradient or gradient (for example, a 30-60% discontinuous gradient, a 0-45% discontinuous gradient), followed by centrifugation (for example, at 200,000×g for 4-20 hours at 4° C.). smEV bands can be collected, diluted with PBS, and centrifuged to pellet the smEVs (for example, at 150,000×g for 3 hours at 4° C., at 200,000×g for 1 hour at 4° C.). The purified smEVs can be stored, for example, at −80° C. or −20° C. until use. In some embodiments, the smEVs are further purified by treatment with DNase and/or proteinase K.

For example, in some embodiments, cultures of bacteria can be centrifuged at 11,000×g for 20-40 min at 4° C. to pellet bacteria. Culture supernatants may be passed through a 0.22 μm filter to exclude intact bacterial cells. Filtered supernatants may then be concentrated using methods that may include, but are not limited to, ammonium sulfate precipitation, ultracentrifugation, or filtration. For example, for ammonium sulfate precipitation, 1.5-3 M ammonium sulfate can be added to filtered supernatant slowly, while stirring at 4° C. Precipitations can be incubated at 4° C. for 8-48 hours and then centrifuged at 11,000×g for 20-40 min at 4° C. The resulting pellets contain bacteria smEVs and other debris. Using ultracentrifugation, filtered supernatants can be centrifuged at 100,000-200,000×g for 1-16 hours at 4° C. The pellet of this centrifugation contains bacterial smEVs and other debris such as large protein complexes. In some embodiments, using a filtration technique, such as through the use of an Amicon Ultra spin filter or by tangential flow filtration, supernatants can be filtered so as to retain species of molecular weight >50 or 100 kDa.

Alternatively, smEVs can be obtained from bacteria cultures continuously during growth, or at selected time points during growth, for example, by connecting a bioreactor to an alternating tangential flow (ATF) system (for example, XCell ATF from Repligen). The ATF system retains intact cells (>0.22 μm) in the bioreactor, and allows smaller components (for example, smEVs, free proteins) to pass through a filter for collection. For example, the system may be configured so that the <0.22 μm filtrate is then passed through a second filter of 100 kDa, allowing species such as smEVs between 0.22 μm and 100 kDa to be collected, and species smaller than 100 kDa to be pumped back into the bioreactor. Alternatively, the system may be configured to allow for medium in the bioreactor to be replenished and/or modified during growth of the culture. smEVs collected by this method may be further purified and/or concentrated by ultracentrifugation or filtration as described above for filtered supernatants.

smEVs obtained by methods provided herein may be further purified by size-based column chromatography, by affinity chromatography, by ion-exchange chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in PBS and 3 volumes of 60% Optiprep are added to the sample. In some embodiments, if filtration was used to concentrate the filtered supernatant, the concentrate is diluted using 60% Optiprep to a final concentration of 35% Optiprep. Samples are applied to a 0-45% discontinuous Optiprep gradient and centrifuged at 200,000×g for 3-24 hours at 4° C., for example, 4-24 hours at 4° C.

In some embodiments, to confirm sterility and isolation of the smEV preparations, smEVs are serially diluted onto agar medium used for routine culture of the bacteria being tested, and incubated using routine conditions. Non-sterile preparations are passed through a 0.22 μm filter to exclude intact cells. To further increase purity, isolated smEVs may be DNase or proteinase K treated.

In some embodiments, for preparation of smEVs used for in vivo injections, purified smEVs are processed as described previously (G. Norheim, et al. PLoS ONE. 10 (9): e0134353 (2015)). Briefly, after sucrose gradient centrifugation, bands containing smEVs are resuspended to a final concentration of 50 μg/mL in a solution containing 3% sucrose or other solution suitable for in vivo injection known to one skilled in the art. This solution may also contain adjuvant, for example aluminum hydroxide at a concentration of 0-0.5% (w/v). In some embodiments, for preparation of smEVs used for in vivo injections, smEVs in PBS are sterile-filtered to <0.22 μm.

In certain embodiments, to make samples compatible with further testing (for example, to remove sucrose prior to TEM imaging or in vitro assays), samples are buffer exchanged into PBS or 30 mM Tris, pH 8.0 using filtration (for example, Amicon Ultra columns), dialysis, or ultracentrifugation (200,000×g, ≥3 hours, 4° C.) and resuspension.

In some embodiments, the sterility of the smEV preparations can be confirmed by plating a portion of the smEVs onto agar medium used for standard culture of the bacteria used in the generation of the smEVs and incubating using standard conditions.

In some embodiments, select smEVs are isolated and enriched by chromatography and binding surface moieties on smEVs. In other embodiments, select smEVs are isolated and/or enriched by fluorescent cell sorting by methods using affinity reagents, chemical dyes, recombinant proteins or other methods known to one skilled in the art.

In some embodiments, smEVs are analyzed, for example, as described in Jeppesen, et al. Cell 177:428 (2019).

In some embodiments, smEVs are lyophilized.

In some embodiments, smEVs are spray dried.

In some embodiments, smEVs are gamma irradiated (for example, at 17.5 or 25 kGy).

In some embodiments, smEVs are UV irradiated.

In some embodiments, smEVs are heat inactivated (for example, at 50° C. for two hours or at 90° C. for two hours).

In some embodiments, smEVs are acid treated.

In some embodiments, smEVs are oxygen sparged (for example, at 0.1 vvm for two hours).

The phase of growth can affect the amount or properties of bacteria and/or smEVs produced by bacteria. For example, in the methods of smEV preparation provided herein, smEVs can be isolated, for example, from a culture, at the start of the log phase of growth, midway through the log phase, and/or once stationary phase growth has been reached.

The growth environment (for example, culture conditions) can affect the amount of smEVs produced by bacteria. For example, the yield of smEVs can be increased by an smEV inducer, as provided in Table 4.

TABLE 4
Culture Techniques to Increase smEV Production

| smEV | | |
|---|---|---|
| inducement | smEV inducer | Acts on |
| Temperature | Heat | stress response |
| | RT to 37° C. temp change | simulates infection |
| | 37 to 40° C. temp change | febrile infection |
| ROS | Plumbagin | oxidative stress response |
| | Cumene hydroperoxide | oxidative stress response |
| | Hydrogen Peroxide | oxidative stress response |
| Antibiotics | Ciprofloxacin | bacterial SOS response |
| | Gentamycin | protein synthesis |
| | Polymyxin B | outer membrane |
| | D-cycloserine | cell wall |
| Osmolyte | NaCl | osmotic stress |
| Metal Ion | Iron Chelation | iron levels |
| Stress | EDTA | removes divalent cations |
| | Low Hemin | iron levels |
| Media | Lactate | growth |
| additives | Amino acid deprivation | stress |
| or removal | Hexadecane | stress |
| | Glucose | growth |
| | Sodium bicarbonate | ToxT induction |
| | PQS | vesiculator (from bacteria) |
| | Diamines + DFMO | membrane anchoring |
| | | (negativicutes only) |
| | High nutrients | enhanced growth |
| | Low nutrients | |
| Other | Oxygen | oxygen stress in anaerobe |
| mechanisms | No Cysteine | oxygen stress in anaerobe |
| | Inducing biofilm or | |
| | floculation | |
| | Diauxic Growth | |
| | Phage | |
| | Urea | |

In the methods for preparing smEVs provided herein, the methods can optionally include exposing a culture of bacteria to an smEV inducer prior to isolating smEVs from the bacterial culture. The culture of bacteria can be exposed to an smEV inducer at the start of the log phase of growth, midway through the log phase, and/or once stationary phase growth has been reached.

Processed EVs. In certain aspects, the EVs (such as processed EVs (pmEVs) described herein) are prepared (for example, artificially prepared) using any method known in the art.

In some embodiments, the pmEVs are prepared without a pmEV purification step. For example, in some embodiments, bacteria from which the pmEVs described herein are released are killed using a method that leaves the bacterial pmEVs intact, and the resulting bacterial components, including the pmEVs, are used in the methods and compositions described herein. In some embodiments, the bacteria are killed using an antibiotic (for example, using an antibiotic described herein). In some embodiments, the bacteria are killed using UV irradiation.

In some embodiments, the pmEVs described herein are purified from one or more other bacterial components. Methods for purifying pmEVs from bacteria (and optionally, other bacterial components) are known in the art. In some embodiments, pmEVs are prepared from bacterial cultures using methods described in Thein et al. (J. Proteome Res. 9(12):6135-6147 (2010)) or Sandrini et al. (Bio-protocol 4(21): e1287 (2014)), each of which is hereby incorporated by reference in its entirety. In some embodiments, the bacteria are cultured to high optical density and then centrifuged to pellet bacteria (for example, at 10,000-15,000×g for 10-15 min at room temperature or 4° C.). In some embodiments, the supernatants are discarded and cell pellets are frozen at −80° C. In some embodiments, cell pellets are thawed on ice and resuspended in 100 mM Tris-HCl, pH 7.5 supplemented with 1 mg/mL DNase I. In some embodiments, cells are lysed using an Emulsiflex C-3 (Avestin, Inc.) under conditions recommended by the manufacturer. In some embodiments, debris and unlysed cells are pelleted by centrifugation at 10,000×g for 15 min at 4° C. In some embodiments, supernatants are then centrifuged at 120,000×g for 1 hour at 4° C. In some embodiments, pellets are resuspended in ice-cold 100 mM sodium carbonate, pH 11, incubated with agitation for 1 hour at 4° C., and then centrifuged at 120,000×g for 1 hour at 4° C. In some embodiments, pellets are resuspended in 100 mM Tris-HCl, pH 7.5, re-centrifuged at 120,000×g for 20 min at 4° C., and then resuspended in 0.1 M Tris-HCl, pH 7.5 or in PBS. In some embodiments, samples are stored at −20° C.

In certain aspects, pmEVs are obtained by methods adapted from Sandrini et al, 2014. In some embodiments, bacterial cultures are centrifuged at 10,000-15,500×g for 10-15 min at room temp or at 4° C. In some embodiments, cell pellets are frozen at −80° C. and supernatants are discarded. In some embodiments, cell pellets are thawed on ice and resuspended in 10 mM Tris-HCl, pH 8.0, 1 mM EDTA supplemented with 0.1 mg/mL lysozyme. In some embodiments, samples are incubated with mixing at room temp or at 37° C. for 30 min. In some embodiments, samples are re-frozen at −80° C. and thawed again on ice. In some embodiments, DNase I is added to a final concentration of 1.6 mg/mL and MgCl2 to a final concentration of 100 mM. In some embodiments, samples are sonicated using a QSonica Q500 sonicator with 7 cycles of 30 sec on and 30 sec off. In some embodiments, debris and unlysed cells are pelleted by centrifugation at 10,000×g for 15 min. at 4° C. In some embodiments, supernatants are then centrifuged at 110,000×g for 15 min at 4° C. In some embodiments, pellets are resuspended in 10 mM Tris-HCl, pH 8.0, 2% Triton X-100 and incubated 30-60 min with mixing at room temperature. In some embodiments, samples are centrifuged at 110,000×g for 15 min at 4° C. In some embodiments, pellets are resuspended in PBS and stored at −20° C.

In certain aspects, a method of forming (for example, preparing) isolated bacterial pmEVs, described herein, comprises the steps of: (a) centrifuging a bacterial culture, thereby forming a first pellet and a first supernatant, wherein the first pellet comprises cells; (b) discarding the first supernatant; (c) resuspending the first pellet in a solution; (d) lysing the cells; (e) centrifuging the lysed cells, thereby forming a second pellet and a second supernatant; (f) discarding the second pellet and centrifuging the second supernatant, thereby forming a third pellet and a third supernatant; (g) discarding the third supernatant and resuspending the third pellet in a second solution, thereby forming the isolated bacterial pmEVs.

In some embodiments, the method further comprises the steps of: (h) centrifuging the solution of step (g), thereby forming a fourth pellet and a fourth supernatant; (i) discarding the fourth supernatant and resuspending the fourth pellet in a third solution. In some embodiments, the method further comprises the steps of: (j) centrifuging the solution of step (i), thereby forming a fifth pellet and a fifth supernatant: and (k) discarding the fifth supernatant and resuspending the fifth pellet in a fourth solution.

In some embodiments, the centrifugation of step (a) is at 10,000×g. In some embodiments the centrifugation of step (a) is for 10-15 minutes. In some embodiments, the centrifugation of step (a) is at 4° C. or room temperature. In some embodiments, step (b) further comprises freezing the first pellet at −80° C. . . . In some embodiments, the solution in step (c) is 100 mM Tris-HCl, pH 7.5 supplemented with 1 mg/ml DNaseI. In some embodiments, the solution in step (c) is 10 mM Tris-HCl, pH 8.0, 1 mM EDTA, supplemented with 0.1 mg/ml lysozyme. In some embodiments, step (c) further comprises incubating for 30 minutes at 37° C. or room temperature. In some embodiments, step (c) further comprises freezing the first pellet at −80° C. . . . In some embodiments, step (c) further comprises adding DNase I to a final concentration of 1.6 mg/ml. In some embodiments, step (c) further comprises adding MgCl2 to a final concentration of 100 mM. In some embodiments, the cells are lysed in step (d) via homogenization. In some embodiments, the cells are lysed in step (d) via emulsiflex C3. In some embodiments, the cells are lysed in step (d) via sonication. In some embodiments, the cells are sonicated in 7 cycles, wherein each cycle comprises 30 seconds of sonication and 30 seconds without sonication. In some embodiments, the centrifugation of step (e) is at 10,000×g. In some embodiments, the centrifugation of step (e) is for 15 minutes. In some embodiments, the centrifugation of step (e) is at 4° C. or room temperature.

In some embodiments, the centrifugation of step (f) is at 120,000×g. In some embodiments, the centrifugation of step (f) is at 110,000×g. In some embodiments, the centrifugation of step (f) is for 1 hour. In some embodiments, the centrifugation of step (f) is for 15 minutes. In some embodiments, the centrifugation of step (f) is at 4° C. or room temperature. In some embodiments, the second solution in step (g) is 100 mM sodium carbonate, pH 11. In some embodiments, the second solution in step (g) is 10 mM Tris-HCl pH 8.0, 2% triton X-100. In some embodiments, step (g) further comprises incubating the solution for 1 hour at 4° C. In some embodiments, step (g) further comprises incubating the solution for 30-60 minutes at room temperature. In some embodiments, the centrifugation of step (h) is at 120,000×g. In some embodiments, the centrifugation of step (h) is at 110,000×g. In some embodiments, the centrifugation of step (h) is for 1 hour. In some embodiments, the centrifugation of step (h) is for 15 minutes. In some embodiments, the centrifugation of step (h) is at 4° C. or room temperature. In some embodiments, the third solution in step (i) is 100 mM Tris-HCl, pH 7.5. In some embodiments, the third solution in step (i) is PBS. In some embodiments, the centrifugation of step (j) is at 120,000×g. In some embodiments, the centrifugation of step (j) is for 20 minutes. In some embodiments, the centrifugation of step (j) is at 4° C. or room temperature. In some embodiments, the fourth solution in step (k) is 100 mM Tris-HCl, pH 7.5 or PBS.

pmEVs obtained by methods provided herein may be further purified by size based column chromatography, by affinity chromatography, and by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in 35% Optiprep in PBS. In some embodiments, if filtration was used to concentrate the filtered supernatant, the concentrate is diluted using 60% Optiprep to a final concentration of 35% Optiprep. Samples are applied to a 35-60% discontinuous sucrose gradient and centrifuged at 200,000×g for 3-24 hours at 4° C.

In some embodiments, to confirm sterility and isolation of the pmEV preparations, pmEVs are serially diluted onto agar medium used for routine culture of the bacteria being tested, and incubated using routine conditions. Non-sterile preparations are passed through a 0.22 μm filter to exclude intact cells. To further increase purity, isolated pmEVs may be DNase or proteinase K treated.

In some embodiments, the sterility of the pmEV preparations can be confirmed by plating a portion of the pmEVs onto agar medium used for standard culture of the bacteria used in the generation of the pmEVs and incubating using standard conditions.

In some embodiments select pmEVs are isolated and enriched by chromatography and binding surface moieties on pmEVs. In other embodiments, select pmEVs are isolated and/or enriched by fluorescent cell sorting by methods using affinity reagents, chemical dyes, recombinant proteins or other methods known to one skilled in the art.

In some embodiments, pmEVs are analyzed, for example, as described in Jeppesen et al. Cell 177:428 (2019).

In some embodiments, pmEVs are lyophilized.

In some embodiments, pmEVs are spray dried.

In some embodiments, pmEVs are gamma irradiated (for example, at 17.5 or 25 kGy).

In some embodiments, pmEVs are UV irradiated.

In some embodiments, pmEVs are heat inactivated (for example, at 50° C. for two hours or at 90° C. for two hours).

In some embodiments, pmEVs are acid treated.

In some embodiments, pmEVs are oxygen sparged (for example, at 0.1 vvm for two hours).

The phase of growth can affect the amount or properties of bacteria. In the methods of pmEV preparation provided herein, pmEVs can be isolated, for example, from a culture, at the start of the log phase of growth, midway through the log phase, and/or once stationary phase growth has been reached.

### Solutions and Dried Forms

The disclosure provides solutions (for example, liquid mixtures) that comprise Prevotella histicola EVs (for example, Prevotella histicola EVs and/or a combination of EVs described herein). For example, in some embodiments, a solution includes Prevotella histicola EVs and an excipient that comprises a bulking agent. As another example, in some embodiments, a solution includes Prevotella histicola EVs and an excipient that comprises a bulking agent and a lyoprotectant. As another example, in some embodiments, a solution includes Prevotella histicola EVs and an excipient that comprises a lyoprotectant.

For example, in some embodiments, the bulking agent comprises mannitol, sucrose, maltodextrin, dextran, Ficoll, or PVP-K30. In some embodiments, the excipient optionally includes an additional component such as trehalose, mannitol, sucrose, sorbitol, maltodextrin, dextran, poloxamer 188, maltodextrin, PVP-K30, Ficoll, citrate, arginine, and/or hydroxypropyl-B-cyclodextrin. For example, in some embodiments, a solution contains a liquid preparation of EVs and an excipient that comprises a bulking agent, for example, an excipient from a stock of a formula provided in one of Tables A, B, C, D, K, or P. For example, in some embodiments, a solution includes a liquid preparation containing Prevotella histicola EVs (for example, obtained by isolating Prevotella histicola EVs from a bacterial culture (such as the supernatant) or a retentate) and an excipient that comprises a bulking agent, for example, a liquid preparation containing Prevotella histicola EVs is combined with an excipient stock that comprises a bulking agent, for example, an excipient stock of a formula provided in one of Tables A, B, C, D, K, or P, to prepare the solution.

A “dried form” that contains Prevotella histicola extracellular vesicles (EVs) refers to the product resulting from drying a solution that contains Prevotella histicola EVs. In some embodiments, the drying is performed by freeze drying (lyophilization) or spray drying. In some embodiments, the dried form is a powder. As used herein, a powder refers to a type of dried form and includes a lyophilized powder, but includes powders, such as spray-dried powders, obtained by methods such as spray drying.

When freeze drying (lyophilization) is performed, the resulting product is a lyophilate. In some embodiments, the dried form is a lyophilate. As used herein, a lyophilate refers to a type of dried form and includes a lyophilized powder and lyophilized cake. In some embodiments, the lyophilized cake is milled (for example, ground) to produce a lyophilized powder. Milling refers to mechanical size reduction of solids. Grinding is a type of milling, for example, that can be performed on dried forms. See, for example, Seibert et al., “MILLING OPERATIONS IN THE PHARMACEUTICAL INDUSTRY” in Chemical Engineering in the Pharmaceutical Industry: R&D to Manufacturing, Edited by David J. am Ende (2011).

The disclosure also provides dried forms, in some embodiments, such as lyophilates, that comprise Prevotella histicola EVs (for example, Prevotella histicola EVs and/or a combination of EVs described herein), and an excipient. For example, a dried form can include Prevotella histicola EVs and an excipient that comprises a bulking agent. As another example, a dried form can include Prevotella histicola EVs and an excipient that comprises a bulking agent and a lyoprotectant. As another example, a dried form can include Prevotella histicola EVs and an excipient that comprises a lyoprotectant. For example, as described herein, in some embodiments, Prevotella histicola EVs are combined with an excipient that comprises a bulking agent and/or lyoprotectant, for example, to prepare a solution. In some embodiments, the solution is dried. The resulting dried form (for example, lyophilate) contains Prevotella histicola EVs and a component(s) of the excipient, for example, a bulking agent and/or a lyoprotectant (for example, in dried form).

The disclosure also provides dried forms of Prevotella histicola EVs and an excipient. In some embodiments, the dried form is a lyophilate, for example, such as a lyophilized cake or lyophilized powder. In some embodiments, the dried form is a powder, for example, such as a spray-dried powder or lyophilized powder. For example, in some embodiments, the bulking agent comprises mannitol, sucrose, maltodextrin, dextran, Ficoll, or PVP-K30. In some embodiments, the excipient includes an additional component such as trehalose, mannitol, sucrose, sorbitol, dextran, poloxamer 188, maltodextrin, PVP-K30, Ficoll, citrate, arginine, and/or hydroxypropyl-β-cyclodextrin. For example, in some embodiments, a dried form contains Prevotella histicola EVs and an excipient, for example, that comprises a bulking agent, for example, an excipient from a stock of a formula provided in one of Tables A, B, C, D, K, or P. In some embodiments, the dried form has a moisture content below about 6% (or below about 5%) (for example, as determined by Karl Fischer titration). In some embodiments, the dried form has about 10% to about 80% (by weight) of an excipient, for example, an excipient that comprises a bulking agent. In some embodiments, the dried form has about 10% to about 80% (by weight) of an excipient, for example, an excipient from a stock of a formula provided in one of Tables A, B, C, D, K, or P. In some embodiments, the Prevotella histicola EVs comprise about 1% to about 99% of the total solids by weight of the dried form. In some embodiments, the dried form has at least about Prevotella histicola 1e10 particles per mg of the dried form (for example, as determined by particles per mg, such as by NTA). In some embodiments, the particles of the dried form have a hydrodynamic diameter (Z average, Zₐᵥₑ) of about 130 nm to about 300 nm after resuspension from the dried form (for example, resuspension in deionized water) (for example, as determined by dynamic light scattering).

In some embodiments, the solutions and/or dried form comprise Prevotella histicola EVs substantially or entirely free of whole Prevotella histicola bacteria (for example, live bacteria, killed bacteria, and/or attenuated bacteria). In some embodiments, the solutions and/or dried form comprise both Prevotella histicola EVs and Prevotella histicola whole bacteria (for example, live bacteria, killed bacteria, and/or attenuated bacteria). In some embodiments, the solutions and/or dried form comprise gamma irradiated Prevotella histicola EVs. In some embodiments, the Prevotella histicola EVs are gamma irradiated after the EVs are isolated (for example, prepared).

In some embodiments, to quantify the numbers of Prevotella histicola EVs from bacteria from bacteria) and/or bacteria present in a bacterial sample, electron microscopy (for example, EM of ultrathin frozen sections) is used to visualize the EVs and/or bacteria and count their relative numbers. Alternatively, nanoparticle tracking analysis (NTA), Coulter counting, or dynamic light scattering (DLS) or a combination of these techniques is used. NTA and the Coulter counter count particles and show their sizes. DLS gives the size distribution of particles, but not the concentration. Bacteria frequently have diameters of 1-2 μm (microns). The full range is 0.2-20 μm. Combined results from Coulter counting and NTA can reveal the numbers of bacteria and/or EVs from bacteria in a given sample. Coulter counting reveals the numbers of particles with diameters of 0.7-10 μm. For most bacterial and/or EV samples, the Coulter counter alone can reveal the number of bacteria and/or EVs in a sample. For NTA, a Nanosight instrument can be obtained from Malvern Panalytical. For example, the NS300 can visualize and measure particles in suspension in the size range 10-2000 nm. NTA allows for counting of the numbers of particles that are, for example, 50-1000 nm in diameter. DLS reveals the distribution of particles of different diameters within an approximate range of 1 nm-3 μm.

In some embodiments, Prevotella histicola EVs are characterized by analytical methods known in the art (for example, Jeppesen, et al. Cell 177:428 (2019)).

In some embodiments, the Prevotella histicola EVs are quantified based on particle count. For example, particle count of an EV preparation can be measured using NTA. For example, particle count of an EV preparation can be measured using NTA with Zetaview.

In some embodiments, the Prevotella histicola EVs are quantified based on the amount of protein, lipid, or carbohydrate. For example, in some embodiments, total protein content of an EV preparation is measured using the Bradford assay or BCA.

In some embodiments, the Prevotella histicola EVs are isolated away from one or more other bacterial components of the source bacteria. In some embodiments, the solution and/or dried form further comprises other bacterial components.

In certain embodiments, the Prevotella histicola EV liquid preparation obtained from the source bacteria may be fractionated into subpopulations based on the physical properties (for example, size, density, protein content, and/or binding affinity) of the subpopulations. One or more of the EV subpopulations (for example, as a liquid preparation) can then be incorporated into the solutions, powders, and/or lyophilates of the invention.

In certain aspects, provided herein are solutions and/or dried forms (and therapeutic compositions thereof) comprising Prevotella histicola EVs from bacteria useful for the treatment and/or prevention of disease (for example, an immune disorder (e.g., an autoimmune disease, an inflammatory disease, an allergy), a dysbiosis, or a metabolic disease), as well as methods of making and/or identifying such EVs, and methods of using such solutions and/or dried forms (and therapeutic compositions thereof) (for example, for the treatment of an immune disorder (e.g., an autoimmune disease, an inflammatory disease, an allergy), a dysbiosis, or a metabolic disease, either alone or in combination with other therapeutics). In some embodiments, the therapeutic compositions comprise both Prevotella histicola EVs, and Prevotella histicola whole bacteria (for example, live bacteria, killed bacteria, and/or attenuated bacteria). In some embodiments, the solutions and/or dried forms comprise EVs from bacteria of one or more (e.g., 1, 2, 3, 4, 5, 6, 7, 8, 9, 10 or more) taxonomic groups (e.g., class, order, family, genus, species or strain). In some embodiments, the solutions and/or dried forms comprise EVs from bacteria of one or more (e.g., 1, 2, 3, 4, 5, 6, 7, 8, 9, 10 or more) bacteria strains or species. In some embodiments, the therapeutic compositions comprise Prevotella histicola EVs in the absence of bacteria (for example, at least about 85%, at least about 90%, at least about 95%, or at least about 99% free of bacteria). In some embodiments, the therapeutic compositions comprise EVs from bacteria of one or more (e.g., 1, 2, 3, 4, 5, 6, 7, 8, 9, 10 or more) taxonomic groups (e.g., class, order, family, genus, species or strain). In some embodiments, the therapeutic compositions comprise EVs from bacteria of one or more (e.g., 1, 2, 3, 4, 5, 6, 7, 8, 9, 10 or more) bacteria strains or species.

In some embodiments, the solution and/or dried form is added to or incorporated into a food product (for example, a food or beverage) such as a health food or beverage, a food or beverage for infants, a food or beverage for pregnant women, athletes, senior citizens or other specified group, a functional food, a beverage, a food or beverage for specified health use, a dietary supplement, a probiotic, a food or beverage for patients, or an animal feed. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood: retort pouch products such as curries, food dressed with a thick starchy sauces, soups: dairy products such as milk, dairy beverages, ice creams, cheeses, and yogurts: fermented products such as fermented soybean pastes, yogurts, fermented beverages, and pickles: bean products; various confectionery products, including biscuits, cookies, and the like, candies, chewing gums, gummies, cold desserts including jellies, cream caramels, and frozen desserts; instant foods such as instant soups and instant soy-bean soups: microwavable foods; and the like. Further, the examples also include health foods and beverages prepared in the forms of powders, granules, tablets, capsules, liquids, pastes, and jellies.

In some embodiments, the solution and/or dried form is added to a food product or food supplement for animals, including humans. The animals, other than humans, are not particularly limited, and the composition can be used for various livestock, poultry, pets, experimental animals, and the like. Specific examples of the animals include pigs, cattle, horses, sheep, goats, chickens, ducks, ostriches, turkeys, dogs, cats, rabbits, hamsters, mice, rats, monkeys, and the like, but the animals are not limited thereto.

### Therapeutic Compositions

In some embodiments, a solution and/or dried form provided herein is formulated into a therapeutic composition.

In certain embodiments, provided herein are therapeutic compositions comprising a solution and/or dried form described herein. In some embodiments, the therapeutic composition comprises a solution and/or dried form provided herein and a pharmaceutically acceptable carrier. In some embodiments, the therapeutic composition comprises a pharmaceutically acceptable excipient, such as a glidant, lubricant, and/or diluent.

In certain aspects, provided herein are therapeutic compositions comprising Prevotella histicola EVs from bacteria useful for the treatment and/or prevention of disease (for example, an immune disorder (e.g., an autoimmune disease, an inflammatory disease, an allergy), a dysbiosis, or a metabolic disease), as well as methods of making and/or identifying such EVs, and methods of using such therapeutic compositions (for example, for the treatment of an immune disorder (e.g., an autoimmune disease, an inflammatory disease, an allergy), a dysbiosis, or a metabolic disease, either alone or in combination with other therapeutics). In some embodiments, the therapeutic compositions comprise both EVs and whole bacteria (for example, live bacteria, killed bacteria, attenuated bacteria). In some embodiments, the therapeutic compositions comprise EVs in the absence of bacteria (for example, at least about 85%, at least about 90%, at least about 95%, or at least about 99% free of bacteria). In some embodiments, the therapeutic compositions comprise EVs and/or bacteria from one or more (for example, 1, 2, 3, 4, 5, 6, 7, 8, 9, 10 or more) of the bacteria from a taxonomic group. In some embodiments, the therapeutic compositions comprise EVs and/or bacteria from one or more of the bacteria strains or. In some embodiments, the therapeutic compositions comprise EVs and/or bacteria from one of the bacteria from a taxonomic group. In some embodiments, the therapeutic compositions comprise EVs and/or bacteria from one of the bacteria strains or species.

In certain aspects, provided are therapeutic compositions for administration to a subject (e.g., human subject). In some embodiments, the therapeutic compositions are combined with additional active and/or inactive materials in order to produce a final product, which may be in single dosage unit or in a multi-dose format. In some embodiments, the therapeutic composition is combined with an adjuvant such as an immuno-adjuvant (e.g., a STING agonist, a TLR agonist, or a NOD agonist).

In some embodiments, the therapeutic composition comprises at least one carbohydrate.

In some embodiments, the therapeutic composition comprises at least one lipid. In some embodiments the lipid comprises at least one fatty acid selected from lauric acid (12:0), myristic acid (14:0), palmitic acid (16:0), palmitoleic acid (16:1), margaric acid (17:0), heptadecenoic acid (17:1), stearic acid (18:0), oleic acid (18:1), linoleic acid (18:2), linolenic acid (18:3), octadecatetraenoic acid (18:4), arachidic acid (20:0), eicosenoic acid (20:1), eicosadienoic acid (20:2), eicosatetraenoic acid (20:4), eicosapentaenoic acid (20:5) (EPA), docosanoic acid (22:0), docosenoic acid (22:1), docosapentaenoic acid (22:5), docosahexaenoic acid (22:6) (DHA), and tetracosanoic acid (24:0).

In some embodiments, the therapeutic composition comprises at least one supplemental mineral or mineral source. Examples of minerals include, without limitation: chloride, sodium, calcium, iron, chromium, copper, iodine, zinc, magnesium, manganese, molybdenum, phosphorus, potassium, and selenium. Suitable forms of any of the foregoing minerals include soluble mineral salts, slightly soluble mineral salts, insoluble mineral salts, chelated minerals, mineral complexes, non-reactive minerals such as carbonyl minerals, and reduced minerals, and combinations thereof.

In some embodiments, the therapeutic composition comprises at least one supplemental vitamin. The at least one vitamin can be fat-soluble or water soluble vitamins. Suitable vitamins include but are not limited to vitamin C, vitamin A, vitamin E, vitamin B12, vitamin K, riboflavin, niacin, vitamin D, vitamin B6, folic acid, pyridoxine, thiamine, pantothenic acid, and biotin. Suitable forms of any of the foregoing are salts of the vitamin, derivatives of the vitamin, compounds having the same or similar activity of the vitamin, and metabolites of the vitamin.

In some embodiments, the therapeutic composition comprises an excipient. Non-limiting examples of suitable excipients include a buffering agent, a preservative, a stabilizer, a binder, a compaction agent, a lubricant, a dispersion enhancer, a disintegration agent, a flavoring agent, a sweetener, and a coloring agent.

In some embodiments, the excipient is a buffering agent. Non-limiting examples of suitable buffering agents include sodium citrate, magnesium carbonate, magnesium bicarbonate, calcium carbonate, and calcium bicarbonate.

In some embodiments, the excipient comprises a preservative. Non-limiting examples of suitable preservatives include antioxidants, such as alpha-tocopherol and ascorbate, and antimicrobials, such as parabens, chlorobutanol, and phenol.

In some embodiments, the therapeutic composition comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the therapeutic composition comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethyleneglycol, sodium benzoate, sodium lauryl sulfate, magnesium lauryl sulfate, and light mineral oil.

In some embodiments, the therapeutic composition comprises a dispersion enhancer as an excipient. Non-limiting examples of suitable dispersants include starch, alginic acid, polyvinylpyrrolidones, guar gum, kaolin, bentonite, purified wood cellulose, sodium starch glycolate, isomorphous silicate, and microcrystalline cellulose as high HLB emulsifier surfactants.

In some embodiments, the therapeutic composition comprises a disintegrant as an excipient. In some embodiments the disintegrant is a non-effervescent disintegrant. Non-limiting examples of suitable non-effervescent disintegrants include starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth. In some embodiments the disintegrant is an effervescent disintegrant. Non-limiting examples of suitable effervescent disintegrants include sodium bicarbonate in combination with citric acid, and sodium bicarbonate in combination with tartaric acid.

In some embodiments, the therapeutic composition is a food product (e.g., a food or beverage) such as a health food or beverage, a food or beverage for infants, a food or beverage for pregnant women, athletes, senior citizens or other specified group, a functional food, a beverage, a food or beverage for specified health use, a dietary supplement, a food or beverage for patients, or an animal feed. Specific examples of the foods and beverages include various beverages such as juices, refreshing beverages, tea beverages, drink preparations, jelly beverages, and functional beverages: alcoholic beverages such as beers: carbohydrate-containing foods such as rice food products, noodles, breads, and pastas: paste products such as fish hams, sausages, paste products of seafood: retort pouch products such as curries, food dressed with a thick starchy sauces, and Chinese soups: soups: dairy products such as milk, dairy beverages, ice creams, cheeses, and yogurts: fermented products such as fermented soy bean pastes, yogurts, fermented beverages, and pickles: bean products: various confectionery products, including biscuits, cookies, and the like, candies, chewing gums, gummies, cold desserts including jellies, cream caramels, and frozen desserts: instant foods such as instant soups and instant soy-bean soups: microwavable foods; and the like. Further, the examples also include health foods and beverages prepared in the forms of powders, granules, tablets, capsules, liquids, pastes, and jellies.

In some embodiments, the therapeutic composition is a food product for animals, including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

### Dose Forms

In some embodiments, a therapeutic composition comprising a dried form is formulated as a solid dosage form (also referred to as “solid dose form”), for example, for oral administration. In some embodiments, the solid dosage form comprises one or more excipients, for example, pharmaceutically acceptable excipients, in addition to the dried form. The dried form in the solid dosage form contains isolated Prevotella histicola EVs. Optionally, the Prevotella histicola EVs in the solid dosage form are gamma irradiated. In some embodiments, the solid dosage form comprises a tablet, a minitablet, a capsule, or a powder; or a combination of these forms (for example, minitablets comprised in a capsule).

The solid dosage form described herein can be, e.g., a capsule. The solid dosage form described herein can be, e.g., a tablet or a minitablet. Further, a plurality of minitablets can be in (e.g., loaded into) a capsule.

In certain embodiments, the solid dosage form comprises a capsule. In some embodiments, the capsule is a size 00, size 0, size 1, size 2, size 3, size 4, or size 5 capsule. In some embodiments, the capsule is a size 0 capsule. As used herein, the size of the capsule refers to the size of the tablet prior to application of an enteric coating. In some embodiments, the capsule is banded after loading (and prior to enterically coating the capsule). In some embodiments, the capsule is banded with an HPMC-based banding solution.

In some embodiments, the solid dosage form comprises a tablet (>4 mm) (e.g., 5 mm-17 mm). For example, the tablet is a 5 mm, 6 mm, 7 mm, 8 mm, 9 mm, 10 mm, 11 mm, 12 mm, 13 mm, 14 mm, 15 mm, 16 mm, 17 mm, or 18 mm tablet. The size refers to the diameter of the tablet, as is known in the art. As used herein, the size of the tablet refers to the size of the tablet prior to application of an enteric coating.

In some embodiments, the solid dosage form comprises a minitablet. The minitablet can be in the size range of 1 mm-4 mm range. E.g., the minitablet can be a 1 mm minitablet, 1.5 mm minitablet, 2 mm minitablet, 3 mm minitablet, or 4 mm minitablet. The size refers to the diameter of the minitablet, as is known in the art. As used herein, the size of the minitablet refers to the size of the minitablet prior to application of an enteric coating.

The minitablets can be in a capsule. The capsule can be a size 00, size 0, size 1, size 2, size 3, size 4, or size 5 capsule. The capsule that contains the minitablets can comprise hydroxyl propyl methyl cellulose (HPMC) or gelatin. The minitablets can be inside a capsule: the number of minitablets inside a capsule will depend on the size of the capsule and the size of the minitablets. As an example, a size 0 capsule can contain 31-35 (an average of 33) minitablets that are 3 mm minitablets. In some embodiments, the capsule is banded after loading. In some embodiments, the capsule is banded with an HPMC-based banding solution.

A therapeutic composition comprising a solution and/or powder (e.g., that comprises EVs and a bulking agent) can be formulated as a suspension (e.g., a powder can be reconstituted; a solution can be diluted), e.g., for oral administration or for injection. Administration by injection includes intravenous (IV), intramuscular (IM), and subcutaneous (SC) administration. For a suspension, EVs can be in a buffer, e.g., a pharmaceutically acceptable buffer, e.g., saline or PBS. The suspension can comprise one or more excipients, e.g., pharmaceutically acceptable excipients. The suspension can comprise, e.g., sucrose or glucose. The EVs in the solution or powder (e.g., that comprises EVs and a bulking agent) can be isolated EVs. Optionally, the EVs in the suspension can be gamma irradiated.

### Coating

A solid dosage form (e.g., capsule, tablet or minitablet) described herein can be enterically coated, e.g., with one enteric coating layer or with two layers of enteric coating, e.g., an inner enteric coating and an outer enteric coating. The inner enteric coating and outer enteric coating are not identical (e.g., the inner enteric coating and outer enteric coating do not contain the same components in the same amounts). The enteric coating allows for release of the therapeutic agent (such as Prevotella histicola EVs, dried forms, and/or solid dosage forms thereof), e.g., in the small intestine.

Release of the therapeutic agent in the small intestine allows the therapeutic agent to target and affect cells (e.g., epithelial cells and/or immune cells) located at these specific locations, e.g., which can cause a local effect in the gastrointestinal tract and/or cause a systemic effect (e.g., an effect outside of the gastrointestinal tract).

EUDRAGIT is the brand name for a diverse range of polymethacrylate-based copolymers. It includes anionic, cationic, and neutral copolymers based on methacrylic acid and methacrylic/acrylic esters or their derivatives.

Examples of other materials that can be used in the enteric coating (e.g., the one enteric coating or the inner enteric coating and/or the outer enteric coating) include cellulose acetate phthalate (CAP), cellulose acetate trimellitate (CAT), poly(vinyl acetate phthalate) (PVAP), hydroxypropyl methylcellulose phthalate (HPMCP), fatty acids, waxes, shellac (esters of aleuritic acid), plastics, plant fibers, zein, Aqua-Zein R (an aqueous zein formulation containing no alcohol), amylose starch, starch derivatives, dextrins, methyl acrylate-methacrylic acid copolymers, cellulose acetate succinate, hydroxypropyl methyl cellulose acetate succinate (hypromellose acetate succinate), methyl methacrylate-methacrylic acid copolymers, and/or sodium alginate.

The enteric coating (e.g., the one enteric coating or the inner enteric coating and/or the outer enteric coating) can include a methacrylic acid ethyl acrylate (MAE) copolymer (1:1).

The one enteric coating can include methacrylic acid ethyl acrylate (MAE) copolymer (1:1) (such as Kollicoat MAE 100P).

The one enteric coating can include a Eudragit copolymer, e.g., a Eudragit L (e.g., Eudragit L 100-55; Eudragit L 30 D-55), a Eudragit S, a Eudragit RL, a Eudragit RS, a Eudragit E, or a Eudragit FS (e.g., Eudragit FS 30 D).

Other examples of materials that can be used in the enteric coating (e.g., the one enteric coating or the inner enteric coating and/or the outer enteric coating) include those described in, e.g., U.S. Pat. Nos. 6,312,728; 6,623,759; 4,775,536; 5,047,258; 5,292,522; 6,555,124; 6,638,534; U.S. 2006/0210631; U.S. 2008/200482; U.S. 2005/0271778; U.S. 2004/0028737; WO 2005/044240.

See also, e.g., U.S. Pat. No. 9,233,074, which provides pH dependent, enteric polymers that can be used with the solid dosage forms provided herein, including methacrylic acid copolymers, polyvinylacetate phthalate, hydroxypropylmethyl cellulose acetate succinate, hydroxypropylmethyl cellulose phthalate and cellulose acetate phthalate: suitable methacrylic acid copolymers include: poly(methacrylic acid, methyl methacrylate) 1:1 sold, for example, under the Eudragit L100 trade name; poly(methacrylic acid, ethyl acrylate) 1:1 sold, for example, under the Eudragit L100-55 trade name: partially-neutralized poly(methacrylic acid, ethyl acrylate) 1:1 sold, for example, under the Kollicoat MAE-100P trade name; and poly(methacrylic acid, methyl methacrylate) 1:2 sold, for example, under the Eudragit S100 trade name.

The solid dose form (e.g., a capsule) can comprise a single layer coating, e.g., a non-enteric coating, such as HPMC (hydroxyl propyl methyl cellulose) or gelatin.

### Method of Making Solutions and Dried Forms

The disclosure also provides methods of preparing solutions of Prevotella histicola EVs and an excipient that comprises a bulking agent. For example, in some embodiments, the bulking agent comprises mannitol, sucrose, polyethylene glycol (PEG, such as PEG 6000), cyclodextrin, maltodextrin, dextran, Ficoll, or PVP-K30. In some embodiments, the excipient comprises a lyoprotectant. In some embodiments, the excipient optionally includes an additional component such as trehalose, mannitol, sucrose, sorbitol, dextran, poloxamer 188, maltodextrin, PVP-K30, Ficoll, citrate, arginine, and/or hydroxypropyl-β-cyclodextrin. For example, in some embodiments, a liquid preparation of Prevotella histicola EVs and an excipient that comprises a bulking agent are combined to prepare a solution. For example, in some embodiments, a liquid preparation of Prevotella histicola EVs (for example, obtained by isolating EVs from a bacterial culture (such as a supernatant or a retentate) and an excipient that comprises a bulking agent, for example, an excipient stock of a formula provided in one of Tables A, B, C, D, K, or P, are combined to prepare a solution. For example, in some embodiments, a liquid preparation containing Prevotella histicola EVs (for example, obtained by isolating EVs from a bacterial culture (such as a supernatant or a retentate) and an excipient that comprises a bulking agent are combined, for example, a liquid preparation containing Prevotella histicola EVs (for example, obtained by isolating EVs from a bacterial culture (such as a supernatant or a retentate) or a retentate) are combined with an excipient that comprises a bulking agent, for example, such as mannitol or an excipient of an excipient stock of a formula provided in one of Tables A, B, C, D, K, or P, to prepare the solution.

The disclosure also provides methods of preparing dried forms of Prevotella histicola EVs. For example, in some embodiments, the method is used to prepare a lyophilate such as a lyophilized powder and/or a lyophilized cake. For example, in some embodiments, the method is used to prepare a powder such as a lyophilized powder and/or a spray-dried powder. In some embodiments, the excipient comprises a bulking agent. For example, in some embodiments, the bulking agent comprises mannitol, sucrose, polyethylene glycol (PEG, such as PEG 6000), cyclodextrin, maltodextrin, dextran, Ficoll, or PVP-K30. In some embodiments, the excipient comprises a lyoprotectant. In some embodiments, the excipient optionally includes an additional component such as trehalose, mannitol, sucrose, sorbitol, dextran, poloxamer 188, maltodextrin, PVP-K30, Ficoll, citrate, arginine, and/or hydroxypropyl-B-cyclodextrin. For example, in some embodiments, a liquid preparation containing Prevotella histicola EVs (for example, obtained by isolating EVs from a bacterial culture (such as a supernatant or a retentate) is be combined with an excipient that comprises a bulking agent, such as mannitol or an excipient of an excipient stock of a formula provided in one of Tables A, B, C, D, K, or P; and dried (for example, by lyophilization or spray drying) to thereby prepare a dried form. In some embodiments, the dried form has a moisture content below about 6%, below about 5%, below about 4%, between about 0.5% to about 5%, between about 1% to about 5%, between about 1% to about 4%, between about 1.5% to about 4%, between about 2% and about 4%, or between about 2% to about 3%, (for example, as determined by Karl Fischer titration). In some embodiments, the dried form has about 10% to about 80% (by weight) of an excipient, for example, an excipient that comprises a bulking agent. In some embodiments, the dried form has about 10% to about 80% (by weight) of an excipient, for example, an excipient from a stock of a formula provided in one of Tables A, B, C, D, K, or P. In some embodiments, the Prevotella histicola EVs comprise about 1% to about 99% of the total solids by weight of the dried form. In some embodiments, the dried form has at least about 1e10 Prevotella histicola particles per mg of the dried form (for example, as determined by particles per mg, such as by NTA). In some embodiments, the particles in the dried form have a hydrodynamic diameter (Z average, Zₐᵥₑ) of about 130 nm to about 300 nm after resuspension from the dried form (for example, resuspension in deionized water) (for example, as determined by dynamic light scattering).

In some embodiments, the dried form is a lyophilate. In some embodiments, the lyophilate is a lyophilized powder or a lyophilized cake. In some embodiments, the dried form is a powder. In some embodiments, the powder is a lyophilized powder or a spray-dried powder.

In some embodiments, a method of preparing a solution that comprises extracellular vesicles (EVs) from Prevotella histicola bacteria includes: combining a liquid preparation that comprises EVs from Prevotella histicola bacteria with an excipient that comprises a bulking agent, thereby preparing the solution.

In some embodiments, a method of preparing a solution that comprises extracellular vesicles (EVs) from Prevotella histicola bacteria includes:
combining a liquid preparation that comprises EVs from Prevotella histicola bacteria with an excipient that comprises a bulking agent and a lyoprotectant, thereby preparing the solution.

In some embodiments, a method of preparing a solution that comprises extracellular vesicles (EVs) from Prevotella histicola bacteria includes: combining a liquid preparation that comprises EVs from Prevotella histicola bacteria with an excipient that comprises a lyoprotectant, thereby preparing the solution.

In some embodiments, a method of preparing a solution that comprises extracellular vesicles (EVs) from Prevotella histicola bacteria includes:
combining a liquid preparation that comprises EVs from Prevotella histicola bacteria with a stock comprising one or more excipients, wherein the stock comprises a formula provided in Table A, B, C, D, K, or P, thereby preparing a solution.

In some embodiments, the disclosure provides a solution prepared by a method described herein.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
drying the solution to prepare a cake, and milling (for example, grinding the cake, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
drying the solution to prepare a cake, and milling (for example, grinding) the cake, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
drying the solution to prepare a cake, and milling (for example, grinding) the cake, thereby preparing the dried form.

In some embodiments, the drying comprises lyophilization.

In some embodiments, the drying comprises spray drying.

In some embodiments, the method further comprises combining the dried form with an additional ingredient. In some embodiments, the additional ingredient comprises an excipient, for example, a glidant, lubricant, and/or diluent.

In some embodiments, the disclosure provides a dried form prepared by a method described herein.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
drying the solution to prepare a cake, and milling (for example, grinding) the cake, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
drying the solution to prepare a cake, and milling (for example, grinding) the cake, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
drying the solution to prepare a cake, and milling (for example, grinding) the cake, thereby preparing the powder.

In some embodiments, the drying comprises lyophilization.

In some embodiments, the drying comprises spray drying.

In some embodiments, the method further comprises combining the powder with an additional ingredient. In some embodiments, the additional ingredient comprises an excipient, for example, a glidant, lubricant, and/or diluent.

In some embodiments, the disclosure provides a powder prepared by a method described herein.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
spray drying the solution, thereby preparing the spray-dried powder.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
spray drying the solution, thereby preparing the spray-dried powder.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
spray drying the solution, thereby preparing the spray-dried powder.

In some embodiments, the method further comprises combining the spray-dried powder with an additional ingredient. In some embodiments, the additional ingredient comprises an excipient, for example, a glidant, lubricant, and/or diluent.

In some embodiments, the disclosure provides a spray-dried powder prepared by a method described herein.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
freeze drying (lyophilizing) the solution to prepare a cake, and milling (for example, grinding) the cake, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
freeze drying (lyophilizing) the solution to prepare a cake, and milling (for example, grinding) the cake, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
freeze drying (lyophilizing) the solution to prepare a cake, and milling (for example, grinding) the cake, thereby preparing the lyophilate.

In some embodiments, the method further comprises combining the lyophilate with an additional ingredient. In some embodiments, the additional ingredient comprises an excipient, for example, a glidant, lubricant, and/or diluent.

In some embodiments, the disclosure provides a lyophilate prepared by a method described herein.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution;
freeze drying (lyophilizing) the solution to prepare a cake, and
milling (for example, grinding) the cake, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution;
freeze drying (lyophilizing) the solution to prepare a cake, and milling (for example, grinding) the cake, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution;
freeze drying (lyophilizing) the solution to prepare a cake, and milling (for example, grinding) the cake, thereby preparing the lyophilized powder.

In some embodiments, the method further comprises combining the lyophilized powder with an additional ingredient. In some embodiments, the additional ingredient comprises an excipient, for example, a glidant, lubricant, and/or diluent.

In some embodiments, the disclosure provides a lyophilized powder prepared by a method described herein.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent to prepare a solution; and
freeze drying (lyophilizing) the solution, thereby preparing the lyophilized cake.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a bulking agent and a lyoprotectant to prepare a solution; and
freeze drying (lyophilizing) the solution, thereby preparing the lyophilized cake.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with an excipient that comprises (or consists essentially of) a lyoprotectant to prepare a solution; and
freeze drying (lyophilizing) the solution, thereby preparing the lyophilized cake.

In some embodiments, the disclosure provides a lyophilized cake prepared by a method described herein.

In some aspects, the disclosure provides a method of preparing a solution that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution.

In some embodiments, the disclosure provides a solution prepared by a method described herein.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
drying the solution, thereby preparing the dried form.

In some aspects, the disclosure provides a method of preparing a dried form that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; drying the solution to prepare a cake, and
milling (for example, grinding) the cake, thereby preparing the dried form.

In some embodiments, the drying comprises lyophilization.

In some embodiments, the drying comprises spray drying.

In some embodiments, the method further comprises combining the dried form with an additional ingredient. In some embodiments, the additional ingredient comprises an excipient, for example, a glidant, lubricant, and/or diluent.

In some embodiments, the disclosure provides a dried form prepared by a method described herein.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
drying the solution, thereby preparing the powder.

In some aspects, the disclosure provides a method of preparing a powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
drying the solution to prepare a cake, and
milling (for example, grinding) the cake, thereby preparing the powder.

In some embodiments, the drying comprises lyophilization.

In some embodiments, the drying comprises spray drying.

In some embodiments, the method further comprises combining the powder with an additional ingredient. In some embodiments, the additional ingredient comprises an excipient, for example, a glidant, lubricant, and/or diluent.

In some embodiments, the disclosure provides a powder prepared by a method described herein.

In some aspects, the disclosure provides a method of preparing a spray-dried powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
spray drying the solution, thereby preparing the spray-dried powder.

In some embodiments, the method further comprises combining the spray-dried powder with an additional ingredient. In some embodiments, the additional ingredient comprises an excipient, for example, a glidant, lubricant, and/or diluent.

In some embodiments, the disclosure provides a spray-dried powder prepared by a method described herein.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing the lyophilate.

In some aspects, the disclosure provides a method of preparing a lyophilate that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
freeze drying (lyophilizing) the solution to prepare a cake, and
milling (for example, grinding) the cake, thereby preparing the lyophilate.

In some embodiments, the method further comprises combining the lyophilate with an additional ingredient. In some embodiments, the additional ingredient comprises an excipient, for example, a glidant, lubricant, and/or diluent.

In some embodiments, the disclosure provides a lyophilate prepared by a method described herein.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing the lyophilized powder.

In some aspects, the disclosure provides a method of preparing a lyophilized powder that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution;
freeze drying (lyophilizing) the solution to prepare a cake, and
milling (for example, grinding) the cake, thereby preparing the lyophilized powder.

In some embodiments, the method further comprises combining the lyophilized powder with an additional ingredient. In some embodiments, the additional ingredient comprises an excipient, for example, a glidant, lubricant, and/or diluent.

In some embodiments, the disclosure provides a lyophilized powder prepared by a method described herein.

In some aspects, the disclosure provides a method of preparing a lyophilized cake that comprises extracellular vesicles (EVs) from Prevotella histicola bacteria, the method comprising:
combining a liquid preparation that comprises EVs from Prevotella histicola bacteria with a stock comprising one or more excipients, wherein the stock comprises a formula provided in Table A, B, C, D, K, or P, thereby preparing a solution; and
freeze drying (lyophilizing) the solution, thereby preparing a lyophilized cake.

In some embodiments, the disclosure provides a lyophilized cake prepared by a method described herein.

### Method of Preparing Therapeutic Compositions

The disclosure also provides methods of preparing therapeutic compositions. In some embodiments, the method includes combining a solution or dried form described herein with a pharmaceutically acceptable excipient, such as a glidant, lubricant, and/or diluent, thereby preparing a therapeutic composition.

The disclosure also provides methods of preparing therapeutic compositions, such as solid dosage forms, that contain a dried form described herein. In some embodiments, the solid dosage form is a capsule, tablet, or minitablet.

The disclosure also provides methods of making a solid dosage form (for example, for oral administration) (for example, for pharmaceutical use) that comprises a dried form. In some embodiments, the dried form comprises Prevotella histicola extracellular vesicles (EVs) and an excipient that comprises a bulking agent. In some embodiments, the dried form comprises Prevotella histicola extracellular vesicles (EVs) and an excipient that comprises a lyoprotectant. In some embodiments, the dried form comprises Prevotella histicola extracellular vesicles (EVs) and an excipient that comprises a bulking agent and a lyoprotectant. In some embodiments, the dried form also contains one or more additional components. In some embodiments, the dried form is combined with one or more pharmaceutically acceptable excipients. In some embodiments, the solid dosage form is enterically coated, for example, with a coating described herein.

In some aspects, a method of making the solid dosage form includes: loading a dried form that comprises Prevotella histicola extracellular vesicles (EVs) into a capsule, thereby preparing a capsule, and thereby preparing the solid dosage form:
optionally combining the dried form with a pharmaceutically acceptable excipient prior to loading into the capsule; and/or
optionally banding the capsule after loading the capsule (for example, optionally banding the capsule after loading the capsule).

In some aspects, a method of making the solid dosage form includes:
compressing a dried form that comprises Prevotella histicola extracellular vesicles (EVs) described herein into a minitablet, thereby preparing a minitablet and thereby preparing the solid dosage form;
optionally combining the dried form with a pharmaceutically acceptable excipient prior to compressing;
optionally filling a capsule with a plurality of enterically coated minitablets.

In some aspects, a method of making the solid dosage form includes:
compressing a dried form that comprises Prevotella histicola extracellular vesicles (EVs) described herein into a tablet, thereby preparing a tablet, and thereby preparing the solid dosage form;
optionally combining the dried form with a pharmaceutically acceptable excipient prior to compressing.

In certain embodiments, the method comprises performing wet granulation on a powder prior to combining the powder and one or more (for example, one, two or three) excipients into a therapeutic composition, such as a solid dosage form. In some embodiments, the wet granulation comprises (i) mixing the powder with a granulating fluid (for example, water, ethanol, or isopropanol, alone or in combination). In some embodiments, the wet granulation comprises mixing the powder with water. In some embodiments, the wet granulation comprises (ii) drying mixed powder and granulating fluid (for example, drying on a fluid bed dryer). In some embodiments, the wet granulation comprises (iii) milling (for example, grinding) the dried powder and granulating fluid. The milled (for example, ground) powder and granulating fluid are then combined with the one or more (for example, one, two or three) excipients to prepare a therapeutic composition, such as a solid dosage form. In some embodiments, the powder is a lyophilized powder. In some embodiments, the powder is a spray-dried powder.

In some embodiments, a dried form described herein is reconstituted in a liquid (for example, a buffer, juice, or water) to prepare a therapeutic composition.

In some embodiments, a solution is resuspended (for example, diluted) in a liquid (for example, a buffer, juice, or water) to prepare a therapeutic composition.

In some embodiments, a therapeutic composition comprising a dried form described herein is reconstituted in a liquid (for example, a buffer, juice, or water) to prepare a suspension.

In some embodiments, a therapeutic composition comprising a solution is resuspended (for example, diluted) in a liquid (for example, a buffer, juice, or water) to prepare a suspension.

### Gamma-Irradiation

Powders (e.g., of EVs from Prevotella histicola bacteria) can be gamma-irradiated at 17.5 kGy radiation unit at ambient temperature.

Frozen biomasses (e.g., of EVs from Prevotella histicola bacteria) can be gamma-irradiated at 25 kGy radiation unit in the presence of dry ice.

### Additional Therapeutic Agents

In certain aspects, the methods provided herein include the administration to a subject of a therapeutic composition described herein either alone or in combination with an additional therapeutic agent. In some embodiments, the additional therapeutic agent is an immunosuppressant, an anti-inflammatory agent, and/or a steroid.

In some embodiments, the therapeutic composition comprising EVs from Prevotella histicola bacteria is administered to the subject before the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, the therapeutic composition comprising EVs from Prevotella histicola bacteria is administered to the subject after the additional therapeutic agent is administered (e.g., at least 1, 2, 3, 4, 5, 6, 7, 8, 9, 10, 11, 12, 13, 14, 15, 16, 17, 18, 19, 20, 21, 22, 23 or 24 hours after or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the therapeutic composition comprising EVs from Prevotella histicola bacteria and the additional therapeutic agent are administered to the subject simultaneously or nearly simultaneously (e.g., administrations occur within an hour of each other).

In some embodiments, an antibiotic is administered to the subject before the therapeutic composition comprising EVs from Prevotella histicola bacteria is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days before). In some embodiments, an antibiotic is administered to the subject after therapeutic composition comprising EVs from Prevotella histicola bacteria is administered to the subject (e.g., at least 1, 2, 3, 4, 5, 6, 7, 8, 9, 10, 11, 12, 13, 14, 15, 16, 17, 18, 19, 20, 21, 22, 23 or 24 hours before or at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days after). In some embodiments, the therapeutic composition comprising EVs from Prevotella histicola bacteria and the antibiotic are administered to the subject simultaneously or nearly simultaneously (e.g., administrations occur within an hour of each other).

In some embodiments, the methods provided herein include the administration of a therapeutic composition described herein in combination with one or more additional therapeutic agents. In some embodiments, the methods disclosed herein include the administration of two therapeutic agents.

In some embodiments, the therapeutic agent is an antibiotic. “Antibiotics” broadly refers to compounds capable of inhibiting or preventing a bacterial infection. Antibiotics can be classified in a number of ways, including their use for specific infections, their mechanism of action, their bioavailability, or their spectrum of target microbe (e.g., Gram-negative vs. Gram-positive bacteria, aerobic vs. anaerobic bacteria, etc.) and these may be used to kill specific bacteria in specific areas of the host (“niches”) (Leekha, et al 2011. General Principles of Antimicrobial Therapy. Mayo Clin Proc. 86 (2): 156-167). In certain embodiments, antibiotics can be used to selectively target bacteria of a specific niche. In some embodiments, antibiotics are administered after the therapeutic composition comprising EVs from Prevotella histicola bacteria. In some embodiments, antibiotics are administered before therapeutic composition comprising EVs from Prevotella histicola bacteria.

In some aspects, antibiotics can be selected based on their bactericidal or bacteriostatic properties. Bactericidal antibiotics include mechanisms of action that disrupt the cell wall (e.g., β-lactams), the cell membrane (e.g., daptomycin), or bacterial DNA (e.g., fluoroquinolones). Bacteriostatic agents inhibit bacterial replication and include sulfonamides, tetracyclines, and macrolides, and act by inhibiting protein synthesis. Furthermore, while some drugs can be bactericidal in certain organisms and bacteriostatic in others, knowing the target organism allows one skilled in the art to select an antibiotic with the appropriate properties. In certain treatment conditions, bacteriostatic antibiotics inhibit the activity of bactericidal antibiotics. Thus, in certain embodiments, bactericidal and bacteriostatic antibiotics are not combined.

Antibiotics include, but are not limited to aminoglycosides, ansamycins, carbacephems, carbapenems, cephalosporins, glycopeptides, lincosamides, lipopeptides, macrolides, monobactams, nitrofurans, oxazolidonones, penicillins, polypeptide antibiotics, quinolones, fluoroquinolone, sulfonamides, tetracyclines, and anti-mycobacterial compounds, and combinations thereof.

Aminoglycosides include, but are not limited to Amikacin, Gentamicin, Kanamycin, Neomycin, Netilmicin, Tobramycin, Paromomycin, and Spectinomycin. Aminoglycosides are effective, e.g., against Gram-negative bacteria, such as Escherichia coli, Klebsiella, Pseudomonas aeruginosa, and Francisella tularensis, and against certain aerobic bacteria but less effective against obligate/facultative anaerobes. Aminoglycosides are believed to bind to the bacterial 30S or 50S ribosomal subunit thereby inhibiting bacterial protein synthesis.

Ansamycins include, but are not limited to, Geldanamycin, Herbimycin, Rifamycin, and Streptovaricin. Geldanamycin and Herbimycin are believed to inhibit or alter the function of Heat Shock Protein 90.

Carbacephems include, but are not limited to, Loracarbef. Carbacephems are believed to inhibit bacterial cell wall synthesis.

Carbapenems include, but are not limited to, Ertapenem, Doripenem, Imipenem/Cilastatin, and Meropenem. Carbapenems are bactericidal for both Gram-positive and Gram-negative bacteria as broad-spectrum antibiotics. Carbapenems are believed to inhibit bacterial cell wall synthesis.

Cephalosporins include, but are not limited to, Cefadroxil, Cefazolin, Cefalotin, Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil, and Ceftobiprole. Selected Cephalosporins are effective, e.g., against Gram-negative bacteria and against Gram-positive bacteria, including Pseudomonas, certain Cephalosporins are effective against methicillin-resistant Staphylococcus aureus (MRSA). Cephalosporins are believed to inhibit bacterial cell wall synthesis by disrupting synthesis of the peptidoglycan layer of bacterial cell walls.

Glycopeptides include, but are not limited to, Teicoplanin, Vancomycin, and Telavancin. Glycopeptides are effective, e.g., against aerobic and anaerobic Gram-positive bacteria including MRSA and Clostridium difficile. Glycopeptides are believed to inhibit bacterial cell wall synthesis by disrupting synthesis of the peptidoglycan layer of bacterial cell walls.

Lincosamides include, but are not limited to, Clindamycin and Lincomycin. Lincosamides are effective, e.g., against anaerobic bacteria, as well as Staphylococcus, and Streptococcus. Lincosamides are believed to bind to the bacterial 50S ribosomal subunit thereby inhibiting bacterial protein synthesis.

Lipopeptides include, but are not limited to, Daptomycin. Lipopeptides are effective, e.g., against Gram-positive bacteria. Lipopeptides are believed to bind to the bacterial membrane and cause rapid depolarization.

Macrolides include, but are not limited to, Azithromycin, Clarithromycin, Dirithromycin, Erythromycin, Roxithromycin, Troleandomycin, Telithromycin, and Spiramycin. Macrolides are effective, e.g., against Streptococcus and Mycoplasma. Macrolides are believed to bind to the bacterial or 50S ribosomal subunit, thereby inhibiting bacterial protein synthesis.

Monobactams include, but are not limited to, Aztreonam. Monobactams are effective, e.g., against Gram-negative bacteria. Monobactams are believed to inhibit bacterial cell wall synthesis by disrupting synthesis of the peptidoglycan layer of bacterial cell walls.

Nitrofurans include, but are not limited to, Furazolidone and Nitrofurantoin.

Oxazolidonones include, but are not limited to, Linezolid, Posizolid, Radezolid, and Torezolid. Oxazolidonones are believed to be protein synthesis inhibitors.

Penicillins include, but are not limited to, Amoxicillin, Ampicillin, Azlocillin, Carbenicillin, Cloxacillin, Dicloxacillin, Flucloxacilline, Mezlocillin, Methicillin, Nafcillin, Oxacillin, Penicillin G, Penicillin V, Piperacillin, Temocillin and Ticarcillin. Penicillins are effective, e.g., against Gram-positive bacteria, facultative anaerobes, e.g., Streptococcus, Borrelia, and Treponema. Penicillins are believed to inhibit bacterial cell wall synthesis by disrupting synthesis of the peptidoglycan layer of bacterial cell walls.

Penicillin combinations include, but are not limited to, Amoxicillin/clavulanate, Ampicillin/sulbactam, Piperacillin/tazobactam, and Ticarcillin/clavulanate.

Polypeptide antibiotics include, but are not limited to, Bacitracin, Colistin, and Polymyxin B and E. Polypeptide Antibiotics are effective, e.g., against Gram-negative bacteria. Certain polypeptide antibiotics are believed to inhibit isoprenyl pyrophosphate involved in synthesis of the peptidoglycan layer of bacterial cell walls, while others destabilize the bacterial outer membrane by displacing bacterial counter-ions.

Quinolones and Fluoroquinolone include, but are not limited to, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, and Temafloxacin. Quinolones/Fluoroquinolone are effective, e.g., against Streptococcus and Neisseria. Quinolones/Fluoroquinolone are believed to inhibit the bacterial DNA gyrase or topoisomerase IV, thereby inhibiting DNA replication and transcription.

Sulfonamides include, but are not limited to, Mafenide, Sulfacetamide, Sulfadiazine, Silver sulfadiazine, Sulfadimethoxine, Sulfamethizole, Sulfamethoxazole, Sulfanilamide, Sulfasalazine, Sulfisoxazole, Trimethoprim-Sulfamethoxazole (Co-trimoxazole), and Sulfonamidochrysoidine. Sulfonamides are believed to inhibit folate synthesis by competitive inhibition of dihydropteroate synthetase, thereby inhibiting nucleic acid synthesis.

Tetracyclines include, but are not limited to, Demeclocycline, Doxycycline, Minocycline, Oxytetracycline, and Tetracycline. Tetracyclines are effective, e.g., against Gram-negative bacteria. Tetracyclines are believed to bind to the bacterial 30S ribosomal subunit thereby inhibiting bacterial protein synthesis.

Anti-mycobacterial compounds include, but are not limited to, Clofazimine, Dapsone, Capreomycin, Cycloserine, Ethambutol, Ethionamide, Isoniazid, Pyrazinamide, Rifampicin, Rifabutin, Rifapentine, and Streptomycin.

Suitable antibiotics also include arsphenamine, chloramphenicol, fosfomycin, fusidic acid, metronidazole, mupirocin, platensimycin, quinupristin/dalfopristin, tigecycline, tinidazole, trimethoprim amoxicillin/clavulanate, ampicillin/sulbactam, amphomycin ristocetin, azithromycin, bacitracin, buforin II, carbomycin, cecropin Pl, clarithromycin, erythromycins, furazolidone, fusidic acid, Na fusidate, gramicidin, imipenem, indolicidin, josamycin, magainin II, metronidazole, nitroimidazoles, mikamycin, mutacin B-Ny266, mutacin B-JH1 140, mutacin J-T8, nisin, nisin A, novobiocin, oleandomycin, ostreogrycin, piperacillin/tazobactam, pristinamycin, ramoplanin, ranalexin, reuterin, rifaximin, rosamicin, rosaramicin, spectinomycin, spiramycin, staphylomycin, streptogramin, streptogramin A, synergistin, taurolidine, teicoplanin, telithromycin, ticarcillin/clavulanic acid, triacetyloleandomycin, tylosin, tyrocidin, tyrothricin, vancomycin, vemamycin, and virginiamycin.

In some embodiments, the additional therapeutic agent is an immunosuppressive agent, a DMARD, a pain-control drug, a steroid, a non-steroidal anti-inflammatory drug (NSAID), or a cytokine antagonist, and combinations thereof. Representative agents include, but are not limited to, cyclosporine, retinoids, corticosteroids, propionic acid derivative, acetic acid derivative, enolic acid derivatives, fenamic acid derivatives, Cox-2 inhibitors, lumiracoxib, ibuprophen, cholin magnesium salicylate, fenoprofen, salsalate, difunisal, tolmetin, ketoprofen, flurbiprofen, oxaprozin, indomethacin, sulindac, etodolac, ketorolac, nabumetone, naproxen, valdecoxib, etoricoxib, MK0966: rofecoxib, acetominophen, Celecoxib, Diclofenac, tramadol, piroxicam, meloxicam, tenoxicam, droxicam, lornoxicam, isoxicam, mefenamic acid, meclofenamic acid, flufenamic acid, tolfenamic, valdecoxib, parecoxib, etodolac, indomethacin, aspirin, ibuprophen, firocoxib, methotrexate (MTX), antimalarial drugs (e.g., hydroxychloroquine and chloroquine), sulfasalazine, Leflunomide, azathioprine, cyclosporin, gold salts, minocycline, cyclophosphamide, D-penicillamine, minocycline, auranofin, tacrolimus, myocrisin, chlorambucil, TNF alpha antagonists (e.g., TNF alpha antagonists or TNF alpha receptor antagonists), e.g., ADALIMUMAB (Humira®), ETANERCEPT (Enbrel®), INFLIXIMAB (Remicade®; TA-650), CERTOLIZUMAB PEGOL (Cimzia®; CDP870), GOLIMUMAB (Simpom®; CNTO 148), ANAKINRA (Kineret®), RITUXIMAB (Rituxan®; MabThera®), ABATACEPT (Orencia®), TOCILIZUMAB (RoActemra/Actemra®), integrin antagonists (TYSABRI®) (natalizumab)), IL-1 antagonists (ACZ885 (Ilaris)), Anakinra (Kineret®)), CD4 antagonists, IL-23 antagonists, IL-20 antagonists, IL-6 antagonists, BLyS antagonists (e.g., Atacicept, Benlysta R/LymphoStat-BR (belimumab)), p38 Inhibitors, CD20 antagonists (Ocrelizumab, Ofatumumab (Arzerra R))), interferon gamma antagonists (Fontolizumab), prednisolone, Prednisone, dexamethasone, Cortisol, cortisone, hydrocortisone, methylprednisolone, betamethasone, triamcinolone, beclometasone, fludrocortisone, deoxycorticosterone, aldosterone, Doxycycline, vancomycin, pioglitazone, SBI-087, SCIO-469, Cura-100, Oncoxin+Viusid, TwHF, Methoxsalen, Vitamin D-ergocalciferol, Milnacipran, Paclitaxel, rosig tazone, Tacrolimus (Prograf®), RADOO1, rapamune, rapamycin, fostamatinib, Fentanyl, XOMA 052, Fostamatinib disodium, rosightazone, Curcumin (Longvida™), Rosuvastatin, Maraviroc, ramipn1, Milnacipran, Cobiprostone, somatropin, tgAAC94 gene therapy vector, MK0359, GW856553, esomeprazole, everolimus, trastuzumab, JAK1 and JAK2 inhibitors, pan JAK inhibitors, e.g., tetracyclic pyridone 6 (P6), 325, PF-956980, denosumab, IL-6 antagonists, CD20 antagonists, CTLA4 antagonists, IL-8 antagonists, IL-21 antagonists, IL-22 antagonist, integrin antagonists (Tysarbri® (natalizumab)), VGEF antagonists, CXCL antagonists, MMP antagonists, defensin antagonists, IL-1 antagonists (including IL-1 beta antagonists), and IL-23 antagonists (e.g., receptor decoys, antagonistic antibodies, etc.).

In some embodiments, the additional therapeutic agent is an immunosuppressive agent. Examples of immunosuppressive agents include, but are not limited to, corticosteroids, mesalazine, mesalamine, sulfasalazine, sulfasalazine derivatives, immunosuppressive drugs, cyclosporin A, mercaptopurine, azathioprine, prednisone, methotrexate, antihistamines, glucocorticoids, epinephrine, theophylline, cromolyn sodium, anti-leukotrienes, anti-cholinergic drugs for rhinitis, TLR antagonists, inflammasome inhibitors, anti-cholinergic decongestants, mast-cell stabilizers, monoclonal anti-IgE antibodies, vaccines (e.g., vaccines used for vaccination where the amount of an allergen is gradually increased), cytokine inhibitors, such as anti-IL-6 antibodies, TNF inhibitors such as infliximab, adalimumab, certolizumab pegol, golimumab, or etanercept, and combinations thereof.

### Administration

In certain aspects, provided herein is a method of delivering a therapeutic composition described herein (e.g., a therapeutic composition EVs from Prevotella histicola bacteria) to a subject. In some embodiments of the methods provided herein, the therapeutic composition is administered in conjunction with the administration of an additional therapeutic agent. In some embodiments, the therapeutic composition comprises EVs from Prevotella histicola bacteria co-formulated with the additional therapeutic agent. In some embodiments, the therapeutic composition comprising EVs from Prevotella histicola bacteria is co-administered with the additional therapeutic agent. In some embodiments, the additional therapeutic agent is administered to the subject before administration of the therapeutic composition that comprises EVs from Prevotella histicola bacteria (e.g., about 1, 2, 3, 4, 5, 6, 7, 8, 9, 10, 15, 20, 25, 30, 35, 40, 45, 50 or 55 minutes before, about 1, 2, 3, 4, 5, 6, 7, 8, 9, 10, 11, 12, 13, 14, 15, 16, 17, 18, 19, 20, 21, 22 or 23 hours before, or about 1, 2, 3, 4, 5, 6, 7, 8, 9, 10, 11, 12, 13 or 14 days before). In some embodiments, the additional therapeutic agent is administered to the subject after administration of the therapeutic composition that comprises EVs from Prevotella histicola bacteria (e.g., about 1, 2, 3, 4, 5, 6, 7, 8, 9, 10, 15, 20, 25, 30, 35, 40, 45, 50 or 55 minutes after, about 1, 2, 3, 4, 5, 6, 7, 8, 9, 10, 11, 12, 13, 14, 15, 16, 17, 18, 19, 20, 21, 22 or 23 hours after, or about 1, 2, 3, 4, 5, 6, 7, 8, 9, 10, 11, 12, 13 or 14 days after). In some embodiments, the same mode of delivery is used to deliver both the therapeutic composition that comprises EVs from Prevotella histicola bacteria and the additional therapeutic agent. In some embodiments, different modes of delivery are used to administer the therapeutic composition that comprises EVs from Prevotella histicola bacteria and the additional therapeutic agent. For example, in some embodiments, the therapeutic composition that comprises EVs from Prevotella histicola bacteria is administered orally while the additional therapeutic agent is administered via injection (e.g., an intravenous and/or intramuscular injection).

In some embodiments, the therapeutic composition described herein is administered once a day. In some embodiments, the therapeutic composition described herein is administered twice a day. In some embodiments, the therapeutic composition described herein is formulated for a daily dose. In some embodiments, the therapeutic composition described herein is formulated for a twice a day dose, wherein each dose is half of the daily dose.

The dosage regimen can be any of a variety of methods and amounts, and can be determined by one skilled in the art according to known clinical factors. As is known in the medical arts, dosages for any one patient can depend on many factors, including the subject's species, size, body surface area, age, sex, immunocompetence, and general health, the particular microorganism to be administered, duration and route of administration, the kind and stage of the disease, for example, and other compounds such as drugs being administered concurrently or near-concurrently. In addition to the above factors, such levels can be affected by the infectivity of the microorganism, and the nature of the microorganism, as can be determined by one skilled in the art. In the present methods, appropriate minimum dosage levels of microorganisms can be levels sufficient for the microorganism to survive, grow and replicate. The dose of a therapeutic composition that comprises EVs from Prevotella histicola bacteria described herein may be appropriately set or adjusted in accordance with the dosage form, the route of administration, the degree or stage of a target disease, and the like. For example, the general effective dose of the agents may range between 0.01 mg/kg body weight/day and 1000 mg/kg body weight/day, between 0.1 mg/kg body weight/day and 1000 mg/kg body weight/day, 0.5 mg/kg body weight/day and 500 mg/kg body weight/day, 1 mg/kg body weight/day and 100 mg/kg body weight/day, or between 5 mg/kg body weight/day and 50 mg/kg body weight/day. The effective dose may be 0.01, 0.05, 0.1, 0.5, 1, 2, 3, 5, 10, 20, 30, 40, 50, 60, 70, 80, 90, 100, 200, 500, or 1000 mg/kg body weight/day or more, but the dose is not limited thereto.

In some embodiments, the dose administered to a subject is sufficient to prevent disease (e.g., an immune disorder (e.g., an autoimmune disease, an inflammatory disease, an allergy), a dysbiosis, or a metabolic disease), delay its onset, or slow or stop its progression, or relieve one or more symptoms of the disease. One skilled in the art will recognize that dosage will depend upon a variety of factors including the strength of the particular agent (e.g., therapeutic agent) employed, as well as the age, species, condition, and body weight of the subject. The size of the dose will also be determined by the route, timing, and frequency of administration, as well as the existence, nature, and extent of any adverse side-effects that might accompany the administration of a particular therapeutic agent and the desired physiological effect.

Suitable doses and dosage regimens can be determined by conventional range-finding techniques known to those of ordinary skill in the art. Generally, treatment is initiated with smaller dosages, which are less than the optimum dose of the compound. Thereafter, the dosage is increased by small increments until the optimum effect under the circumstances is reached. An effective dosage and treatment protocol can be determined by routine and conventional means, starting e.g., with a low dose in laboratory animals and then increasing the dosage while monitoring the effects, and systematically varying the dosage regimen as well. Animal studies are commonly used to determine the maximal tolerable dose (“MTD”) of bioactive agent per kilogram weight. Those skilled in the art regularly extrapolate doses for efficacy, while avoiding toxicity, in other species, including humans.

In accordance with the above, in therapeutic applications, the dosages of the therapeutic agents used in accordance with the invention vary depending on the active agent, the age, weight, and clinical condition of the recipient patient, and the experience and judgment of the clinician or practitioner administering the therapy, among other factors affecting the selected dosage. As another example, the dose should be sufficient to result in slowing of progression of the disease for which the subject is being treated, and preferably amelioration of one or more symptoms of the disease for which the subject is being treated.

Separate administrations can include any number of two or more administrations, including two, three, four, five or six administrations. One skilled in the art can readily determine the number of administrations to perform or the desirability of performing one or more additional administrations according to methods known in the art for monitoring therapeutic methods and other monitoring methods provided herein. Accordingly, the methods provided herein include methods of providing to the subject one or more administrations of a therapeutic composition, where the number of administrations can be determined by monitoring the subject, and, based on the results of the monitoring, determining whether or not to provide one or more additional administrations. Deciding on whether or not to provide one or more additional administrations can be based on a variety of monitoring results.

The time period between administrations can be any of a variety of time periods. The time period between administrations can be a function of any of a variety of factors, including monitoring steps, as described in relation to the number of administrations, the time period for a subject to mount an immune response. In one example, the time period can be a function of the time period for a subject to mount an immune response; for example, the time period can be more than the time period for a subject to mount an immune response, such as more than about one week, more than about ten days, more than about two weeks, or more than about a month: in another example, the time period can be less than the time period for a subject to mount an immune response, such as less than about one week, less than about ten days, less than about two weeks, or less than about a month.

In some embodiments, the delivery of an additional therapeutic agent in combination with the therapeutic composition described herein reduces the adverse effects and/or improves the efficacy of the additional therapeutic agent.

The effective dose of an additional therapeutic agent described herein is the amount of the additional therapeutic agent that is effective to achieve the desired therapeutic response for a particular subject, composition, and mode of administration, with the least toxicity to the subject. The effective dosage level can be identified using the methods described herein and will depend upon a variety of pharmacokinetic factors including the activity of the particular compositions or agents administered, the route of administration, the time of administration, the rate of excretion of the particular compound being employed, the duration of the treatment, other drugs, compounds and/or materials used in combination with the particular compositions employed, the age, sex, weight, condition, general health and prior medical history of the subject being treated, and like factors well known in the medical arts. In general, an effective dose of an additional therapeutic agent will be the amount of the additional therapeutic agent that is the lowest dose effective to produce a therapeutic effect. Such an effective dose will generally depend upon the factors described above.

The toxicity of an additional therapeutic agent is the level of adverse effects experienced by the subject during and following treatment. Adverse events associated with additional therapy toxicity can include, but are not limited to, abdominal pain, acid indigestion, acid reflux, allergic reactions, alopecia, anaphylaxis, anemia, anxiety, lack of appetite, arthralgias, asthenia, ataxia, azotemia, loss of balance, bone pain, bleeding, blood clots, low blood pressure, elevated blood pressure, difficulty breathing, bronchitis, bruising, low white blood cell count, low red blood cell count, low platelet count, cardiotoxicity, cystitis, hemorrhagic cystitis, arrhythmias, heart valve disease, cardiomyopathy, coronary artery disease, cataracts, central neurotoxicity, cognitive impairment, confusion, conjunctivitis, constipation, coughing, cramping, cystitis, deep vein thrombosis, dehydration, depression, diarrhea, dizziness, dry mouth, dry skin, dyspepsia, dyspnea, edema, electrolyte imbalance, esophagitis, fatigue, loss of fertility, fever, flatulence, flushing, gastric reflux, gastroesophageal reflux disease, genital pain, granulocytopenia, gynecomastia, glaucoma, hair loss, hand-foot syndrome, headache, hearing loss, heart failure, heart palpitations, heartburn, hematoma, hemorrhagic cystitis, hepatotoxicity, hyperamylasemia, hypercalcemia, hypercholemia, hyperglycemia, hyperkalemia, hyperlipidemia, hypomagnesemia, hypernatremia, hyperphosphatemia, hyperpigmentation, hypertriglyceridemia, hyperuricemia, hypoalbuminemia, hypocalcemia, hypochloremia, hypoglycemia, hypokalemia, hypomagnesemia, hyponatremia, hypophosphatemia, impotence, infection, injection site reactions, insomnia, iron deficiency, itching, joint pain, kidney failure, leukopenia, liver dysfunction, memory loss, menopause, mouth sores, mucositis, muscle pain, myalgias, myelosuppression, myocarditis, neutropenic fever, nausea, nephrotoxicity, neutropenia, nosebleeds, numbness, ototoxicity, pain, palmar-plantar erythrodysesthesia, pancytopenia, pericarditis, peripheral neuropathy, pharyngitis, photophobia, photosensitivity, pneumonia, pneumonitis, proteinuria, pulmonary embolus, pulmonary fibrosis, pulmonary toxicity, rash, rapid heartbeat, rectal bleeding, restlessness, rhinitis, seizures, shortness of breath, sinusitis, thrombocytopenia, tinnitus, urinary tract infection, vaginal bleeding, vaginal dryness, vertigo, water retention, weakness, weight loss, weight gain, and xerostomia. In general, toxicity is acceptable if the benefits to the subject achieved through the therapy outweigh the adverse events experienced by the subject due to the therapy.

### Immune Disorders

In some embodiments, the methods and therapeutic compositions described herein relate to the treatment or prevention of a disease or disorder associated a pathological immune response, such as an autoimmune disease, an allergic reaction and/or an inflammatory disease. In some embodiments, the disease or disorder is an inflammatory bowel disease (e.g., Crohn's disease or ulcerative colitis). In some embodiments, the disease or disorder is psoriasis. In some embodiments, the disease or disorder is atopic dermatitis.

The methods described herein can be used to treat any subject in need thereof. As used herein, a “subject in need thereof” includes any subject that has a disease or disorder associated with a pathological immune response (e.g., an inflammatory bowel disease), as well as any subject with an increased likelihood of acquiring a such a disease or disorder.

The therapeutic compositions described herein can be used, for example, as a therapeutic (such as pharmaceutical) composition for preventing or treating (reducing, partially or completely, the adverse effects of) an autoimmune disease, such as chronic inflammatory bowel disease, systemic lupus erythematosus, psoriasis, muckle-wells syndrome, rheumatoid arthritis, multiple sclerosis, or Hashimoto's disease: an allergic disease, such as a food allergy, pollenosis, or asthma: an infectious disease, such as an infection with Clostridium difficile: an inflammatory disease such as a TNF-mediated inflammatory disease (e.g., an inflammatory disease of the gastrointestinal tract, such as pouchitis, a cardiovascular inflammatory condition, such as atherosclerosis, or an inflammatory lung disease, such as chronic obstructive pulmonary disease): a pharmaceutical composition for suppressing rejection in organ transplantation or other situations in which tissue rejection might occur: a supplement, food, or beverage for improving immune functions; or a reagent for suppressing the proliferation or function of immune cells.

In some embodiments, the methods provided herein are useful for the treatment of inflammation. In certain embodiments, the inflammation of any tissue and organs of the body, including musculoskeletal inflammation, vascular inflammation, neural inflammation, digestive system inflammation, ocular inflammation, inflammation of the reproductive system, and other inflammation, as discussed below. In some embodiments, the inflammation comprises a Th1 mediated inflammation. In some embodiments, the inflammation comprises a Th2 mediated inflammation (such as with asthma or atopic dermatitis). In some embodiments, the inflammation comprises a Th17 mediated inflammation (such as with psoriasis).

Immune disorders of the musculoskeletal system include, but are not limited, to those conditions affecting skeletal joints, including joints of the hand, wrist, elbow, shoulder, jaw, spine, neck, hip, knew, ankle, and foot, and conditions affecting tissues connecting muscles to bones such as tendons. Examples of such immune disorders, which may be treated with the methods and compositions described herein include, but are not limited to, arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

In some embodiments, the methods provided herein are useful for the treatment of psoriasis.

In some embodiments, the methods provided herein are useful for the treatment of atopic dermatitis.

Ocular immune disorders refers to a immune disorder that affects any structure of the eye, including the eye lids. Examples of ocular immune disorders which may be treated with the methods and compositions described herein include, but are not limited to, blepharitis, blepharochalasis, conjunctivitis, dacryoadenitis, keratitis, keratoconjunctivitis sicca (dry eye), scleritis, trichiasis, and uveitis.

Examples of nervous system immune disorders which may be treated with the methods and compositions described herein include, but are not limited to, encephalitis, Guillain-Barre syndrome, meningitis, neuromyotonia, narcolepsy, multiple sclerosis, myelitis and schizophrenia. Examples of inflammation of the vasculature or lymphatic system which may be treated with the methods and compositions described herein include, but are not limited to, arthrosclerosis, arthritis, phlebitis, vasculitis, and lymphangitis.

Examples of digestive system immune disorders which may be treated with the methods and therapeutic compositions described herein include, but are not limited to, cholangitis, cholecystitis, enteritis, enterocolitis, gastritis, gastroenteritis, inflammatory bowel disease, ileitis, and proctitis. Inflammatory bowel diseases include, for example, certain art-recognized forms of a group of related conditions. Several major forms of inflammatory bowel diseases are known, with Crohn's disease (regional bowel disease, e.g., inactive and active forms) and ulcerative colitis (e.g., inactive and active forms) the most common of these disorders. In addition, the inflammatory bowel disease encompasses irritable bowel syndrome, microscopic colitis, lymphocytic-plasmocytic enteritis, celiac disease, collagenous colitis, lymphocytic colitis and eosinophilic enterocolitis. Other less common forms of IBD include indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, and primary sclerosing cholangitis.

Examples of reproductive system immune disorders which may be treated with the methods and therapeutic compositions described herein include, but are not limited to, cervicitis, chorioamnionitis, endometritis, epididymitis, omphalitis, oophoritis, orchitis, salpingitis, tubo-ovarian abscess, urethritis, vaginitis, vulvitis, and vulvodynia.

The methods and therapeutic compositions described herein may be used to treat autoimmune conditions having an inflammatory component. Such conditions include, but are not limited to, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, good pasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, Ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, ulcerative colitis, and vitiligo.

The methods and therapeutic compositions described herein may be used to treat T-cell mediated hypersensitivity diseases having an inflammatory component. Such conditions include, but are not limited to, contact hypersensitivity, contact dermatitis (including that due to poison ivy), urticaria, skin allergies, respiratory allergies (hay fever, allergic rhinitis, house dust mite allergy) and gluten-sensitive enteropathy (celiac disease).

Other immune disorders which may be treated with the methods and therapeutic compositions include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, and stomatitis, transplant rejection (involving organs such as kidney, liver, heart, lung, pancreas (e.g., islet cells), bone marrow, cornea, small bowel, skin allografts, skin homografts, and heart valve xenografts, serum sickness, and graft vs host disease), acute pancreatitis, chronic pancreatitis, acute respiratory distress syndrome, Sexary's syndrome, congenital adrenal hyperplasia, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenia purpura in adults, secondary thrombocytopenia in adults, acquired (autoimmune) haemolytic anemia, leukemia and lymphomas in adults, acute leukemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosus, psoriasis, chronic obstructive pulmonary disease, and inflammation accompanying infectious conditions (e.g., sepsis).

### Metabolic Disorders

In some embodiments, the methods and therapeutic compositions described herein relate to the treatment or prevention of a metabolic disease or disorder a, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglyceridemia, ketoacidosis, hypoglycemia, thrombotic disorders, dyslipidemia, non-alcoholic fatty liver disease (NAFLD), nonalcoholic steatohepatitis (NASH) or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema. In some embodiments, the methods and therapeutic compositions described herein relate to the treatment of NAFLD and NASH.

The methods described herein can be used to treat any subject in need thereof. As used herein, a “subject in need thereof” includes any subject that has a metabolic disease or disorder, as well as any subject with an increased likelihood of acquiring a such a disease or disorder.

The therapeutic compositions described herein can be used, for example, for preventing or treating (reducing, partially or completely, the adverse effects of) a metabolic disease, such as type II diabetes, impaired glucose tolerance, insulin resistance, obesity, hyperglycemia, hyperinsulinemia, fatty liver, non-alcoholic steatohepatitis, hypercholesterolemia, hypertension, hyperlipoproteinemia, hyperlipidemia, hypertriglyceridemia, ketoacidosis, hypoglycemia, thrombotic disorders, dyslipidemia, NAFLD, NASH, or a related disease. In some embodiments, the related disease is cardiovascular disease, atherosclerosis, kidney disease, nephropathy, diabetic neuropathy, diabetic retinopathy, sexual dysfunction, dermatopathy, dyspepsia, or edema.

### Other Diseases and Disorders

In some embodiments, the methods and therapeutic compositions described herein relate to the treatment of liver diseases. Such diseases include, but are not limited to, Alagille syndrome, alcohol-related liver disease, alpha-1 antitrypsin deficiency, autoimmune hepatitis, biliary atresia, cirrhosis, galactosemia, Gilbert's syndrome, hemochromatosis, hepatitis A, hepatitis B, hepatitis C, hepatic encephalopathy, intrahepatic cholestasis of pregnancy (ICP), lysosomal acid lipase deficiency (LAL-D), Liver cysts, newborn jaundice, primary biliary cholangitis (PBC), primary sclerosing cholangitis (PSC), Reye syndrome, type I glycogen storage disease, and Wilson's disease.

The methods and therapeutic compositions described herein may be used to treat neurodegenerative and neurological diseases. In certain embodiments, the neurodegenerative and/or neurological disease is Parkinson's disease, Alzheimer's disease, prion disease, Huntington's disease, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy or post-operative cognitive dysfunction.

### Dysbiosis

The gut microbiome (also called the “gut microbiota”) can have a significant impact on an individual's health through microbial activity and influence (local and/or distal) on immune and other cells of the host (Walker, W. A., Dysbiosis. The Microbiota in Gastrointestinal Pathophysiology. Chapter 25. 2017: Weiss and Thierry, Mechanisms and consequences of intestinal dysbiosis. Cellular and Molecular Life Sciences. (2017) 74 (16): 2959-2977. Zurich Open Repository and Archive, doi.org/10.1007/s00018-017-2509-x)).

A healthy host-gut microbiome homeostasis is sometimes referred to as a “eubiosis” or “normobiosis,” whereas a detrimental change in the host microbiome composition and/or its diversity can lead to an unhealthy imbalance in the microbiome, or a “dysbiosis” (Hooks and O'Malley. Dysbiosis and its discontents. American Society for Microbiology. October 2017. Vol. 8. Issue 5. mBio 8:e01492-17. doi.org/10.1128/mBio.01492-17). Dysbiosis, and associated local or distal host inflammatory or immune effects, may occur where microbiome homeostasis is lost or diminished, resulting in: increased susceptibility to pathogens; altered host bacterial metabolic activity: induction of host proinflammatory activity and/or reduction of host anti-inflammatory activity. Such effects are mediated in part by interactions between host immune cells (e.g., T cells, dendritic cells, mast cells, NK cells, intestinal epithelial lymphocytes (IEC), macrophages and phagocytes) and cytokines, and other substances released by such cells and other host cells.

A dysbiosis may occur within the gastrointestinal tract (a “gastrointestinal dysbiosis” or “gut dysbiosis”) or may occur outside the lumen of the gastrointestinal tract (a “distal dysbiosis”). Gastrointestinal dysbiosis is often associated with a reduction in integrity of the intestinal epithelial barrier, reduced tight junction integrity and increased intestinal permeability. Citi, S. Intestinal Barriers protect against disease, Science 359:1098-99 (2018): Srinivasan et al., TEER measurement techniques for in vitro barrier model systems. J. Lab. Autom. 20:107-126 (2015). A gastrointestinal dysbiosis can have physiological and immune effects within and outside the gastrointestinal tract.

The presence of a dysbiosis can be associated with a wide variety of diseases and conditions including: infection, cancer, autoimmune disorders (e.g., systemic lupus erythematosus (SLE)) or inflammatory disorders (e.g., functional gastrointestinal disorders such as inflammatory bowel disease (IBD), ulcerative colitis, and Crohn's disease), neuroinflammatory diseases (e.g., multiple sclerosis), transplant disorders (e.g., graft-versus-host disease), fatty liver disease, type I diabetes, rheumatoid arthritis, Sjögren's syndrome, celiac disease, cystic fibrosis, chronic obstructive pulmonary disorder (COPD), and other diseases and conditions associated with immune dysfunction. Lynch et al., The Human Microbiome in Health and Disease, N. Engl. J. Med. 375:2369-79 (2016), Carding et al., Dysbiosis of the gut microbiota in disease. Microb. Ecol. Health Dis. (2015): 26: 10: 3402/mehd.v26.2619; Levy et al, Dysbiosis and the Immune System, Nature Reviews Immunology 17:219 (April 2017)

In certain embodiments, exemplary therapeutic compositions disclosed herein can treat a dysbiosis and its effects by modifying the immune activity present at the site of dysbiosis. As described herein, such compositions can modify a dysbiosis via effects on host immune cells, resulting in, e.g., an increase in secretion of anti-inflammatory cytokines and/or a decrease in secretion of pro-inflammatory cytokines, reducing inflammation in the subject recipient or via changes in metabolite production.

Exemplary therapeutic compositions disclosed herein that are useful for treatment of disorders associated with a dysbiosis contain Prevotella histicola EVs. Such compositions are capable of affecting the recipient host's immune function, in the gastrointestinal tract, and/or a systemic effect at distal sites outside the subject's gastrointestinal tract.

Exemplary therapeutic compositions disclosed herein that are useful for treatment of disorders associated with a dysbiosis contain a population of Prevotella histicola EVs. Such compositions are capable of affecting the recipient host's immune function, in the gastrointestinal tract, and/or a systemic effect at distal sites outside the subject's gastrointestinal tract.

In one embodiment, therapeutic compositions containing an isolated population of EVs derived from Prevotella histicola bacteria are administered (e.g., orally) to a mammalian recipient in an amount effective to treat a dysbiosis and one or more of its effects in the recipient. The dysbiosis may be a gastrointestinal tract dysbiosis or a distal dysbiosis.

In another embodiment, therapeutic compositions of the instant invention can treat a gastrointestinal dysbiosis and one or more of its effects on host immune cells, resulting in an increase in secretion of anti-inflammatory cytokines and/or a decrease in secretion of pro-inflammatory cytokines, reducing inflammation in the subject recipient.

In another embodiment, the therapeutic compositions can treat a gastrointestinal dysbiosis and one or more of its effects by modulating the recipient immune response via cellular and cytokine modulation to reduce gut permeability by increasing the integrity of the intestinal epithelial barrier.

In another embodiment, the therapeutic compositions can treat a distal dysbiosis and one or more of its effects by modulating the recipient immune response at the site of dysbiosis via modulation of host immune cells.

Other exemplary therapeutic compositions are useful for treatment of disorders associated with a dysbiosis, which compositions contain one or more types of bacteria and/or EVs capable of altering the relative proportions of host immune cell subpopulations, e.g., subpopulations of T cells, immune lymphoid cells, dendritic cells, NK cells and other immune cells, or the function thereof, in the recipient.

Other exemplary therapeutic compositions are useful for treatment of disorders associated with a dysbiosis, which compositions contain a population of Prevotella histicola EVs capable of altering the relative proportions of immune cell subpopulations, e.g., T cell subpopulations, immune lymphoid cells, NK cells and other immune cells, or the function thereof, in the recipient subject.

In one embodiment, the invention provides methods of treating a gastrointestinal dysbiosis and one or more of its effects by orally administering to a subject in need thereof a therapeutic composition that alters the microbiome population existing at the site of the dysbiosis. The therapeutic composition can contain Prevotella histicola EVs.

In one embodiment, the invention provides methods of treating a distal dysbiosis and one or more of its effects by orally administering to a subject in need thereof a therapeutic composition that alters the subject's immune response outside the gastrointestinal tract. The therapeutic composition can contain one or more types of EVs from immunomodulatory bacteria or a population of Prevotella histicola EVs.

In exemplary embodiments, therapeutic compositions useful for treatment of disorders associated with a dysbiosis stimulate secretion of one or more anti-inflammatory cytokines by host immune cells. Anti-inflammatory cytokines include, but are not limited to, IL-10, IL-13, IL-9, IL-4, IL-5, TGFβ, and combinations thereof. In other exemplary embodiments, therapeutic compositions useful for treatment of disorders associated with a dysbiosis that decrease (e.g., inhibit) secretion of one or more pro-inflammatory cytokines by host immune cells. Pro-inflammatory cytokines include, but are not limited to, IFNγ, IL-12p70, IL-1α, IL-6, IL-8, MCP1, MIP1α, MIP1β, TNFα, and combinations thereof. Other exemplary cytokines are known in the art and are described herein.

In another aspect, the invention provides a method of treating or preventing a disorder associated with a dysbiosis in a subject in need thereof, comprising administering (e.g., orally administering) to the subject a therapeutic composition in the form of a probiotic or medical food comprising Prevotella histicola EVs in an amount sufficient to alter the microbiome at a site of the dysbiosis, such that the disorder associated with the dysbiosis is treated.

In another embodiment, a therapeutic composition of the instant invention in the form of a probiotic or medical food may be used to prevent or delay the onset of a dysbiosis in a subject at risk for developing a dysbiosis.

### Methods of Making Enhanced Bacteria

In certain aspects, provided herein are methods of making engineered bacteria for the production of the Prevotella histicola EVs described herein. In some embodiments, the engineered bacteria are modified to enhance certain desirable properties. For example, in some embodiments, the engineered bacteria are modified to enhance the immunomodulatory and/or therapeutic effect of the EVs (e.g., either alone or in combination with another therapeutic agent), to reduce toxicity and/or to improve bacterial and/or EV manufacturing (e.g., higher oxygen tolerance, improved freeze-thaw tolerance, shorter generation times). The engineered bacteria may be produced using any technique known in the art, including but not limited to site-directed mutagenesis, transposon mutagenesis, knock-outs, knock-ins, polymerase chain reaction mutagenesis, chemical mutagenesis, ultraviolet light mutagenesis, transformation (chemically or by electroporation), phage transduction, directed evolution, CRISPR/Cas9, or any combination thereof.

In some embodiments of the methods provided herein, the bacterium is modified by directed evolution. In some embodiments, the directed evolution comprises exposure of the bacterium to an environmental condition and selection of bacterium with improved survival and/or growth under the environmental condition. In some embodiments, the method comprises a screen of mutagenized bacteria using an assay that identifies enhanced bacterium. In some embodiments, the method further comprises mutagenizing the bacteria (e.g., by exposure to chemical mutagens and/or UV radiation) or exposing them to a therapeutic agent (e.g., antibiotic) followed by an assay to detect bacteria having the desired phenotype (e.g., an in vivo assay, an ex vivo assay, or an in vitro assay).

### EXAMPLES

### Example 1: Preparation of Lyophilates

The excipient stocks with the formulas provided in Table A to Table D were prepared (amounts shown are percentages of each component in the formula) as solutions. The formulas of the excipient stock fall in to 2 main categories: with and without polymers. The excipient stock solutions were mixed with a liquid preparation of extracellular vesicles. The resulting solutions were freeze-dried and analyzed.

In this Example, the extracellular vesicles (smEVs) used in the studies were isolated from a strain of Prevotella histicola (Prevotella Strain B) (NRRL accession number B 50329).

The data collected from the lyophilization of these mixtures are provided in Table E. All measured samples had a residual moisture content of less than 5%. Some samples were additionally tested in vivo using keyhole limpet hemocyanin (KLH)-specific inflammation in a delayed-type hypersensitivity (DTH) model. The samples tested in KLH-DTH showed efficacy.

TABLE A
Stocks comprising excipients for stabilizing extracellular
vesicles during lyophilization. The numerical values given
are on a weight percent basis in the solution.

| | | | | | | Malto- |
|---|---|---|---|---|---|---|
| Formula | Sucrose | Trehalose | Mannitol | Sorbitol | Dextran | dextrin |
| 1 | 40 | 15 | 20 | | 25 | |
| 2 | 20 | 20 | 50 | | 10 | |
| 3 | 50 | | 50 | | | |
| 4 | 40 | 10 | 50 | | | |
| 5 | | 10 | 70 | 0.5 | 19.5 | |
| 7 | | 19.5 | 80 | 0.5 | | |
| 7a | | 20 | 80 | | | |
| 7e | 27 | 20 | 53 | | | |
| 8 | | 10 | 75 | | 15 | |
| 15 | | 19.5 | 70 | 0.5 | 10 | |
| 16 | | 19.5 | 75 | 0.5 | 5 | |
| 17 | | 20 | 80 | | | |
| 18 | | 10 | 60 | | 30 | |
| 19 | | 10 | | | 30 | 60 |
| 20 | | | 100 | | | |

TABLE B
Stocks comprising excipients including polymers for stabilizing
extracellular vesicles during lyophilization. The numerical
values given are on a weight percent basis in the solution.

| Formula | Sucrose | PVP-K30 | Ficoll | Citrate | Arginine |
|---|---|---|---|---|---|
| 6 | 20 | 78 | | 1 | 1 |
| 14 | 20 | | 78 | 1 | 1 |

TABLE C
Stocks comprising excipients including polymers for stabilizing
extracellular vesicles during lyophilization. The numerical
values given are on a weight percent basis in the solution.

| | | | Man- | PVP- | Hydroxypropyl- | |
|---|---|---|---|---|---|---|
| Formula | Sucrose | Trehalose | nitol | K30 | B-cyclodextrin | Ficoll |
| 9 | 20 | 10 | 50 | 20 | | |
| 10 | 10 | 10 | 50 | | 30 | |
| 13 | 20 | 10 | 50 | | | 20 |

TABLE D
Stocks comprising excipients including polymers for stabilizing
extracellular vesicles during lyophilization. The numerical
values given are on a weight percent basis in the solution.

| Formula | Mannitol | Poloxamer 188 |
|---|---|---|
| 11 | 95 | 5 |
| 12 | 90 | 10 |

TABLE E
Analytical data obtained for excipient stock solutions used
for the stabilization of extracellular vesicles. “% Stabilizer”
refers to the percentage of the stock solution formula that was added
to a liquid preparation of EVs on a weight basis. “% Moisture”
was determined by Karl Fischer titration. Zₐᵥₑ was determined
by dynamic light scattering (DLS). For particles per mass,
particle numbers were determined by Z-view or NTA instrument;
mass (mg) were decided by analytical balance.

| | % | % | Zave, | Particles per mass, |
|---|---|---|---|---|
| Formula | Stabilizer | Moisture | nm | p/mg |
| — | 0% | | 226.1 | 6.45E+11 |
| 4 | 34% | 2 | 206.2 | 6.28E+10 |
| 5 | 41% | | 209.1 | 6.76E+10 |
| 6 | 35% | 3.6 | 212.8 | 3.25E+10 |
| 7 | 47% | 2.7 | 204 | 7.02E+10 |
| 8 | 44% | 3 | 206.4 | 6.99E+10 |
| 9 | 34% | 2.5 | 187.3 | 7.15E+10 |
| 10 | 34% | 2.7 | 180.1 | 7.37E+10 |
| 11 | 56% | 1.8 | 205.2 | 7.08E+10 |
| 12 | 53% | 1.8 | 202 | 7.66E+10 |
| 13 | 30% | 3 | 172.3 | 7.77E+10 |
| 14 | 35% | 3.8 | 137.4 | 6.12E+10 |
| 15 | 41% | 2.9 | 205.8 | |
| 16 | 44% | 2.8 | 203.9 | |

### Lyophilization Cycle for Extracellular Vesicles (EVs)

The lyophilization cycle is optimized for each excipient formulation. Differences in the critical temperature and collapse temperature of the mixtures mean the shelf temperature during lyophilization is adjusted accordingly. The optimization process involves 3 steps: initial screening, primary drying optimization, and secondary drying optimization. The final cycle is confirmed to be sufficient to dry the material below 5% residual moisture. In this example, the excipient formula chosen for optimization was excipient formula 7.

TABLE F

| SHELF TEMP (° C.) | SAMPLE TEMP (° C.) |
|---|---|
| −5 | −17.9 |
| −15 | −23.6 |
| −20 | −26.3 |
| −25 | −28.9 |

TABLE G

| | | | |
|---|---|---|---|
| −25 | 2.9 | 31.5 | 189 |
| −20 | 2.8 | 28.2 | 215 |
| −15 | 3.3 | 20.8 | 224 |
| −5 | 1.5 | 18.2 | 202 |

TABLE H

| | | |
|---|---|---|
| Primary Drying | 2.8 | 2 |
| Secondary Drying | 2.6 | 29 |

TABLE I
Final lyophilization cycle optimized for extracellular vesicles
stabilized with 47% (by volume) of excipient formula 7.

| | | | | |
|---|---|---|---|---|
| Freezing (from RT) | −45 | 200 | 10 | 600K |
| Primary Drying | −20 | 75 | 2,151 | 300 |
| Secondary Drying | 25 | 126 | 300 | 300 |
| Hold | 25 | 0 | 0-300 | 300 |

### Example 2: Orally-Delivered Microbial Extracellular Vesicle Induces Anti-Inflammatory Activity in Mice

Significantly decreased ear swelling and inflammation were observed in a delayed-type hypersensitivity model, showing that extracellular vesicles (EVs) from Prevotella histicola Strain B (NRRL accession number B 50329) modulate systemic inflammatory responses. Activity of the Prevotella histicola EVs is dependent upon both TLR2 signaling and the presence of local immune cells. In vitro results show TLR2 agonism and induction of anti-inflammatory cytokine responses in immune cells by the EVs. These findings demonstrate the anti-inflammatory effects of an orally delivered microbial extracellular vesicle. The Prevotella histicola EVs induce broad-based resolution of inflammation across multiple pathways via a novel mechanism of systemic pharmacology without systemic exposure. EVs are particularly effective at engaging host cells in the gut to modulate distal inflammation. These data point to oral EVs as a new class of immunotherapeutic drugs.

### Example 3: Purification and Preparation of Extracellular Vesicles (EVs) from Bacteria

### Purification

Extracellular vesicles (EVs) are purified and prepared from bacterial cultures using methods known to those skilled in the art (S. Bin Park, et al. PLoS ONE. 6 (3):e17629 (2011)).

For example, bacterial cultures are centrifuged at 10,000-15,500×g for 10-40 min at 4° C. or room temperature to pellet bacteria. Culture supernatants are then filtered to include material ≤0.22 μm (for example, via a 0.22 μm or 0.45 μm filter) and to exclude intact bacterial cells. Filtered supernatants are concentrated using methods that may include, but are not limited to, ammonium sulfate precipitation, ultracentrifugation, or filtration. Briefly, for ammonium sulfate precipitation, 1.5-3 M ammonium sulfate is added to filtered supernatant slowly, while stirring at 4° C. Precipitations are incubated at 4° C. for 8-48 hours and then centrifuged at 11,000×g for 20-40 min at 4° C. The pellets contain EVs and other debris. Briefly, using ultracentrifugation, filtered supernatants are centrifuged at 100,000-200,000×g for 1-16 hours at 4° C. The pellet of this centrifugation contains EVs and other debris. Briefly, using a filtration technique, using an Amicon Ultra spin filter or by tangential flow filtration, supernatants are filtered so as to retain species of molecular weight >50, 100, 300, or 500 kDa.

Alternatively, EVs are obtained from bacterial cultures continuously during growth, or at selected time points during growth, by connecting a bioreactor to an alternating tangential flow (ATF) system (e.g., XCell ATF from Repligen) according to manufacturer's instructions. The ATF system retains intact cells (>0.22 μm) in the bioreactor, and allows smaller components (e.g., EVs, free proteins) to pass through a filter for collection. For example, the system may be configured so that the <0.22 μm filtrate is then passed through a second filter of 100 kDa, allowing species such as EVs between 0.22 μm and 100 kDa to be collected, and species smaller than 100 kDa to be pumped back into the bioreactor. Alternatively, the system may be configured to allow for medium in the bioreactor to be replenished and/or modified during growth of the culture. EVs collected by this method may be further purified and/or concentrated by ultracentrifugation or filtration as described above for filtered supernatants.

EVs obtained by methods described above may be further purified by gradient ultracentrifugation, using methods that may include, but are not limited to, use of a sucrose gradient or Optiprep gradient. Briefly, using a sucrose gradient method, if ammonium sulfate precipitation or ultracentrifugation were used to concentrate the filtered supernatants, pellets are resuspended in 60% sucrose, 30 mM Tris, pH 8.0. If filtration was used to concentrate the filtered supernatant, the concentrate is buffer exchanged into 60% sucrose, 30 mM Tris, pH 8.0, using an Amicon Ultra column. Samples are applied to a 35-60% discontinuous sucrose gradient and centrifuged at 200,000×g for 3-24 hours at 4° C. Briefly, using an Optiprep gradient method, if ammonium sulfate precipitation or ultracentrifugation were used to concentrate the filtered supernatants, pellets are resuspended in 45% Optiprep in PBS. If filtration was used to concentrate the filtered supernatant, the concentrate is diluted using 60% Optiprep to a final concentration of 45% Optiprep. Samples are applied to a 0-45% discontinuous sucrose gradient and centrifuged at 200,000×g for 3-24 hours at 4° C. Alternatively, high resolution density gradient fractionation could be used to separate EVs based on density.

### Preparation

To confirm sterility and isolation of the EV preparations, EVs are serially diluted onto agar medium used for routine culture of the bacteria being tested and incubated using routine conditions. Non-sterile preparations are passed through a 0.22 μm filter to exclude intact cells. To further increase purity, isolated EVs may be DNase or proteinase K treated.

Alternatively, for preparation of EVs used for in vivo injections, purified EVs are processed as described previously (G. Norheim, et al. PLoS ONE. 10 (9): e0134353 (2015)). Briefly, after sucrose gradient centrifugation, bands containing EVs are resuspended to a final concentration of 50 g/mL in a solution containing 3% sucrose or other solution suitable for in vivo injection known to one skilled in the art. This solution may also contain adjuvant, for example aluminum hydroxide at a concentration of 0-0.5% (w/v).

To make samples compatible with further testing (e.g., to remove sucrose prior to TEM imaging or in vitro assays), samples are buffer exchanged into PBS or 30 mM Tris, pH 8.0 using filtration (e.g., Amicon Ultra columns), dialysis, or ultracentrifugation (following 15-fold or greater dilution in PBS, 200,000×g, 1-3 hours, 4° C.) and resuspension in PBS.

For all of these studies, EVs may be heated, irradiated, and/or lyophilized prior to administration (as described herein).

### Example 4: Manipulating Bacteria Through Stress to Produce Various Amounts of EVs and/or to Vary Content of EVs

Stress, and in particular envelope stress, has been shown to increase production of EVs by some bacterial strains (I. MacDonald, M. Kuehn. J Bacteriol 195 (13): doi: 10/1128/JB.02267-12). In order to vary production of EVs by bacteria, bacteria are stressed using various methods.

Bacteria may be subjected to single stressors or stressors in combination. The effects of different stressors on different bacteria is determined empirically by varying the stress condition and determining the IC50 value (the conditions required to inhibit cell growth by 50%). EV purification, quantification, and characterization occurs. EV production is quantified (1) in complex samples of bacteria and EVs by nanoparticle tracking analysis (NTA) or transmission electron microscopy (TEM); or (2) following EV purification by NTA, lipid quantification, or protein quantification. EV content is assessed following purification by methods described above.

### Antibiotic Stress

Bacteria are cultivated under standard growth conditions with the addition of sublethal concentrations of antibiotics. This may include 0.1-1 μg/mL chloramphenicol, or 0.1-0.3 μg/mL gentamicin, or similar concentrations of other antibiotics (e.g., ampicillin, polymyxin B). Host antimicrobial products such as lysozyme, defensins, and Reg proteins may be used in place of antibiotics. Bacterially-produced antimicrobial peptides, including bacteriocins and microcins may also be used.

### Temperature Stress

Bacteria are cultivated under standard growth conditions, but at higher or lower temperatures than are typical for their growth. Alternatively, bacteria are grown under standard conditions, and then subjected to cold shock or heat shock by incubation for a short period of time at low or high temperatures respectively. For example, bacteria grown at 37° C. are incubated for 1 hour at 4° C.-18° C. for cold shock or 42° C.-50° C. for heat shock.

### Starvation and Nutrient Limitation

To induce nutritional stress, bacteria are cultivated under conditions where one or more nutrients are limited. Bacteria may be subjected to nutritional stress throughout growth or shifted from a rich medium to a poor medium. Some examples of media components that are limited are carbon, nitrogen, iron, and sulfur. An example medium is M9 minimal medium (Sigma-Aldrich), which contains low glucose as the sole carbon source. Particularly for Prevotella spp . . . iron availability is varied by altering the concentration of hemin in media and/or by varying the type of porphyrin or other iron carrier present in the media, as cells grown in low hemin conditions were found to produce greater numbers of EVs (S. Stubbs et al. Letters in Applied Microbiology. 29:31-36 (1999). Media components are also manipulated by the addition of chelators such as EDTA and deferoxamine.

### Saturation

Bacteria are grown to saturation and incubated past the saturation point for various periods of time. Alternatively, conditioned media is used to mimic saturating environments during exponential growth. Conditioned media is prepared by removing intact cells from saturated cultures by centrifugation and filtration, and conditioned media may be further treated to concentrate or remove specific components.

### Salt Stress

Bacteria are cultivated in or exposed for brief periods to medium containing NaCl, bile salts, or other salts.

### UV Stress

UV stress is achieved by cultivating bacteria under a UV lamp or by exposing bacteria to UV using an instrument such as a Stratalinker (Agilent). UV may be administered throughout the entire cultivation period, in short bursts, or for a single defined period following growth.

### Reactive Oxygen Stress

Bacteria are cultivated in the presence of sublethal concentrations of hydrogen peroxide (250-1,000 μM) to induce stress in the form of reactive oxygen species. Anaerobic bacteria are cultivated in or exposed to concentrations of oxygen that are toxic to them.

### Detergent Stress

Bacteria are cultivated in or exposed to detergent, such as sodium dodecyl sulfate (SDS) or deoxycholate.

### pH Stress

Bacteria are cultivated in or exposed for limited times to media of different pH.

### Example 5: Profiling EV Composition and Content

EVs may be characterized by any one of various methods including, but not limited to, NanoSight characterization, SDS-PAGE gel electrophoresis, Western blot, ELISA, liquid chromatography-mass spectrometry and mass spectrometry, dynamic light scattering, lipid levels, total protein, lipid to protein ratios, nucleic acid analysis and/or zeta potential.

### NanoSight Characterization of EVs

Nanoparticle tracking analysis (NTA) is used to characterize the size distribution of purified EVs. Purified EV preparations are run on a NanoSight machine (Malvern Instruments) to assess EV size and concentration.

### SDS-PAGE Gel Electrophoresis

To identify the protein components of purified EVs, samples are run on a gel, for example a Bolt Bis-Tris Plus 4-12% gel (Thermo-Fisher Scientific), using standard techniques. Samples are boiled in 1×SDS sample buffer for 10 minutes, cooled to 4° C., and then centrifuged at 16,000×g for 1 min. Samples are then run on a SDS-PAGE gel and stained using one of several standard techniques (e.g., Silver staining, Coomassie Blue, Gel Code Blue) for visualization of bands.

### Western Blot Analysis

To identify and quantify specific protein components of purified EVs, EV proteins are separated by SDS-PAGE as described above and subjected to Western blot analysis (Cvjetkovic et al., Sci. Rep. 6, 36338 (2016)) and are quantified via ELISA.

### EV Proteomics and Liquid Chromatography-Mass Spectrometry (LC-MS/MS) and Mass Spectrometry (MS)

Proteins present in EVs are identified and quantified by Mass Spectrometry techniques. EV proteins may be prepared for LC-MS/MS using standard techniques including protein reduction using dithiothreitol solution (DTT) and protein digestion using enzymes such as LysC and trypsin as described in Erickson et al, 2017 (Molecular Cell, VOLUME 65, ISSUE 2, P361-370, Jan. 19, 2017). Alternatively, peptides are prepared as described by Liu et al. 2010 (JOURNAL OF BACTERIOLOGY, June 2010, p. 2852-2860 Vol. 192, No. 11), Kieselbach and Oscarsson 2017 (Data Brief. 2017 February; 10: 426-431), Vildhede et al, 2018 (Drug Metabolism and Disposition Feb. 8, 2018). Following digestion, peptide preparations are run directly on liquid chromatography and mass spectrometry devices for protein identification within a single sample. For relative quantitation of proteins between samples, peptide digests from different samples are labeled with isobaric tags using the iTRAQ Reagent-8plex Multiplex Kit (Applied Biosystems, Foster City, CA) or TMT 10plex and 11plex Label Reagents (Thermo Fischer Scientific, San Jose, CA, USA). Each peptide digest is labeled with a different isobaric tag and then the labeled digests are combined into one sample mixture. The combined peptide mixture is analyzed by LC-MS/MS for both identification and quantification. A database search is performed using the LC-MS/MS data to identify the labeled peptides and the corresponding proteins. In the case of isobaric labeling, the fragmentation of the attached tag generates a low molecular mass reporter ion that is used to obtain a relative quantitation of the peptides and proteins present in each EV.

Additionally, metabolic content is ascertained using liquid chromatography techniques combined with mass spectrometry. A variety of techniques exist to determine metabolomic content of various samples and are known to one skilled in the art involving solvent extraction, chromatographic separation and a variety of ionization techniques coupled to mass determination (Roberts et al 2012 Targeted Metabolomics. Curr Protoc Mol Biol. 30:1-24; Dettmer et al 2007, Mass spectrometry-based metabolomics. Mass Spectrom Rev. 26 (1): 51-78). As a non-limiting example, a LC-MS system includes a 4000 QTRAP triple quadrupole mass spectrometer (AB SCIEX) combined with 1100 Series pump (Agilent) and an HTS PAL autosampler (Leap Technologies). Media samples or other complex metabolic mixtures (˜10 μL) are extracted using nine volumes of 74.9:24.9:0.2 (v/v/v) acetonitrile/methanol/formic acid containing stable isotope-labeled internal standards (valine-d8, Isotec; and phenylalanine-d8, Cambridge Isotope Laboratories). Standards may be adjusted or modified depending on the metabolites of interest. The samples are centrifuged (10 minutes, 9,000×g, 4° C.), and the supernatants (10 μL) are submitted to LCMS by injecting the solution onto the HILIC column (150×2.1 mm, 3 μm particle size). The column is eluted by flowing a 5% mobile phase [10 mM ammonium formate, 0.1% formic acid in water] for 1 minute at a rate of 250 μL/minute followed by a linear gradient over 10 minutes to a solution of 40% mobile phase [acetonitrile with 0.1% formic acid]. The ion spray voltage is set to 4.5 kV and the source temperature is 450° C.

The data are analyzed using commercially available software like Multiquant 1.2 from AB SCIEX for mass spectrum peak integration. Peaks of interest should be manually curated and compared to standards to confirm the identity of the peak. Quantitation with appropriate standards is performed to determine the number of metabolites present in the initial media, after bacterial conditioning. A non-targeted metabolomics approach may also be used using metabolite databases, such as but not limited to the NIST database, for peak identification.

### Dynamic Light Scattering (DLS)

DLS measurements, including the distribution of particles of different sizes in different EV preparations are taken using instruments such as the DynaPro NanoStar (Wyatt Technology) and the Zetasizer Nano ZS (Malvern Instruments).

### Lipid Levels

Lipid levels are quantified using FM4-64 (Life Technologies), by methods similar to those described by A. J. McBroom et al. J Bacteriol 188:5385-5392. and A. Frias, et al. Microb Ecol. 59:476-486 (2010). Samples are incubated with FM4-64 (3.3 μg/mL in PBS for 10 minutes at 37° C. in the dark). After excitation at 515 nm, emission at 635 nm is measured using a Spectramax M5 plate reader (Molecular Devices). Absolute concentrations are determined by comparison of unknown samples to standards (such as palmitoyloleyolphosphatidylglycerol (POPG) vesicles) of known concentrations. Lipidomics can be used to identify the lipids present in the EVs.

### Total Protein

Protein levels are quantified by standard assays such as the Bradford and BCA assays. The Bradford assays are run using Quick Start Bradford 1× Dye Reagent (Bio-Rad), according to manufacturer's protocols. BCA assays are run using the Pierce BCA Protein Assay Kit (Thermo-Fisher Scientific). Absolute concentrations are determined by comparison to a standard curve generated from BSA of known concentrations. Alternatively, protein concentration can be calculated using the Beer-Lambert equation using the sample absorbance at 280 nm (A280) as measured on a Nanodrop spectrophotometer (Thermo-Fisher Scientific). In addition, proteomics may be used to identify proteins in the sample.

### Lipid:Protein Ratios

Lipid:protein ratios are generated by dividing lipid concentrations by protein concentrations. These provide a measure of the purity of vesicles as compared to free protein in each preparation.

### Nucleic Acid Analysis

Nucleic acids are extracted from EVs and quantified using a Qubit fluorimeter. Size distribution is assessed using a BioAnalyzer, and the material is sequenced.

### Zeta Potential

The zeta potential of different preparations are measured using instruments such as the Zetasizer ZS (Malvern Instruments).

### Example 6: Manufacturing Conditions

Enriched media is used to grow and prepare the bacteria for in vitro and in vivo use and, ultimately, for EV preparations. For example, media may contain sugar, yeast extracts, plant-based peptones, buffers, salts, trace elements, surfactants, anti-foaming agents, and vitamins. Composition of complex components such as yeast extracts and peptones may be undefined or partially defined (including approximate concentrations of amino acids, sugars etc.). Microbial metabolism may be dependent on the availability of resources such as carbon and nitrogen. Various sugars or other carbon sources may be tested. Alternatively, media may be prepared and the selected bacterium grown as shown by Saarela et al., J. Applied Microbiology. 2005. 99:1330-1339, which is hereby incorporated by reference. Influence of fermentation time, cryoprotectant and neutralization of cell concentrate on freeze-drying survival, storage stability, and acid and bile exposure of the selected bacterium produced without milk-based ingredients.

At large scale, the media is sterilized. Sterilization may be accomplished by Ultra High Temperature (UHT) processing. The UHT processing is performed at very high temperature for short periods of time. The UHT range may be from 135-180° C. For example, the medium may be sterilized from between 10 to 30 seconds at 135° C.

Inoculum can be prepared in flasks or in smaller bioreactors and growth is monitored. For example, the inoculum size may be between approximately 0.5 and 3% of the total bioreactor volume. Depending on the application and need for material, bioreactor volume can be at least 2 L, 10 L, 80 L, 100 L, 250 L, 1000 L, 2500 L, 5000 L, 10,000 L.

Before the inoculation, the bioreactor is prepared with medium at desired pH, temperature, and oxygen concentration. The initial pH of the culture medium may be different that the process set-point. pH stress may be detrimental at low cell centration; the initial pH could be between pH 7.5 and the process set-point. For example, pH may be set between 4.5 and 8.0. During the fermentation, the pH can be controlled through the use of sodium hydroxide, potassium hydroxide, or ammonium hydroxide. The temperature may be controlled from 25° C. to 45° C., for example at 37° C. Anaerobic conditions are created by reducing the level of oxygen in the culture broth from around 8 mg/L to 0 mg/L. For example, nitrogen or gas mixtures (N₂, CO₂, and H₂) may be used in order to establish anaerobic conditions. Alternatively, no gases are used and anaerobic conditions are established by cells consuming remaining oxygen from the medium. Depending on strain and inoculum size, the bioreactor fermentation time can vary. For example, fermentation time can vary from approximately 5 hours to 48 hours.

Reviving bacteria from a frozen state may require special considerations. Production medium may stress cells after a thaw: a specific thaw medium may be required to consistently start a seed train from thawed material. The kinetics of transfer or passage of seed material to fresh medium, for the purposes of increasing the seed volume or maintaining the microbial growth state, may be influenced by the current state of the bacteria (ex. exponential growth, stationary growth, unstressed, stressed).

Inoculation of the production fermenter(s) can impact growth kinetics and cellular activity. The initial state of the bioreactor system must be optimized to facilitate successful and consistent production. The fraction of seed culture to total medium (e.g., a percentage) has a dramatic impact on growth kinetics. The range may be 1-5% of the fermenter's working volume. The initial pH of the culture medium may be different from the process set-point. pH stress may be detrimental at low cell concentration: the initial pH may be between pH 7.5 and the process set-point. Agitation and gas flow into the system during inoculation may be different from the process set-points. Physical and chemical stresses due to both conditions may be detrimental at low cell concentration.

Process conditions and control settings may influence the kinetics of microbial growth and cellular activity. Shifts in process conditions may change membrane composition, production of metabolites, growth rate, cellular stress, etc. Optimal temperature range for growth may vary with strain. The range may be 20-40° C. Optimal pH for cell growth and performance of downstream activity may vary with strain. The range may be pH 5-8. Gasses dissolved in the medium may be used by cells for metabolism. Adjusting concentrations of 02, CO2, and N2 throughout the process may be required. Availability of nutrients may shift cellular growth. Bacteria may have alternate kinetics when excess nutrients are available.

The state of bacteria at the end of a fermentation and during harvesting may impact cell survival and activity. Bacteria may be preconditioned shortly before harvest to better prepare them for the physical and chemical stresses involved in separation and downstream processing. A change in temperature (often reducing to 20-5° C.) may reduce cellular metabolism, slowing growth (and/or death) and physiological change when removed from the fermenter. Effectiveness of centrifugal concentration may be influenced by culture pH. Raising pH by 1-2 points can improve effectiveness of concentration but can also be detrimental to cells. Bacteria may be stressed shortly before harvest by increasing the concentration of salts and/or sugars in the medium. Cells stressed in this way may better survive freezing and lyophilization during downstream.

Separation methods and technology may impact how efficiently bacteria are separated from the culture medium. Solids may be removed using centrifugation techniques. Effectiveness of centrifugal concentration can be influenced by culture pH or by the use of flocculating agents. Raising pH by 1-2 points may improve effectiveness of concentration but can also be detrimental to cells. Bacteria may be stressed shortly before harvest by increasing the concentration of salts and/or sugars in the medium. Cells stressed in this way may better survive freezing and lyophilization during downstream. Additionally, Bacteria may also be separated via filtration. Filtration is superior to centrifugation techniques for purification if the cells require excessive g-minutes to successfully centrifuge. Excipients can be added before after separation. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets mixed with excipients are submerged in liquid nitrogen.

Harvesting can be performed by continuous centrifugation. Product may be resuspended with various excipients to a desired final concentration. Excipients can be added for cryo protection or for protection during lyophilization. Excipients can include, but are not limited to, sucrose, trehalose, or lactose, and these may be alternatively mixed with buffer and anti-oxidants. Prior to lyophilization, droplets of cell pellets mixed with excipients are submerged in liquid nitrogen.

Lyophilization of material, including live bacteria, vesicles, or other bacterial derivative includes a freezing, primary drying, and secondary drying phase. Lyophilization begins with freezing. The product material may or may not be mixed with a lyoprotectant or stabilizer prior to the freezing stage. A product may be frozen prior to the loading of the lyophilizer, or under controlled conditions on the shelf of the lyophilizer. During the next phase, the primary drying phase, ice is removed via sublimation. Here, a vacuum is generated and an appropriate amount of heat is supplied to the material. The ice will sublime while keeping the product temperature below freezing, and below the material's critical temperature (Tc). The temperature of the shelf on which the material is loaded and the chamber vacuum can be manipulated to achieve the desired product temperature. During the secondary drying phase, product-bound water molecules are removed. Here, the temperature is generally raised higher than in the primary drying phase to break any physico-chemical interactions that have formed between the water molecules and the product material. After the freeze-drying process is complete, the chamber may be filled with an inert gas, such as nitrogen. The product may be sealed within the freeze dryer under dry conditions, in a glass vial or other similar container, preventing exposure to atmospheric water and contaminates.

### Example 7: smEV Preparation

Prevotella histicola smEVs were prepared as follows.

smEVs: Downstream processing of smEVs began immediately following harvest of the bioreactor. Centrifugation at 20,000×g was used to remove the cells from the broth. The resulting supernatant was clarified using 0.22 μm filter. The EVs were concentrated and washed using tangential flow filtration (TFF) with flat sheet cassettes ultrafiltration (UF) membranes with 100 kDa molecular weight cutoff (MWCO). Diafiltration (DF) was used to washout small molecules and small proteins using 5 volumes of phosphate buffer solution (PBS). The retentate from TFF was spun down in an ultracentrifuge at 200,000×g for 1 hour to form a pellet rich in EVs called a high-speed pellet (HSP). The pellet was resuspended with minimal PBS and a gradient was prepared with OptiPrep™ density gradient medium and ultracentrifuged at 200,000×g for 16 hours. Of the resulting fractions, 2 middle bands contained EVs. The fractions were washed with 15-fold PBS and the EVs spun down at 200,000×g for 1 hour to create the fractionated HSP or fHSP. It was subsequently resuspended with minimal PBS, pooled, and analyzed for particles per mL and protein content. Dosing was prepared from the particle/mL count to achieve desired concentration. The EVs were characterized using a NanoSight NS300 by Malvern Panalytical in scatter mode using the 532 nm laser.

### Example 8: EV Isolation and Enumeration

The equipment used in EV isolation includes a Sorvall RC-5C centrifuge with SLA-3000 rotor; an Optima XE-90 Ultracentrifuge by Beckman-Coulter 45Ti rotor; a Sorvall wX+Ultra Series Centrifuge by Thermo Scientific; and a Fiberlite F37L-8×100 rotor.

### Bacterial Supernatant Collection and Filtration

Bacteria must be pelleted and filtered away from supernatant in order to recover EVs and not bacteria.

Pellet bacterial culture is generated by using a Sorvall RC-5C centrifuge with the SLA-3000 rotor and centrifuge culture for a minimum of 15 min at a minimum of 7,000 rpm. And then decanting the supernatant into new and sterile container.

The supernatant is filtered through a 0.2 μm filter. For supernatants with poor filterability (less than 300 ml of supernatant pass through filter) a 0.45 μm capsule filter is attached ahead of the 0.2 μm vacuum filter. The filtered supernatant is stored at 4° C. The filtered supernatant can then be concentrated using TFF.

### Isolation of EVs Using Ultracentrifugation

Concentrated supernatant is centrifuged in the ultracentrifuge to pellet EVs and isolate the EVs from smaller biomolecules. The speed is for 200,000×g, time for 1 hour and temperature at 4° C. When rotor has stopped, tubes are removed from the ultracentrifuge and the supernatant is gently poured off. More supernatant is added the tubes are centrifuged again. After all concentrated supernatant has been centrifuged, the pellets generated are referred to as ‘crude’ EV pellets. Sterile 1×PBS is added to pellets, which are placed in a container. The container is placed on a shaker set at speed 70 at 4° C. overnight or longer. The EV pellets are resuspended with additional sterile 1×PBS. The resuspended crude EV samples are stored at 4° C. or at −80° C.

### EV Purification Using Density Gradients

Density gradients are used for EV purification. During ultracentrifugation, particles in the sample will move, and separate, within the graded density medium based on their ‘buoyant’ densities. In this way EVs are separated from other particles, such as sugars, lipids, or other proteins, in the sample.

For EV purification, four different percentages of the density medium (60% Optiprep) are used, a 45% layer, a 35% layer, a 25%, and a 15% layer. This will create the graded layers. A 0% layer is added at the top consisting of sterile 1×PBS. The 45% gradient layer should contain the crude EV sample. 5 ml of sample is added to 15 ml of OptiPrep™. If crude EV sample is less than 5 ml, bring up to volume using sterile 1× PBS.

Using a serological pipette, the 45% gradient mixture is pipetted up and down to mix. The sample is then pipetted into a labeled clean and sterile ultracentrifuge tube. Next, a 10 ml serological pipette is used to slowly add 13 ml of 35% gradient mixture. Next 13 ml of the 25% gradient mixture is added, followed by 13 ml of the 15% mixture and finally 6 ml of sterile 1×PBS. The ultracentrifuge tubes are balanced with sterile 1×PBS. The gradients are carefully placed in a rotor and the ultracentrifuge is set for 200,000×g and 4° C. The gradients are centrifuged for a minimum of 16 hours.

A clean pipette is used to remove fraction(s) of interest, which are added to 15 ml conical tube. These ‘purified’ EV samples are kept at 4° C.

In order to clean and remove residual OptiPrep™ from EVs, 10× volume of PBS are added to purified EVs. The ultracentrifuge is set for 200,000×g and 4° C. Centrifuge and spun for 1 hour. The tubes are carefully removed from ultracentrifuge and the supernatant decanted. The purified EVs are washed until all sample has been pelleted. 1×PBS is added to the purified pellets, which are placed in a container. The container is placed on a shaker set at speed 70 at 4° C. overnight or longer. The ‘purified’ EV pellets are resuspended with additional sterile 1×PBS. The resuspended purified EV samples are stored at 4° C. or at −80° C.

### Example 9: Prevotella smEVs Mechanism of Action

Some bacteria produce extracellular vesicles (smEVs) that share molecular content with the parent bacterium in a particle that is roughly 1/1000 the volume in a non-replicating form.

Preclinical pharmacological effects, mechanism of action, and biodistribution of an orally administered preparation of Prevotella smEVs derived from a single gram-negative bacterial strain of the family Prevotellaceae that was selected from screens of EVs for anti-inflammatory pharmacology. Orally-delivered Prevotella smEVs are gut-restricted bacterial EVs which potently attenuate inflammation in murine models of Th1 and Th17 inflammation.

In this Example, the Prevotella extracellular vesicles (smEVs) used in the studies were isolated from Prevotella Strain B (NRRL accession number B 50329).

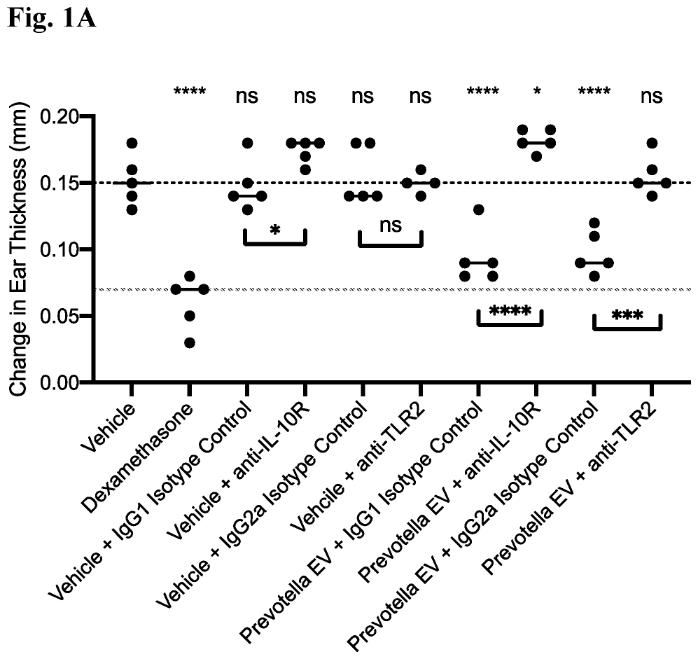

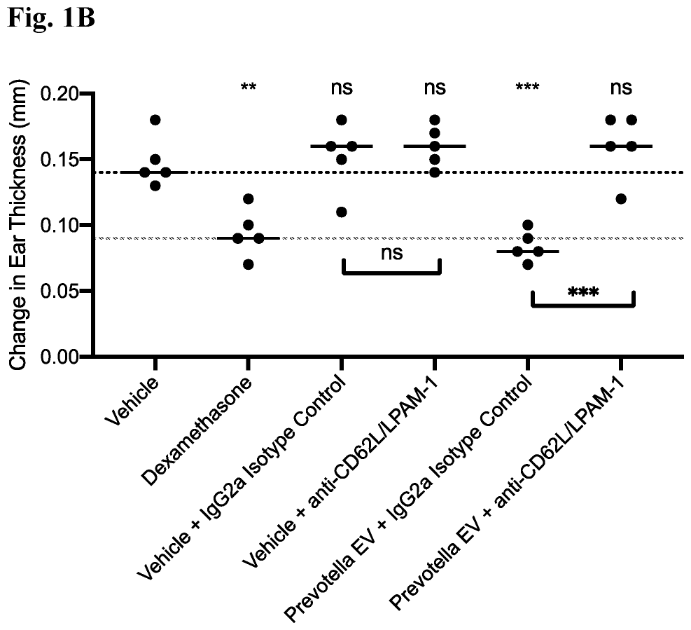

Orally-administered Prevotella smEVs require multiple pathways for anti-inflammatory effects. Mice undergoing a delayed type hypersensitivity (DTH) reaction against keyhole limpet hemagglutinin (KLH) were dosed with 2E10 particles/dose of Prevotella EVs by oral gavage on days 5-8. During the study, various mechanisms of action were interrogated by intraperitoneal injection of antibodies as indicated. FIG. 1A: Graph shows changes in ear thickness 24 hours after challenge with KLH protein and blockade of TLR2 or IL-10R signaling. FIG. 1B: Graph shows changes in ear thickness 24 hours after challenge with KLH protein and inhibition of lymphocyte gut homing (with anti-CD62 and anti-LPAM-1 antibodies). Points indicate individual mice and line shows median change in ear thickness. Data are representative of 2 independent studies. Statistical analyses were performed using a one-way ANOVA (vs. vehicle) or two-tailed unpaired t-test (isotype vs. treated). Ns=not significant, *p<0.05, **p<0.01, ***p<0.001, ****p<0.0001.

Prevotella smEVs are restricted to the gastrointestinal tract. Mice were injected intravenously or orally dosed with 2E10 particles of Prevotella EVs covalently labeled with IRDye800 or dye-only control. After 10 mins, 1 hour, 6 hours, or 24 hours, fluorescence was measured in organs (spleen, liver, gastrointestinal tract, MLNs (mesenteric lymph nodes), kidneys, lungs) using a small animal imaging system (Licor Pearl R). Data not shown. The results showed that orally administered Prevotella EVs appear to be gut restricted.

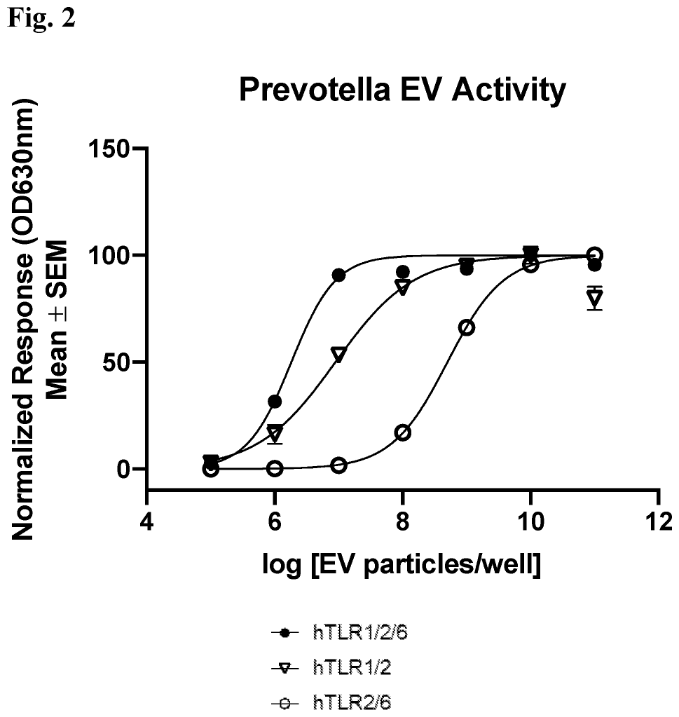

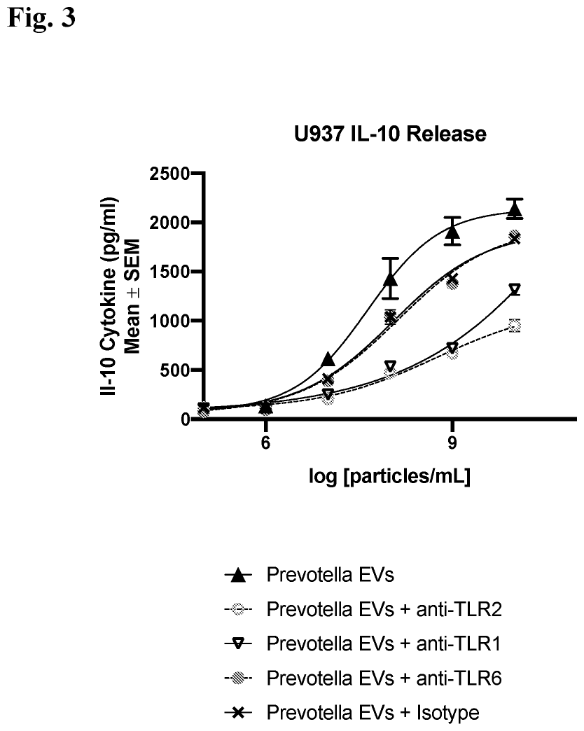

Prevotella smEVs induce release of IL-10 following stimulation of TLR2. FIG. 2: Prevotella EVs stimulate both TLR1/2 and TLR2/6 heterodimers, with greater potency towards the TLR1/2 heterodimer. HEK293-SEAP reporter cells (Invivogen) expressing human TLR1, TLR2, and TLR6 combinations were incubated for 24 hours with Prevotella EVs at the indicated concentrations. Supernatants were collected and analyzed for secreted embryonic alkaline phosphatase (SEAP) production to determine stimulation of TLR2 heterodimers. FIG. 3: Prevotella EV stimulated IL-10 release from U937 cells is impaired by antibody-mediated blockade of either TLR1 or TLR2, but not TLR6. PMA-differentiated human monocytic U937 cells were incubated with Prevotella EVs±2.5 g/mL anti-TLR1, TLR2, TLR6, or isotype control antibody for 24 hours. Supernatants were collected and analyzed for IL-10 response by MSD. Data are representative of 2 independent experiments.

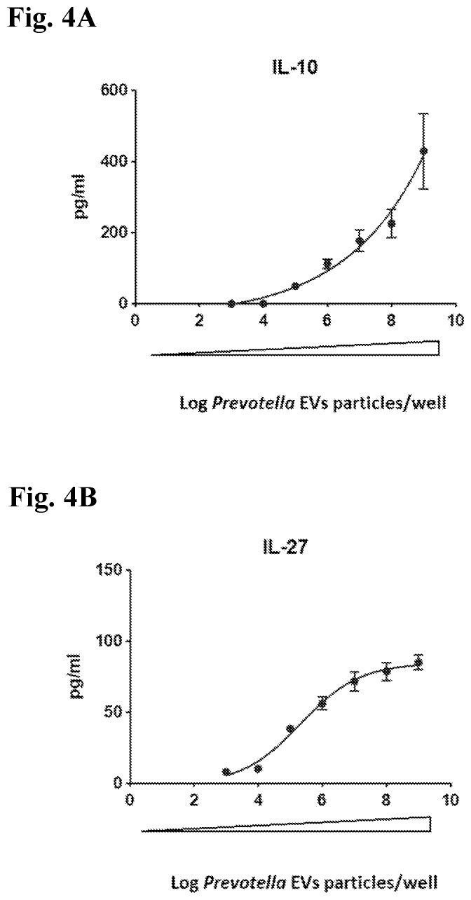

Prevotella smEVs induce IL-10 and IL-27 concentration-dependent production from human PBMCs. PBMCs were isolated from whole blood of six human donors, plated at 100,000 cells per well, rested overnight, and then incubated with varying concentrations of Prevotella EVs for 24 hours. Supernatants were collected and IL-10 (FIG. 4A) and IL-27 (FIG. 4B) concentrations were determined via MSD. Data are representative of 2 independent experiments.

Conclusions: Orally-delivered microbial extracellular vesicles enact broad-based resolution of inflammation establishing homeostatic inflammatory status. The efficacy of Prevotella EVs depends on the stimulation of both the TLR2 receptor and the IL-10 receptor in addition to lymphocyte homing to the intestinal lymphoid tissue. Prevotella EVs induce TLR2-dependent release of IL-10. EVs are an orally-dosed, gut-restricted therapeutic with no apparent safety or tolerability issues in animal models, making for a desirable therapeutic profile.

These data support the development of EVs as a new class of immunotherapeutic drugs. They are particularly effective at engaging the small intestinal axis, acting locally on host cells in the gut to activate distal immune responses. Prevotella EVs are in preclinical development for inflammatory disorders involving both aberrant Th1 and Th17 immune responses.

### Example 10: Prevotella smEVs Lyophilate: DTH Efficacy

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were orally gavaged daily with Prevotella histicola smEVs or dosed intraperitoneally with dexamethasone (positive control) at 1 mg/kg from days 6-8. After dosing on day 8, mice were anaesthetized with isoflurane, left ears were measured for baseline measurements with Fowler calipers and the mice were challenged intradermally with KLH in saline (10 μl) in the left ear and ear thickness measurements were taken at 24 hours.

In this Example, the Prevotella extracellular vesicles (smEVs) used in the studies were isolated from Prevotella Strain B (NRRL accession number B 50329). The smEVs were lyophilized in excipient formula 7a.

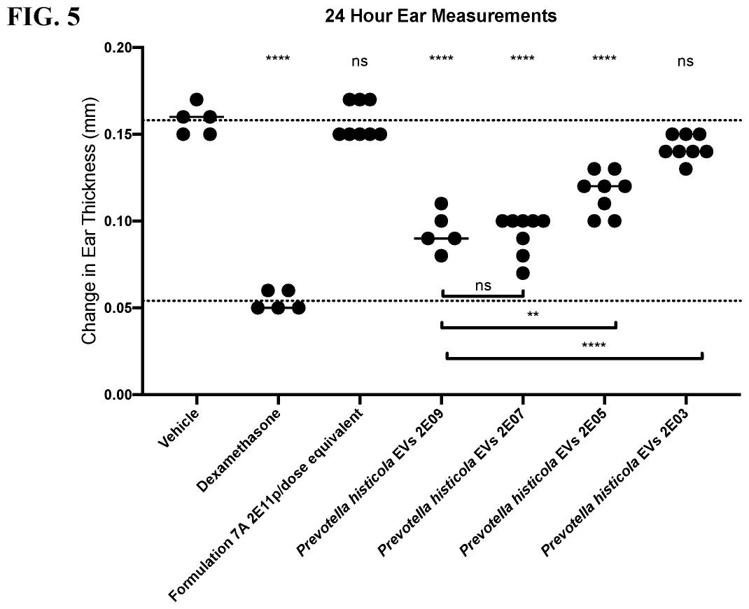

The 24 hour ear measurement results are shown in FIG. 5. EVs made from Prevotella histicola and lyophilized in the excipient of formula 7a were tested in a dose range study with four doses (2E09, 2E07, 2E05, 2E03) for three days of dosing. All doses of the Prevotella histicola EVs were efficacious compared to vehicle except for the lowest dose (2E03) and there was a dose response trend seen. As a negative control, formulation 7a alone was used (with excipient components at a dose equivalent to the amounts present as if 2e11 EVs had been formulated).

### Example 11: Additional Stocks for Drying

EVs from Prevotella histicola bacteria are dried, such as by freeze drying or spray drying using one of the stocks provided in Table K.

TABLE K
Stocks comprising excipients by relative concentration (% w:w).

| For- | |
|---|---|
| mula | Full composition |
| 1 | 40% Sucrose, 15% Trehalose, 20% Mannitol, 25% Dextran 40k |
| 2 | 20% Sucrose, 20% Trehalose, 50% Mannitol, 10% Dextran 40k |
| 3 | 50% Sucrose, 50% Mannitol |
| 4 | 40% Sucrose, 10% Trehalose, 50% Mannitol |
| 5 | 10% Trehalose, 70% Mannitol, 0.5% Sorbitol, 19.5% Dextran 40k |
| 6 | 20% Sucrose, 78% PVP-K30, 1% Citrate, 1% Arginine |
| 7 | 19.5% Trehalose, 80% Mannitol, 0.5% Sorbitol |
| 7a | 20% Trehalose, 80% Mannitol |
| 7e | 27% Sucrose, 20% Trehalose, 53% Mannitol |
| 8 | 10% Trehalose, 75% Mannitol, 15% Dextran 40k |
| 9 | 20% Sucrose, 10% Trehalose, 50% Mannitol, 20% PVP-K30 |
| 10 | 10% Sucrose, 10% Trehalose, 50% Mannitol, 30% B-Cyclodextrin |
| 11 | 95% Mannitol, 5% Poloxamer 188 |
| 12 | 90% Mannitol, 10% Poloxamer 188 |
| 13 | 20% Sucrose, 10% Trehalose, 50% Mannitol, 20% Ficoll |
| 14 | 20% Sucrose, 78% Ficoll, 1% Citrate, 1% Arginine |
| 15 | 19.5% Trehalose, 70% Mannitol, 0.5% Sorbitol, 10% Dextran 40k |
| 16 | 19.5% Trehalose, 75% Mannitol, 0.5% Sorbitol, 5% Dextran 40k |
| 17 | 20% Trehalose, 80% Mannitol |
| 18 | 10% Trehalose, 60% Mannitol, 30% Dextran 40k |
| 19 | 10% Trehalose, 30% Dextran 40k, 60% Maltodextrin |
| 20 | 100% Mannitol |
| 21 | 20% Mannitol, 60% PEG 6000, 20% Trehalose |
| 22 | 10% Mannitol, 60% PEG 6000, 30% Trehalose |
| 23 | 70% PEG 6000, 30% Trehalose |
| 24 | 70% PEG 6000, 30% Mannitol |

The drying conditions in Table L are used for lyophilization.

TABLE L
General conservative lyophilization cycle for EVs.

| | Ramp time | Hold time | Shelf temp | Vacuum |
|---|---|---|---|---|
| Step | (min) | (min) | (C.) | (mTorr) |
| Freezing | 200 | 360 | −45 | 100-300 |
| Primary drying | 75 | 5000 | −20 | 100-300 |
| Secondary | 180 | 1000 | 25 | 100-300 |
| drying | | | | |
| Hold | 0 | N/A | 25 | 100-300 |

### Example 12: Spray-Dried Powders of Prevotella histicola smEVs

In this Example, the extracellular vesicles (smEVs) used in the studies were isolated from Prevotella histicola Strain B.

The smEVs were spray dried as follows:

EV retentate was mixed with one of the excipients provided in Table P.

TABLE P
Stocks comprising excipients by relative concentration (% w:w)

| Formula | Full composition |
|---|---|
| 7a | 80% Mannitol, 20% Trehalose |
| 25 | 100% Trehalose |
| 26 | Maltodextrin-Trehalose (20:60:20) |
| 27 | Maltodextrin-Trehalose (70:30) |
| 28 | PEG6000-Trehalose (70:30) |
| 29 | Mannitol-Maltodextrin-Trehalose (20:60:20) |

The spray drying was performed at 100° C. or 130° C. Temperatures are also included in Table Q.

Post spray drying, each powder was analyzed for moisture content (MC) (by Karl Fischer titration (KF)) and particles (particles/mg spray-dried powder (p/mg)) (quantification by nanoparticle tracking analysis (NTA) using Zetaview). Results are shown in Table Q. EXP7A is stock of formula 7a.

TABLE Q
Moisture content and particle content of spray dried samples

| | Inlet | Moisture | |
|---|---|---|---|
| Sample ID | temperature (° C.) | Content (%) | Particles/mg |
| EXP7A-130° C. inlet | 130 | 3.64 | 1.30E+10 |
| EXP7A-100° C. inlet | 100 | 2.54 | 1.25E+10 |
| Man-Malt-Tre | 130 | 5.35 | 1.40E+10 |
| (20:60:20) | | | |
| Malt-Tre (70:30) | 130 | 8.38 | 2.00E+10 |
| 100% Trehalose | 130 | 6.37 | 1.60E+10 |

Man: mannitol; Malt: maltodextrin; Tre: trehalose.

Spray drying was also performed using a stock that consisted of PEG6000-Mannitol-Trehalose (60:20:20). However, less dried product was recovered relative to other methods and was not analyzed further.

Prevotella histicola smEVs were spray dried or lyophilized in stock of formulation 7a (F7A) at two concentrations: 25× and 500×, with an inlet temperature of 130° C.

The comparison of particles/mg spray-dried powder and size are shown in Table R. SD=spray dried; L0.47=lyophilized; 0.47 refers to the stock ratio used: 47 g of excipient with every 100 g of retentate.

The particle packing and size of the spray dried and lyophilized EVs were similar with both methods of drying.

TABLE R

| Sample | Particles/mg | CV | Size (nm) |
|---|---|---|---|
| 25X L0.47 F7A | 3.35e+9 | 2.10% | 172.7 |
| 25X SD F7A | 3.95e+9 | 0.00% | 164.8 |
| 500X L0.47 F7A | 5.50e+10 | 0.00% | 165.1 |
| 500X SD F7A | 4.88e+10 | 0.70% | 163.8 |

### Example 13: Prevotella histicola smEVs and Anti-TNFa Antibody: DTH Efficacy

In this Example, the extracellular vesicles (smEVs) used in the studies were isolated from Prevotella histicola Strain B.

Female 5 week old C57BL/6 mice were purchased from Taconic Biosciences and acclimated at a vivarium for one week. Mice were primed with an emulsion of KLH and CFA (1:1) by subcutaneous immunization on day 0. Mice were injected intraperitoneally on day 0, 3 and 6 with 3 mg/kg of anti-TNF alpha (Anti-TNFa) antibody (Clone: XT3.11 purchased from BioXCell) or equivalent isotype control (IgG1 also purchased from BioXCell). Mice were orally gavaged daily with Prevotella histicola extracellular vesicles (smEVs) or dosed intraperitoneally with dexamethasone at 1 mg/kg from days 5-8. After dosing on day 8, mice were anaesthetized with isoflurane, left ears were measured for baseline measurements with Fowler calipers and the mice were challenged intradermally with KLH in saline (10 μl) in the left ear. Ear thickness measurements were taken at 24 hours.

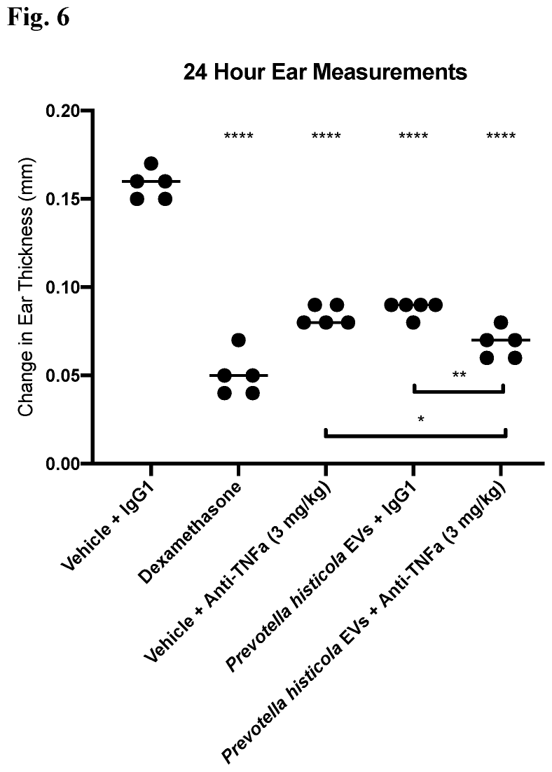

The 24 hour ear measurement results are shown in FIG. 6. smEVs made from Prevotella histicola (at 2E09 particles per dose) were tested alone or in combination with 3 mg/kg anti-TNF alpha antibody or equivalent isotype control (IgG1). Three mg/kg anti-TNF alpha antibody were also tested alone or with vehicle control. The combination of Prevotella histicola smEVs with anti-TNF alpha was more efficacious than both Prevotella histicola smEVs alone and anti-TNF alpha antibody alone.

### Example 14: Oral Dosing of Prevotella histicola smEVs does not Inhibit the Immune System Under Non-Inflammatory Conditions

In this Example, the extracellular vesicles (smEVs) used in the studies were isolated from Prevotella histicola Strain B.

Oral dosing prior to or following KLH DTH response: Two groups of mice were used in this study: ‘pre-immunization’ mice which were dosed with smEVs or controls prior to immunization, and ‘post-immunization’ mice, which were dosed with smEVs or controls after immunization. Starting on day −5, ‘pre-immunization’ mice were dosed daily for 4 days with PBS vehicle PO, dexamethasone (1 mg/kg, IP (intraperitoneally)), or Prevotella smEVs or smEVs from another bacterial strain PO (orally). On day 0, all mice were immunized by subcutaneous injection with KLH emulsified with Complete Freund's Adjuvant (CFA). ‘Post-immunization’ mice were dosed daily starting on day 5 after immunization with sucrose vehicle, Prevotella smEVs or smEVs from another bacterial strain PO, or dexamethasone (1 mg/kg, IP) for 4 days. On day 8, baseline ear thickness was measured using calipers, then mice were challenged by intradermal ear injection with KLH. After 24 hours, the change in ear thickness was evaluated and compared to baseline measurements. Prevotella smEVs and smEVs from another bacterial strain were used at 2E9 particles/dose. The other bacterial strain smEVs were selected for study based on for their ability to decrease inflammation in a DTH model.

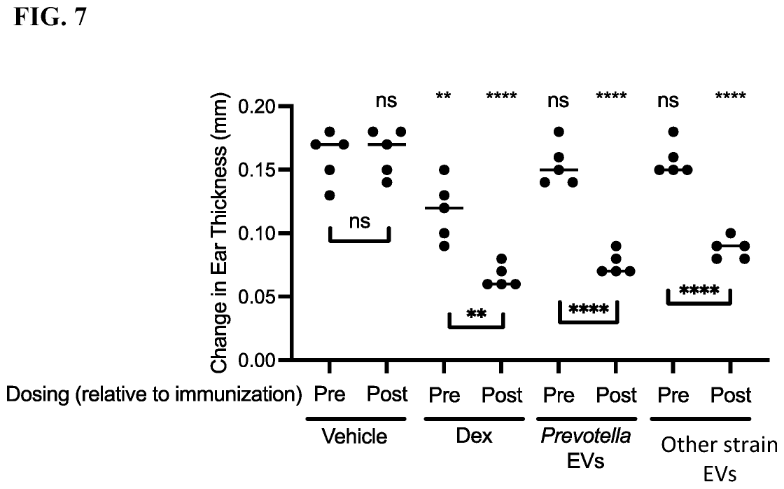

RESULTS AND CONCLUSION: The results are shown in FIG. 7. Treatment with dexamethasone reduced inflammation in a subsequent DTH response. Dosing with Prevotella smEVs or smEVs from another bacterial strain prior to immunization did not reduce inflammation that was subsequently induced by the intradermal ear challenge. Dosing with Prevotella smEVs or smEVs from another bacterial strain after immunization resulted in reduction of ear inflammation after the challenge. These data show that smEVs dosed in the absence of inflammation do not inhibit the immune response, but instead, resolve an ongoing inflammatory response.

### Example 15: Antigen-Specific Response During Induction of Inflammation are not Required for Resolution of Inflammation in a Subsequent KLH DTH Response by Prevotella sm EVs

In this Example, the extracellular vesicles (smEVs) used in the studies were isolated from Prevotella histicola Strain B.

Complete Freund's adjuvant (CFA) (which contains TLR agonists and heat-killed Mycobacterium tuberculosis) and Incomplete Freud's adjuvant (IFA) (which contains only TLR agonists) have been shown to drive Th1 and Th2 immune responses, respectively. Moreover. IFA-PBS immunization induces an inflammatory response that is absent of any antigen.

Dosing following induction of inflammation is sufficient to induce inflammatory resolution in a subsequent KLH DTH challenge: On day −9. PBS-IFA′ and PBS-CFA′ mice were immunized by subcutaneous injection with PBS emulsified with Complete or Incomplete Freund's Adjuvant (CFA or IFA, respectively). On Day −5, mice were dosed daily for 4 days with PBS vehicle PO or Prevotella smEVs PO. On day 0, mice were immunized with KLH emulsified with Complete Freund's Adjuvant but no smEVs were administered after KLH-CFA immunization. On day 5, baseline ear thickness was measured using calipers, then mice were challenged by intradermal ear injection with KLH. After 24 hours, the change in ear thickness was evaluated and compared to baseline measurements. Prevotella smEVs were used at 2E9 particles/dose.

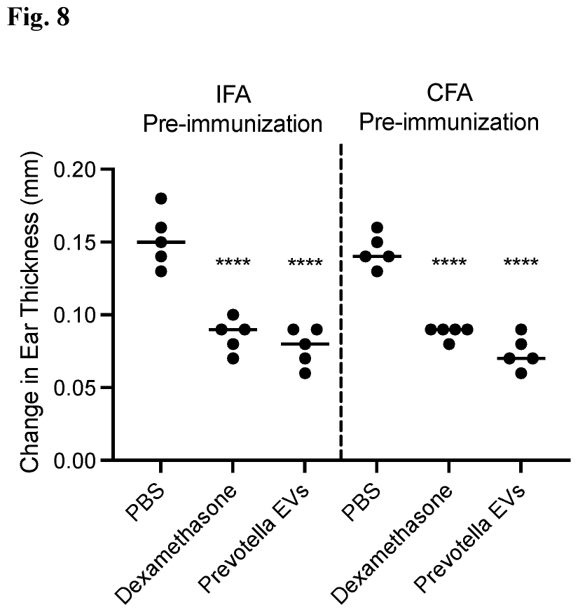

RESULTS AND CONCLUSION: The results are shown in FIG. 8. smEVs that were dosed only during induction of inflammation using CFA or IFA, prior to KLH sensitization, were able to inhibit the KLH DTH response after ear challenge. These data show that the induction of generalized inflammation in the absence of the relevant antigen used in the DTH (KLH) is sufficient for efficacy in the KLH DTH, and is therefore an antigen-independent mechanism that can reduce antigen-specific inflammation.

### Example 16: Adoptive Transfer of CD4+ T Cells from KLH-CFA Immunized Mice Dosed with Prevotella smEVs Confers Immune Resolution in Recipient KLH-CFA Immunized Mice

In this Example, the extracellular vesicles (smEVs) used in the studies were isolated from Prevotella histicola Strain B.

Adoptive cell transfer of CD4+ T cells from orally dosed KLH-CFA immunized mice into KLH-DTH immunized recipient mice: On day 0, ‘donor mice’ were immunized by subcutaneous injection with KLH emulsified with Complete Freund's Adjuvant. On day 5, ‘recipient mice’ were immunized by subcutaneous injection with KLH emulsified with Complete Freund's Adjuvant and ‘donor mice’ were dosed daily for 4 days with PBS vehicle or Prevotella smEVs or smEVs from another bacterial strain PO. On day 9, brachial, axillary, and inguinal lymph nodes and spleens were harvested from donor mice and CD4+ T cells were enriched by negative selection on magnetic beads. Enriched cells were then counted, washed with PBS (300×g, 10 mins, 4° C.), and resuspended at 5×10⁷ cells/mL in PBS. After enrichment, 5×10⁶ CD4+ T cells were transferred into ‘recipient mice’ by IP injection. On day 12, baseline ear thickness was measured using calipers, then recipient mice were challenged by intradermal ear injection with KLH. After 24 hours, the change in ear thickness was evaluated and compared to baseline measurements. Prevotella smEVs and smEVs from another bacterial strain were used at 2E9 particles/dose. The other bacterial strain smEVs were selected for study based on for their ability to decrease inflammation in a DTH model.

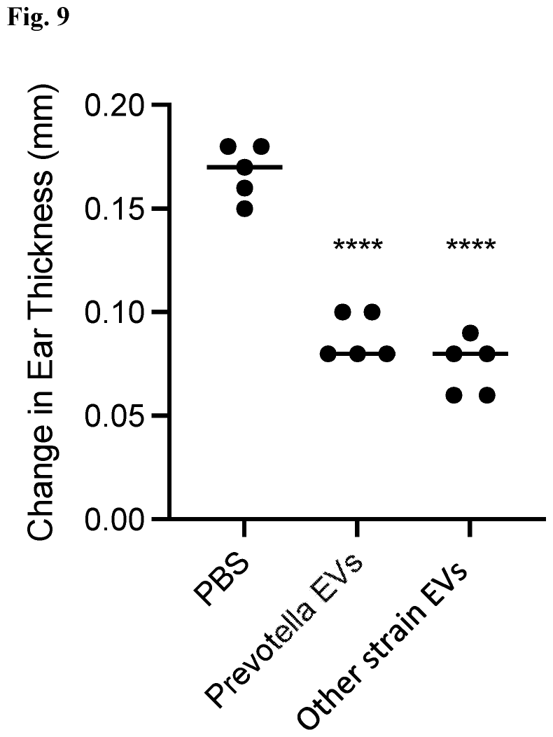

RESULTS AND CONCLUSION: The results are shown in FIG. 9. CD4+ T cells transferred from mice treated with Prevotella and smEVs from another bacterial strain were able to inhibit the DTH response in recipient mice that had not been treated with smEVs, whereas CD4+ T cells from PBS vehicle-treated donors were not efficacious in recipient mice. These data demonstrate that oral dosing with Prevotella smEVs or smEVs from another bacterial strain generated a population of CD4+ T cells that can resolve inflammation in inflamed mice that had not received oral dosing of Prevotella smEVs or smEVs from another bacterial strain.

### Example 17: During Oral Dosing of Prevotella smEVs, TLR2 Signaling is Essential for the Generation of CD4+ T Cells that can be Adoptively Transferred to Mediate Immune Resolution in a Recipient KLH DTH Model

In this Example, the extracellular vesicles (smEVs) used in the studies were isolated from Prevotella histicola Strain B.

The small intestine contains a plethora of Toll-like Receptors (TLR) that recognize multiple components of microbes. We have previously shown that oral dosing of Prevotella smEVs can generate a population of CD4+ T cells that mediate immune resolution following adoptive transfer into a KLH DTH model.

Adoptive cell transfer of CD4+ T cells from orally-dosed KLH-CFA immunized mice treated with anti-TLR2 into KLH-DTH immunized recipient mice: On day 0, ‘donor mice’ were immunized by subcutaneous injection with KLH emulsified with Complete Freund's Adjuvant and treated with anti-TLR2 or Rat IgG2a isotype control antibodies (2 μg/mouse) IP on days 0, 3, and 6. On day 5, ‘recipient mice’ were immunized by subcutaneous injection with KLH emulsified with Complete Freund's Adjuvant and ‘donor mice’ were dosed daily for 4 days with PBS vehicle or Prevotella smEVs PO. On day 9, brachial, axillary, and inguinal lymph nodes and spleens were harvested from donor mice and CD4+ T cells were enriched by negative selection on magnetic beads. Enriched cells were then counted, washed with PBS (300×g, 10 mins, 4° C.), and resuspended at 5×10⁷ cells/mL in PBS. After enrichment, 5×10⁶ CD4+ T cells were transferred into ‘recipient mice’ by IP injection. On day 12, baseline ear thickness was measured using calipers, then recipient mice were challenged by intradermal ear injection with KLH. After 24 hours, the change in ear thickness was evaluated and compared to baseline measurements. Prevotella smEVs were used at 2E9 particles/dose.

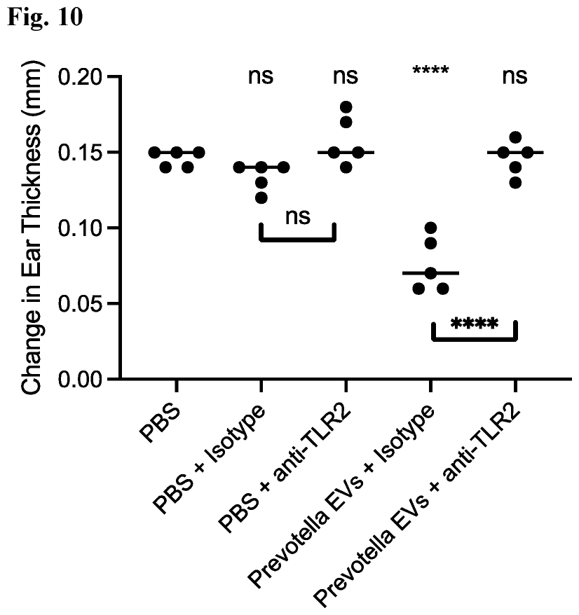

RESULTS AND CONCLUSION: The results are shown in FIG. 10. CD4+ T cells isolated from mice treated with PBS vehicle and an anti-TLR2 blocking antibody did not inhibit the DTH response to KLH compared to CD4+ T cells from mice treated with PBS alone, or PBS with an isotype control antibody. CD4+ T cells from mice treated with Prevotella smEVs and an isotype control antibody were efficacious in the model and able to inhibit ear inflammation after the intradermal KLH challenge, as previously shown. However, CD4+ T cells transferred from mice that were dosed with Prevotella smEVs in the presence of anti-TLR2 antibody were not efficacious in recipient mice, and ear swelling was comparable to that of PBS-treated CD4+ T cells. These data demonstrate that TLR2 signaling was essential for the Prevotella EV-induced generation of immune resolving CD4+ T cells as antibody-mediated blockade of this receptor in donor mice abrogated the efficacy of CD4+ T cells transferred from Prevotella EV-dosed mice.

### Example 18: Adoptive Transfer of CD8+ T Cells from KLH-CFA Immunized Mice Dosed with Prevotella smEVs does not Confer Immune Resolution in Recipient KLH-CFA Immunized Mice

In this Example, the extracellular vesicles (smEVs) used in the studies were isolated from Prevotella histicola Strain B.

We have previously shown that the adoptive transfer of CD4+ T cells from Prevotella EV-dosed mice have the capacity to resolve inflammation in KLH-DTH recipients.

Adoptive cell transfer of CD8+ T cells from orally dosed KLH-CFA immunized mice into KLH-DTH immunized recipient mice: On day 0, ‘donor mice were immunized by subcutaneous injection with KLH emulsified with Complete Freund's Adjuvant. On day 5, ‘recipient mice’ were immunized by subcutaneous injection with KLH emulsified with Complete Freund's Adjuvant and ‘donor mice’ were dosed QD for 4 days with PBS or Prevotella smEVs or smEVs from another bacterial strain PO. On day 9, brachial, axillary, and inguinal lymph nodes and spleens were harvested from donor mice and CD4+ T cells were enriched by negative selection on magnetic beads. Enriched cells were then counted, washed with PBS (300×g, 10 mins, 4° C.), and resuspended at 5×10⁷ cells/mL in PBS. After enrichment, 5×10⁶ CD8+ T cells were transferred into ‘recipient mice’ by IP injection. On day 12, baseline ear thickness was measured using calipers, then recipient mice were challenged by intradermal ear injection with KLH. After 24 hours, the change in ear thickness was evaluated and compared to baseline measurements. Prevotella smEVs and smEVs from another bacterial strain were used at 2E9 particles/dose. The other bacterial strain smEVs were selected for study based on for their ability to decrease inflammation in a DTH model.

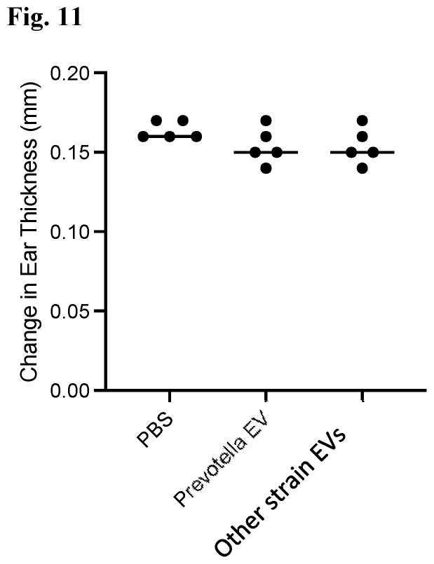

RESULTS AND CONCLUSION: The results are shown in FIG. 11. Unlike with CD4+ T cells, the adoptive cell transfer of CD8+ T cells from KLH-CFA inflamed Prevotella smEV—and smEVs from another bacterial strain—dosed donor mice into KLH-CFA inflamed recipient mice did not confer immune resolution of a KLH DTH model in the recipient mice. These data suggest that oral dosing of Prevotella smEVs and smEVs from another bacterial strain induce a population of immune-resolving CD4+ T cells, but not CD8+ T cells, that can inhibit the DTH response in recipient mice.

### Example 19: Preparation of a Solid Dosage Form Comprising Prevotella histicola smEVs

Tablets of the Recipes in Table S were Prepared:

TABLE S
Compositions of active tablets (400 mg)

| | Low | Medium | High |
|---|---|---|---|
| Tablets | Strength | Strength | Strength |
| Prevotella histicola smEVs | 10% | 10% | 62.5% |
| (drug substance/powder) | (LS DS) | (HS DS) | (HS DS) |
| SMCC HD90 | 72.5% | 72.5% | 20.0% |
| Crospovidone | 15.0% | 15.0% | 15.0% |
| SiO₂ | 1.0% | 1.0% | 1.0% |
| Magnesium stearate | 1.5% | 1.5% | 1.5% |
| Tablet weight (mg) | 400 | 400 | 400 |

The Prevotella histicola smEVs in Table S are from strain Prevotella histicola Strain B 50329 (NRRL accession number B 50329).

Drug substance (powder) was prepared by lyophilization using excipient formula 7a. HS DS: high strength drug substance. LS DS: low strength drug substance. LS DS was prepared by diluting HS DS 10× (using lyophilzation excipients) before lyophilization.

To prepare the pharmaceutical composition tablets, wet granulation was performed on the drug substance (pharmaceutical agent) containing the smEVs. Drug substance was (i) mixed with water; (ii) dried on a fluid bed dryer; (iii) milled; (iv) then blended with the drug product excipients provided in Table S.

The tablets were 5.5 mm×15.8 mm.

### Example 20: Preparation of a Solid Dosage Form Comprising Prevotella histicola smEVs

Capsule of the Recipes in Table T were Prepared:

TABLE T
Compositions of active capsules

| | Low | Medium | High |
|---|---|---|---|
| Capsules | Strength | Strength | Strength |
| Prevotella histicola smEVs | 10% | 10% | 90% |
| (drug substance/powder) | (LS DS) | (HS DS) | (HS DS) |
| Mannitol | 87.5% | 87.5% | 7.5% |
| SiO2 | 1.0% | 1.0% | 1.0% |
| Magnesium stearate | 1.5% | 1.5% | 1.5% |
| Capsule filled weight (mg) | 360 | 360 | 360 |

The Prevotella histicola smEVs in Table T are from strain Prevotella histicola Strain B 50329 (NRRL accession number B 50329).

Drug substance (powder) was prepared by lyophilization using excipient formula 7a. HS DS: high strength drug substance. LS DS: low strength drug substance. LS DS was prepared by diluting HS DS 10× (using lyophilzation excipients) before lyophilization. HS DS: high strength drug substance. LS DS: low strength drug substance.

LS DS was prepared by diluting HS DS 10× (using lyophilization excipients) before lyophilization.

To prepare the pharmaceutical composition capsules, wet granulation was performed on the drug substance (pharmaceutical agent) containing the smEVs. Drug substance was (i) mixed with water; (ii) dried on a fluid bed dryer; (iii) milled; (iv) then blended with the drug product excipients provided in Table T.

The capsules were size 0.

### Example 21: Testing of Drug Substance

The Prevotella histicola smEVs used to prepare drug substance (DS) are from strain Prevotella histicola Strain B 50329 (NRRL accession number B 50329).

Drug substance (powder) was prepared by lyophilization using excipient formula 7a (80% mannitol: 20% trehalose). HS DS: high strength drug substance. LS DS: low strength drug substance. LS DS was prepared by diluting HS DS 10× (using lyophilzation excipients) before lyophilization.

HS DS and LS DS were assessed in in vitro assays performed in human peripheral blood mononuclear cells (PBMCs), macrophages and dendritic cells. Cells were obtained from five donors: the five donors were run as biological replicates and data were expressed as average response. Serial dilutions were performed: 1×10³, 1×10⁴, 1×10⁵, 1×10⁶, 1×10⁷, 1×10⁸, 1×10⁹, and 1×10¹⁰ particles of both HS DS and LS DS were evaluated for their effects on cytokine secretion from the PBMCs, macrophages and dendritic cells. Similar trends were seen across all three cell populations. HS DS and LS DS were able to induce IL-10, IL-27, IL-6, IP-10, and TNFa from all three cell populations. Dendritic cells produced low levels of IL-27 and IP-10 in response to both DS.

HS DS and LS DS were assessed in in vitro assays performed in U937. Serial dilutions were performed: 7.06×10³, 2.12×10⁴, 6.35×10⁴, 1.91×10⁵, 5.72×10⁵, 1.71×10⁶, 5.14×10⁶, 1.54×10⁷, 4.63×10⁷, 1.39×10⁸, 4.17×10⁸, and 1.25×10⁹ particles of both HS DS and LS DS were evaluated for their effects on cytokine secretion from the U937 cells. HS DS and LS DS were able to induce IL-10, IL-27, IL-6, IP-10, and TNFa from the U937 cells.

### INCORPORATION BY REFERENCE

All publications patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of activating TLR2 in a subject comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

2. A method of activating an anti-inflammatory cytokine response in a subject comprising administering (e.g., orally administering) to the subject a dose (e.g., a therapeutically effective dose) of extracellular vesicles (EVs) from a Prevotella histicola strain and/or a composition (e.g., a solution, dried form and/or therapeutic composition) comprising the extracellular vesicles.

3. The method of claim 1 or 2, wherein the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329).

4. The method of any one of claims 1 to 3, wherein the Prevotella histicola strain is the Prevotella histicola Strain B (NRRL accession number B 50329).

5. The method of any one of claims 1 to 4, wherein the subject has an immune disorder.

6. The method of claim 5, wherein the immune disorder is arthrosclerosis, arthritis, phlebitis, vasculitis, and lymphangitis. cholangitis, cholecystitis, enteritis, enterocolitis, gastritis, gastroenteritis, inflammatory bowel disease, ileitis, proctitis, Crohn's disease, ulcerative colitis, irritable bowel syndrome, microscopic colitis, lymphocytic-plasmocytic enteritis, coeliac disease, collagenous colitis, lymphocytic colitis, eosinophilic enterocolitis, indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, primary sclerosing cholangitis, cervicitis, chorioamnionitis, endometritis, epididymitis, omphalitis, oophoritis, orchitis, salpingitis, tubo-ovarian abscess, urethritis, vaginitis, vulvitis, vulvodynia, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, diabetes mellitus type 1, giant cell arteritis, good pasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, Ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, contact hypersensitivity, contact dermatitis (including that due to poison ivy), urticaria, skin allergies, respiratory allergies (hay fever, allergic rhinitis, house dust mite allergy), appendicitis, dermatitis, dermatomyositis, endocarditis, fibrositis, gingivitis, glossitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, stomatitis, transplant rejection, acute pancreatitis, chronic pancreatitis, acute respiratory distress syndrome, Sexary's syndrome, congenital adrenal hyperplasia, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenia purpura in adults, secondary thrombocytopenia in adults, acquired (autoimmune) haemolytic anemia, leukemia and lymphomas in adults, acute leukemia of childhood, regional enteritis, autoimmune vasculitis, chronic obstructive pulmonary disease, or sepsis.

7. The method of claim 6, wherein the subject has psoriasis.

8. The method of claim 6, wherein the subject has atopic dermatitis.

9. The method of any one of claims 1 to 4, wherein the subject has a Th1 mediated inflammatory disease.

10. The method of any one of claims 1 to 4, wherein the subject has a Th2 mediated inflammatory disease.

11. The method of any one of claims 1 to 4, wherein the subject has a Th17 mediated inflammatory disease.

12. The method of any one of claims 1 to 11, wherein the EVs are administered orally.

13. The method of any one of claims 1 to 12, wherein the dose is in the form of one or more capsules, optionally comprising an enteric-coating (e.g., enteric-coated capsules).

14. The method of any one of claims 1 to 12, wherein the dose is in the form of one or more tablets, optionally comprising an enteric-coating (e.g., enteric-coated tablets).

15. The method of any one of claims 1 to 12, wherein the dose is in the form of one or more mini-tablets.

16. The method of claim 15, wherein the mini-tablets are enteric-coated mini-tablets.

17. The method of any one of claims 1 to 12, wherein the dose is in the form of a non-enteric coated capsule comprising an enteric-coated mini-tablet.

18. The method of any one of claims 1 to 17, wherein the dose is administered in combination with an additional therapeutic agent.

19. A solution comprising extracellular vesicles (EVs) from a Prevotella histicola strain and an excipient that comprises a bulking agent.

20. A therapeutic composition comprising the solution of claim 19, wherein the composition further comprises a pharmaceutically acceptable excipient.

21. The therapeutic composition of claim 20, wherein the pharmaceutically acceptable excipient comprises a glidant, lubricant, and/or diluent.

22. A dried form comprising extracellular vesicles (EVs) from a Prevotella histicola strain and an excipient that comprises a bulking agent.

23. A therapeutic composition comprising the dried form of claim 22, wherein the composition further comprises a pharmaceutically acceptable excipient.

24. The therapeutic composition of claim 23, wherein the pharmaceutically acceptable excipient comprises a glidant, lubricant, and/or diluent.

25. A therapeutic composition comprising extracellular vesicles (EVs) from a Prevotella histicola strain and an excipient that comprises a bulking agent.

26. A solution comprising extracellular vesicles (EVs) from a Prevotella histicola strain and excipients of a stock comprising one or more excipients, wherein the stock comprises of a formula provided in Table A, B, C, D, K, or P.

27. A therapeutic composition comprising the solution of claim 26, wherein the composition further comprises a pharmaceutically acceptable excipient.

28. The therapeutic composition of claim 27, wherein the pharmaceutically acceptable excipient comprises a glidant, lubricant, and/or diluent.

29. A dried form comprising extracellular vesicles (EVs) from a Prevotella histicola strain and excipients of a stock comprising one or more excipients, wherein the stock comprises a formula provided in Table A, B, C, D, K, or P.

30. A therapeutic composition comprising the dried form of claim 29, wherein the composition further comprises a pharmaceutically acceptable excipient.

31. The therapeutic composition of claim 30, wherein the pharmaceutically acceptable excipient comprises a glidant, lubricant, and/or diluent.

32. A method of treating a subject (e.g., a subject in need of treatment), the method comprising:
administering to the subject the solution, dried form, or therapeutic composition (e.g., a therapeutically effective amount thereof) of any one of claims 19 to 31.

33. The solution, powder, or therapeutic composition (e.g., a therapeutically effective amount thereof) of any one of claims 19 to 31 for use in treating a subject (e.g., a subject in need of treatment).

34. Use of the solution, dried form, or therapeutic composition (e.g., a therapeutically effective amount thereof) of any one of claims 19 to 31 for the preparation of a medicament for treating a subject (e.g., human) (e.g., a subject in need of treatment).

35. The method/solution/powder/therapeutic composition/use of any one of claims 32 to 34, wherein the solution/powder/therapeutic composition is orally administered (e.g., is for oral administration).

36. The method/solution/powder/therapeutic composition/use of any one of claims 32 to 35, wherein the subject has an immune disorder.

37. The method/solution/powder/therapeutic composition/use of claim 36, wherein the immune disorder is arthrosclerosis, arthritis, phlebitis, vasculitis, and lymphangitis. cholangitis, cholecystitis, enteritis, enterocolitis, gastritis, gastroenteritis, inflammatory bowel disease, ileitis, proctitis, Crohn's disease, ulcerative colitis, irritable bowel syndrome, microscopic colitis, lymphocytic-plasmocytic enteritis, coeliac disease, collagenous colitis, lymphocytic colitis, eosinophilic enterocolitis, indeterminate colitis, pseudomembranous colitis (necrotizing colitis), ischemic inflammatory bowel disease, Behcet's disease, sarcoidosis, scleroderma, IBD-associated dysplasia, dysplasia associated masses or lesions, primary sclerosing cholangitis, cervicitis, chorioamnionitis, endometritis, epididymitis, omphalitis, oophoritis, orchitis, salpingitis, tubo-ovarian abscess, urethritis, vaginitis, vulvitis, vulvodynia, acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, diabetes mellitus type 1, giant cell arteritis, good pasture's syndrome, Grave's disease, Guillain-Barre syndrome, Hashimoto's disease, Henoch-Schonlein purpura, Kawasaki's disease, lupus erythematosus, microscopic colitis, microscopic polyarteritis, mixed connective tissue disease, Muckle-Wells syndrome, multiple sclerosis, myasthenia gravis, opsoclonus myoclonus syndrome, optic neuritis, Ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, Lyme disease, morphea, psoriasis, sarcoidosis, scleroderma, contact hypersensitivity, contact dermatitis (including that due to poison ivy), urticaria, skin allergies, respiratory allergies (hay fever, allergic rhinitis, house dust mite allergy), appendicitis, dermatitis, dermatomyositis, endocarditis, fibrositis, gingivitis, glossitis, hidradenitis suppurativa, iritis, laryngitis, mastitis, myocarditis, nephritis, otitis, pancreatitis, parotitis, pericarditis, peritonitis, pharyngitis, pleuritis, pneumonitis, prostatitis, pyelonephritis, stomatitis, transplant rejection, acute pancreatitis, chronic pancreatitis, acute respiratory distress syndrome, Sexary's syndrome, congenital adrenal hyperplasia, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, atopic dermatitis, drug hypersensitivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenia purpura in adults, secondary thrombocytopenia in adults, acquired (autoimmune) haemolytic anemia, leukemia and lymphomas in adults, acute leukemia of childhood, regional enteritis, autoimmune vasculitis, chronic obstructive pulmonary disease, or sepsis.

38. The method/solution/powder/therapeutic composition/use of any one of claims 32 to 37, wherein the subject has psoriasis.

39. The method/solution/powder/therapeutic composition/use of any one of claims 32 to 37, wherein the subject has atopic dermatitis.

40. The method/solution/powder/therapeutic composition/use of any one of claims 32 to 35, wherein the subject has a Th1 mediated inflammatory disease.

41. The method/solution/powder/therapeutic composition/use of any one of claims 32 to 35, wherein the subject has a Th2 mediated inflammatory disease.

42. The method/solution/powder/therapeutic composition/use of any one of claims 32 to 35, wherein the subject has a Th17 mediated inflammatory disease.

43. The method/solution/dried form/therapeutic composition/use of any one of claims 32 to 42, wherein the solution/powder/therapeutic composition is administered in combination with an additional therapeutic agent.

44. The method/solution/dried form/therapeutic composition/use of any one of claims 19 to 43, wherein the extracellular vesicles are from a Prevotella histicola strain comprising at least 95%, at least 96%, at least 97%, at least 98%, or at least 99% sequence identity (e.g., at least 99.5% sequence identity, at least 99.6% sequence identity, at least 99.7% sequence identity, at least 99.8% sequence identity, at least 99.9% sequence identity) to the nucleotide sequence (e.g., genomic sequence, 16S sequence, CRISPR sequence) of the Prevotella histicola Strain B (NRRL accession number B 50329).

45. The method/solution/dried form/therapeutic composition/use of any one of claims 19 to 44, wherein the Prevotella histicola strain is the Prevotella histicola Strain B (NRRL accession number B 50329).

46. The method of any one of claim 1-18 or 32-44, wherein the subject is a human or a non-human mammal.
